SEQUENCE LISTING (63 sequences)

SEQ ID NO: 1 - protein, 806 aa, Artificial Sequence
  Synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIATVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 2 - protein, 831 aa, Artificial Sequence
  Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIATVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 3 - protein, 806 aa, Artificial Sequence
  Synthetic construct
QNITEEFYQSTCSAVSKGYLSALRTGWYTSVITIELSNIKENKCNGTDAKVKLIKQELDKYKNAVTELQLLMQSTPATNNRARAELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIATVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 4 - protein, 831 aa, Artificial Sequence
  Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARAELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIATVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 5 - protein, 806 aa, Artificial Sequence
  Synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRAFLGFLLGVGSAIASGVAVSKVLHLEGEVNKIKSALLSTNKAVVSLSNGVSVLTSKVLDLKNYIDKQLLPIVNKQSCSISNIATVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 6 - protein, 831 aa, Artificial Sequence
  Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRAFLGFLLGVGSAIASGVAVSKVLHLEGEVNKIKSALLSTNKAVVSLSNGVSVLTSKVLDLKNYIDKQLLPIVNKQSCSISNIATVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 7 - protein, 806 aa, Artificial Sequence
  Synthetic construct
QNITEEFYQSTCSAVSKGYLSALRTGWYTSVITIELSNIKENKCNGTDAKVKLIKQELDKYKNAVTELQLLMQSTPATNNRARAELPRFMNYTLNNAKKTNVTLSKKRKRAFLGFLLGVGSAIASGVAVSKVLHLEGEVNKIKSALLSTNKAVVSLSNGVSVLTSKVLDLKNYIDKQLLPIVNKQSCSISNIATVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 8 - protein, 831 aa, Artificial Sequence
  Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARAELPRFMNYTLNNAKKTNVTLSKKRKRAFLGFLLGVGSAIASGVAVSKVLHLEGEVNKIKSALLSTNKAVVSLSNGVSVLTSKVLDLKNYIDKQLLPIVNKQSCSISNIATVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 9 - protein, 806 aa, Artificial Sequence
  Synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLPGEVNKIKSALLSTNKAVVSLSNGVSVLTSKVLDLKNYIDKQLLPIVNKQSCSIPNIATVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 10 - protein, 831 aa, Artificial Sequence
  Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLPGEVNKIKSALLSTNKAVVSLSNGVSVLTSKVLDLKNYIDKQLLPIVNKQSCSIPNIATVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 11 - protein, 806 aa, Artificial Sequence
  Synthetic construct
QNITEEFYQSTCSAVSKGYLSALRTGWYTSVITIELSNIKENKCNGTDAKVKLIKQELDKYKNAVTELQLLMQSTPATNNRARAELPRFMNYTLNNAKKTNVTLSKKRKRAFLGFLLGVGSAIASGVAVSKVLHLPGEVNKIKSALLSTNKAVVSLSNGVSVLTSKVLDLKNYIDKQLLPIVNKQSCSIPNIATVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 12 - protein, 831 aa, Artificial Sequence
  Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARAELPRFMNYTLNNAKKTNVTLSKKRKRAFLGFLLGVGSAIASGVAVSKVLHLPGEVNKIKSALLSTNKAVVSLSNGVSVLTSKVLDLKNYIDKQLLPIVNKQSCSIPNIATVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 13 - protein, 741 aa, Artificial Sequence
  Synthetic construct
QNITEEFYQSTCSAVSKGYLSALRTGWYTSVITIELSNIKENKCNGTDAKVKLIKQELDKYKNAVTELQLLMQSTPATNNRARAELPRFMNYTLNNAKKTNVTLSKKRKRAFLGFLLGVGSAIASGVAVSKVLHLPGEVNKIKSALLSTNKAVVSLSNGVSVLTSKVLDLKNYIDKQLLPIVNKQSCSIPNIATVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKRSANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 14 - protein, 766 aa, Artificial Sequence
  Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARAELPRFMNYTLNNAKKTNVTLSKKRKRAFLGFLLGVGSAIASGVAVSKVLHLPGEVNKIKSALLSTNKAVVSLSNGVSVLTSKVLDLKNYIDKQLLPIVNKQSCSIPNIATVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKRSANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 15 - protein, 806 aa, Artificial Sequence
  Synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 16 - protein, 831 aa, Artificial Sequence
  Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 17 - protein, 495 aa, Artificial Sequence
  Synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIATVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGK

SEQ ID NO: 18 - protein, 520 aa, Artificial Sequence
  Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIATVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGK

SEQ ID NO: 19 - protein, 495 aa, Artificial Sequence
  Synthetic construct
QNITEEFYQSTCSAVSKGYLSALRTGWYTSVITIELSNIKENKCNGTDAKVKLIKQELDKYKNAVTELQLLMQSTPATNNRARAELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIATVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGK

SEQ ID NO: 20 - protein, 520 aa, Artificial Sequence
  Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARAELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIATVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGK

SEQ ID NO: 21 - protein, 806 aa, Artificial Sequence; identical to SEQ ID NO: 5
  Synthetic construct

SEQ ID NO: 22 - protein, 520 aa, Artificial Sequence
  Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRAFLGFLLGVGSAIASGVAVSKVLHLEGEVNKIKSALLSTNKAVVSLSNGVSVLTSKVLDLKNYIDKQLLPIVNKQSCSISNIATVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGK

SEQ ID NO: 23 - protein, 495 aa, Artificial Sequence
  Synthetic construct
QNITEEFYQSTCSAVSKGYLSALRTGWYTSVITIELSNIKENKCNGTDAKVKLIKQELDKYKNAVTELQLLMQSTPATNNRARAELPRFMNYTLNNAKKTNVTLSKKRKRAFLGFLLGVGSAIASGVAVSKVLHLEGEVNKIKSALLSTNKAVVSLSNGVSVLTSKVLDLKNYIDKQLLPIVNKQSCSISNIATVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGK

SEQ ID NO: 24 - protein, 520 aa, Artificial Sequence
  Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARAELPRFMNYTLNNAKKTNVTLSKKRKRAFLGFLLGVGSAIASGVAVSKVLHLEGEVNKIKSALLSTNKAVVSLSNGVSVLTSKVLDLKNYIDKQLLPIVNKQSCSISNIATVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGK

SEQ ID NO: 25 - protein, 495 aa, Artificial Sequence
  Synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLPGEVNKIKSALLSTNKAVVSLSNGVSVLTSKVLDLKNYIDKQLLPIVNKQSCSIPNIATVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGK

SEQ ID NO: 26 - protein, 520 aa, Artificial Sequence
  Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLPGEVNKIKSALLSTNKAVVSLSNGVSVLTSKVLDLKNYIDKQLLPIVNKQSCSIPNIATVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGK

SEQ ID NO: 27 - protein, 495 aa, Artificial Sequence
  Synthetic construct
QNITEEFYQSTCSAVSKGYLSALRTGWYTSVITIELSNIKENKCNGTDAKVKLIKQELDKYKNAVTELQLLMQSTPATNNRARAELPRFMNYTLNNAKKTNVTLSKKRKRAFLGFLLGVGSAIASGVAVSKVLHLPGEVNKIKSALLSTNKAVVSLSNGVSVLTSKVLDLKNYIDKQLLPIVNKQSCSIPNIATVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGK

SEQ ID NO: 28 - protein, 520 aa, Artificial Sequence
  Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARAELPRFMNYTLNNAKKTNVTLSKKRKRAFLGFLLGVGSAIASGVAVSKVLHLPGEVNKIKSALLSTNKAVVSLSNGVSVLTSKVLDLKNYIDKQLLPIVNKQSCSIPNIATVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGK

SEQ ID NO: 29 - protein, 495 aa, Artificial Sequence
  Synthetic construct
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGK

SEQ ID NO: 30 - protein, 520 aa, Artificial Sequence
  Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGK

SEQ ID NO: 31 - protein, 574 aa, Artificial Sequence
  Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 32 - protein, 574 aa, Artificial Sequence
  Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIATVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 33 - protein, 25 aa, Artificial Sequence
  Synthetic construct
MELLILKANAITTILTAVTFCFASG

SEQ ID NO: 34 - protein, 84 aa, Artificial Sequence
  Synthetic construct
QNITEEFYQSTCSAVSKGYLSALRTGWYTSVITIELSNIKENKCNGTDAKVKLIKQELDKYKNAVTELQLLMQSTPPTNNRARR

SEQ ID NO: 35 - protein, 84 aa, Artificial Sequence
  Synthetic construct
QNITEEFYQSTCSAVSKGYLSALRTGWYTSVITIELSNIKENKCNGTDAKVKLIKQELDKYKNAVTELQLLMQSTPATNNRARR

SEQ ID NO: 36 - protein, 4 aa, Artificial Sequence
  Synthetic construct
RARR

SEQ ID NO: 37 - protein, 4 aa, Artificial Sequence
  Synthetic construct
RARA

SEQ ID NO: 38 - protein, 27 aa, Artificial Sequence
  Synthetic construct
ELPRFMNYTLNNAKKTNVTLSKKRKRR

SEQ ID NO: 39 - protein, 6 aa, Artificial Sequence
  Synthetic construct
KKRKRR

SEQ ID NO: 40 - protein, 6 aa, Artificial Sequence
  Synthetic construct
KKRKRA

SEQ ID NO: 41 - protein, 19 aa, Artificial Sequence
  Synthetic construct
FLGFLLGVGSAIASGVAVS

SEQ ID NO: 42 - protein, 388 aa, Artificial Sequence
  Synthetic construct
FLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTN

SEQ ID NO: 43 - protein, 388 aa, Artificial Sequence
  Synthetic construct
FLGFLLGVGSAIASGVAVSKVLHLEGEVNKIKSALLSTNKAVVSLSNGVSVLTSKVLDLKNYIDKQLLPIVNKQSCSISNIATVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTN

SEQ ID NO: 44 - protein, 26 aa, Artificial Sequence
  Synthetic construct
IMITTIIIVIIVILLSLIAVGLLLYC

SEQ ID NO: 45 - protein, 24 aa, Artificial Sequence
  Synthetic construct
KARSTPVTLSKDQLSGINNIAFSN

SEQ ID NO: 46 - protein, 365 aa, Artificial Sequence
  Synthetic construct
KVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGK

SEQ ID NO: 47 - protein, 365 aa, Artificial Sequence
  Synthetic construct
KVLHLEGEVNKIKSALLSTNKAVVSLSNGVSVLTSKVLDLKNYIDKQLLPIVNKQSCSISNIATVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGK

SEQ ID NO: 48 - protein, 244 aa, Artificial Sequence
  Synthetic construct
ANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 49 - protein, 246 aa, Artificial Sequence
  Synthetic construct
RSANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 50 - protein, 309 aa, Artificial Sequence
  Synthetic construct
NGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 51 - protein, 309 aa, Artificial Sequence
  Synthetic construct
NGLPGPIGPPGPRGRTGDAGPVGPPGPPGPPGPPGPPSAGFDFSFLPQPPQEKAHDGGRYYRNDDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 52 - protein, 311 aa, Artificial Sequence
  Synthetic construct
RSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 53 - protein, 311 aa, Artificial Sequence
  Synthetic construct
GSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 54 - protein, 244 aa, Artificial Sequence
  Synthetic construct
ANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKSSRLPIIDVAPLDVGAPDQEFGFDVGPVCFL

SEQ ID NO: 55 - protein, 246 aa, Artificial Sequence
  Synthetic construct
RSANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKSSRLPIIDVAPLDVGAPDQEFGFDVGPVCFL

SEQ ID NO: 56 - protein, 309 aa, Artificial Sequence
  Synthetic construct
NGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKSSRLPIIDVAPLDVGAPDQEFGFDVGPVCFL

SEQ ID NO: 57 - protein, 309 aa, Artificial Sequence
  Synthetic construct
NGLPGPIGPPGPRGRTGDAGPVGPPGPPGPPGPPGPPSAGFDFSFLPQPPQEKAHDGGRYYRNDDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKSSRLPIIDVAPLDVGAPDQEFGFDVGPVCFL

SEQ ID NO: 58 - protein, 311 aa, Artificial Sequence
  Synthetic construct
RSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKSSRLPIIDVAPLDVGAPDQEFGFDVGPVCFL

SEQ ID NO: 59 - protein, 311 aa, Artificial Sequence
  Synthetic construct
GSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKSSRLPIIDVAPLDVGAPDQEFGFDVGPVCFL

SEQ ID NO: 60 - protein, 234 aa, Artificial Sequence
  Synthetic construct
DEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVQLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCF

SEQ ID NO: 61 - protein, 244 aa, Artificial Sequence
  Synthetic construct
EPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVQLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

SEQ ID NO: 62 - protein, 245 aa, Artificial Sequence
  Synthetic construct
SEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVQLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

SEQ ID NO: 63 - protein, 246 aa, Artificial Sequence
  Synthetic construct
RSEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVQLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

(19) United States
(12) Patent Application Publication
(10) Pub. No.: US 2023/0256076 A1
(43) Pub. Date: Aug. 17, 2023

(54) RSV VACCINE COMPOSITIONS, METHODS, AND USES THEREOF

(71) Applicant: Sichuan Clover Biopharmaceuticals, Inc., Chengdu Sichuan (CN)

(72) Inventors: Peng LIANG, Chengdu Sichuan (CN); Joshua LIANG, Chengdu Sichuan (CN)

(73) Assignee: Sichuan Clover Biopharmaceuticals, Inc., Chengdu Sichuan (CN)

(21) Appl. No.: 18/009,150

(22) PCT Filed: Jun. 10, 2021
(86) PCT No.: PCT/CN2021/099286

§ 371 Date: Dec. 8, 2022

Related U.S. Application Data
(63) Continuation of application No. PCT/CN2020/095295, filed on Jun. 10, 2020.
Application No. PCT/CN2020/095295 is a continuation of application No. PCT/CN2021/087045, filed on Apr. 13, 2021.

(30) Foreign Application Priority Data
Jun. 10, 2020 (WO) PCT/CN2020/095295
Apr. 13, 2021 (WO) PCT/CN2021/087045

(51) Int. Cl. A61K 39/12; A61K 9/00; A61P 31/14

(52) U.S. Cl. CPC A61K 39/12; A61K 9/0019; A61K 9/0043; A61P 31/14; A61K 2039/543

(57) ABSTRACT

Provided are immunogenic compositions including recombinant peptides and proteins comprising respiratory syncytial virus (RSV) viral antigens and immunogens, e.g., RSV F protein peptides. The immunogenic composition comprises a secreted fusion protein comprising a soluble RSV viral antigen joined by in-frame a disulfide bond-linked trimeric fusion protein. The immunogenic compositions are useful for generating an immune response, e.g., for treating or preventing an RSV infection. The immunogenic compositions may be used in a vaccine composition, e.g., as part of a prophylactic and/or therapeutic vaccine. Also provided herein are methods for producing the recombinant peptides and proteins, prophylactic, therapeutic, and/or diagnostic methods, and related kits.

### CROSS-REFERENCE TO RELATED APPLICATIONS

This application claims priority to and the benefit of International Patent Application Nos. PCT/CN2020/095295, filed Jun. 10, 2020, and PCT/CN2021/087045, filed Apr. 13, 2021, the disclosures of which applications are incorporated herein by reference in their entireties for all purposes.

### SUBMISSION OF SEQUENCE LISTING AS ASCII TEXT FILE

The content of the following submission on ASCII text file is incorporated herein by reference in its entirety: a computer readable form (CRF) of the Sequence Listing (file name: 165762000242SEQLIST.TXT, date recorded: Jun. 9, 2021, size: 229 KB).

### FIELD

The present disclosure relates in some aspects to immunogenic compositions including recombinant peptides and proteins comprising respiratory syncytial virus (RSV) viral antigens and immunogens, e.g., RSV F protein peptides, for treating and/or preventing an RSV infection.

### BACKGROUND

Respiratory syncytial virus (RSV) causes respiratory tract infections in adults and children, and is a leading cause of lower respiratory tract infections and hospitalization during infancy and childhood. Despite RSV's high infection rate, treatments, including prophylactic, therapeutic, and vaccine, are limited or unavailable. Improved approaches are needed for the treatment of RSV. Provided herein are compositions, methods, uses, and articles of manufacture that meet such and other needs.

### SUMMARY

In one aspect, provided herein is a protein comprising a plurality of recombinant polypeptides, each recombinant polypeptide comprising a respiratory syncytial virus (RSV) F protein peptide or a fragment or epitope thereof linked to a C-terminal propeptide of collagen, wherein the C-terminal propeptides of the recombinant polypeptides form inter-polypeptide disulfide bonds. In some embodiments, the RSV is of subtype A or subtype B. In some embodiments, the epitope is a linear epitope or a conformational epitope.

In some embodiments, disclosed herein are recombinant subunit vaccines that comprise an ecto-domain (e.g., without transmembrane and cytoplasmic domains) of an RSV F protein or its fragments which is fused in-frame to a C-propeptide of a collagen that is capable of forming disulfide bond-linked homo-trimer. The resulting recombinant subunit vaccines, such as an F-trimer, can be expressed and purified from transfected cells, and are expected to be in native-like conformation in trimeric form. This solves the problems of mis-folding of a viral antigen often encountered when it is expressed as a recombinant peptide or protein in soluble forms without the transmembrane and/or cytoplasmic domains. Such mis-folded viral antigens do not faithfully preserve the native viral antigen conformation, and often fail to evoke neutralizing antibodies.

In some embodiments, the F protein peptide comprises an F1 subunit peptide, an F2 subunit peptide, or any combination thereof, and the protein comprises three recombinant polypeptides. In some embodiments, the F protein peptide comprises a signal peptide, a heptad-repeat C (HRC) peptide, a pep27 peptide, a fusion peptide (FP), a heptad-repeat A (HRA) peptide, a Domain I peptide, a Domain U peptide, or heptad-repeat B (HRB) peptide, or any combination thereof. In some embodiments, the F protein peptide comprises an F1 subunit but not an F2 subunit of the F protein, or vice versa. In some embodiments, the F protein peptide comprises an F1 subunit and an F2 subunit of the F protein, optionally without pep27, and optionally wherein the F1 subunit and the F2 subunit are linked by a disulfide bond or an artificially introduced linker. In some embodiments, the F protein peptide does not comprise a transmembrane (TM) domain peptide and/or a cytoplasm (CP) domain peptide. In some embodiments, the F protein peptide comprises a protease cleavage site, wherein the protease is optionally furin, trypsin, factor Xa, thrombin, or cathepsin L. In some embodiments, the F protein peptide does not comprise a protease cleavage site, wherein the protease is optionally furin, trypsin, factor Xa, thrombin, or cathepsin L.

In some embodiments, the F protein peptide is soluble or does not directly bind to a lipid bilayer, e.g., a membrane or viral envelope. In some embodiments, F protein peptides are the same or different among the recombinant polypeptides of the protein. In some embodiments, the F protein peptide is directly fused to the C-terminal propeptide, or is linked to the C-terminal propeptide via a linker, such as a linker comprising glycine-X-Y repeats, wherein X and Y are independently any amino acid and optionally proline or hydroxyproline.

In some embodiments, the protein is soluble or does not directly bind to a lipid bilayer, e.g., a membrane or viral envelope. In some embodiments, the protein is capable of forming a rosette-like oligomer comprising F protein peptide trimers. In some embodiments, the protein is capable of binding to a cell surface attachment factor or receptor of a subject, optionally wherein the subject is a mammal such as a primate, e.g., human.

In some embodiments, the C-terminal propeptide is of human collagen. In some embodiments, the C-terminal propeptide comprises a C-terminal polypeptide of proα1(1), proα1(II), proα1(III), proα1(V), proα1(XI), proα2(I), proα2(V), proα2(XI), or proα3(XI), or a fragment thereof. In some embodiments, the C-terminal propeptides are the same or different among the recombinant polypeptides. In some embodiments, the C-terminal propeptide comprises any of SEQ ID NOs: 48-63 or an amino acid sequence at least 90% identical thereto capable of forming inter-polypeptide disulfide bonds and trimerizing the recombinant polypeptides.

In some embodiments, the F protein peptide in each recombinant polypeptide is in a prefusion conformation or a postfusion conformation, optionally wherein the protein comprises a rosette-like oligomer comprising F protein peptide trimers as crutch-shaped rods. In any of the preceding embodiments, the F protein peptide in each recombinant polypeptide can comprise any of SEQ ID NOs: 17-47 or an amino acid sequence at least 80% identical thereto.

In any of the preceding embodiments, the recombinant polypeptide can comprise any of SEQ ID NOs: 1-16 or an amino acid sequence at least 80% identical thereto. In any of the preceding embodiments, the recombinant polypeptide can comprise any of SEQ ID NOs: 17-47 or an amino acid sequence at least 80% identical thereto directly or indirectly linked to any of SEQ ID NOs: 48-63 or an amino acid sequence at least 90% identical thereto.

Also provided herein is an immunogen comprising a protein provided herein. Provided herein is a protein nanoparticle comprising protein provided herein directly or indirectly linked to a nanoparticle. Provided herein is a virus-like particle (VLP) comprising a protein provided herein.

Also provided herein is an isolated nucleic acid encoding one, two, three or more of the recombinant polypeptides of the protein provided herein. In some embodiments, a polypeptide encoding the F protein peptide is fused in-frame to a polypeptide encoding the C-terminal propeptide of collagen. In some embodiments, the isolated nucleic acid provided herein is operably linked to a promoter.

In some embodiments, the isolated nucleic acid provided herein is a DNA molecule. In some embodiments, the isolated nucleic acid provided herein is an RNA molecule, optionally an mRNA molecule such as a nucleoside-modified mRNA, a non-amplifying mRNA, a self-amplifying mRNA, or a trans-amplifying mRNA.

Also provided herein is a vector comprising an isolated nucleic acid provided herein. In some embodiments, the vector is a viral vector.

In some aspects, provided herein is a virus, a pseudovirus, or a cell comprising vector provided herein, optionally wherein the virus or cell has a recombinant genome. In some aspects, provided herein is an immunogenic composition comprising the protein, immunogen, protein nanoparticle, VLP, isolated nucleic acid, vector, virus, pseudovirus, or cell provided herein, and a pharmaceutically acceptable carrier.

Also provided herein is a vaccine comprising an immunogenic composition provided herein and optionally an adjuvant, wherein the vaccine is optionally a subunit vaccine. In some embodiments, the vaccine is a prophylactic and/or therapeutic vaccine.

In some aspects, provided herein is a method of producing a protein, comprising: expressing the isolated nucleic acid or vector provided herein in a host cell to produce the protein as provided herein; and purifying the protein. Provided herein is a protein produced by a method provided herein.

Provided herein are methods for generating an immune response to an F protein peptide or fragment or epitope thereof of an RSV in a subject, comprising administering to the subject an effective amount of the protein, immunogen, protein nanoparticle, VLP, isolated nucleic acid, vector, virus, pseudovirus, cell, immunogenic composition, or vaccine as provided herein to generate the immune response. In some embodiments, the method provided herein is for treating or preventing infection with the RSV. In some embodiments, generating the immune response inhibits or reduces replication of the RSV in the subject. In some embodiments, the immune response comprises a cell-mediated response and/or a humoral response, optionally comprising production of one or more neutralizing antibody, such as a polyclonal antibody or a monoclonal antibody. In some embodiments, the immune response is against the F protein peptide or fragment or epitope thereof of the RSV but not against the C-terminal propeptide. In some embodiments, the administering to the subject does not lead to antibody dependent enhancement (ADE) in the subject due to prior exposure to one or more RSV. In some embodiments, the administering does not lead to antibody dependent enhancement (ADE) in the subject when subsequently exposed to one or more RSV. In some embodiments, the method further comprises a priming step and/or a boosting step. In some embodiments, the administering step is performed via topical, transdermal, subcutaneous, intradermal, oral, intranasal (e.g., intranasal spray), intratracheal, sublingual, buccal, rectal, vaginal, inhaled, intravenous (e.g., intravenous injection), intraarterial, intramuscular (e.g., intramuscular injection), intracardiac, intraosseous, intraperitoneal, transmucosal, intravitreal, subretinal, intraarticular, peri-articular, local, or epicutaneous administration. In some embodiments, the effective amount is administered in a single dose or a series of doses separated by one or more interval. In some embodiments, the effective amount is administered without an adjuvant. In some embodiments, the effective amount is administered with an adjuvant.

Provided herein are methods comprising administering to a subject an effective amount of a protein provided herein to generate in the subject a neutralizing antibody or neutralizing antisera to the RSV. In some embodiments, the subject is a mammal, optionally a human or a non-human primate. In some embodiments, the method further comprises isolating the neutralizing antibody or neutralizing antisera from the subject. In some embodiments, the method further comprises administering an effective amount of the isolated neutralizing antibody or neutralizing antisera to a human subject via passive immunization to prevent or treat an infection by the RSV. In some embodiments, the neutralizing antibody or neutralizing antisera to the RSV comprises polyclonal antibodies to the RSV F protein peptide or fragment or epitope thereof, optionally wherein the neutralizing antibody or neutralizing antisera is free or substantially free of antibodies to the C-terminal propeptide of collagen. In some embodiments, the neutralizing antibody comprises a monoclonal antibody to the RSV F protein peptide or fragment or epitope thereof, optionally wherein the neutralizing antibody is free or substantially free of antibodies to the C-terminal propeptide of collagen.

In some aspects, the protein, immunogen, protein nanoparticle, VLP, isolated nucleic acid, vector, virus, pseudovirus, cell, immunogenic composition, or vaccine provided herein, is for use in inducing an immune response to an RSV in a subject, and/or in treating or preventing an infection by the RSV.

In some aspects, provided herein is use of the protein, immunogen, protein nanoparticle, VLP, isolated nucleic acid, vector, virus, pseudovirus, cell, immunogenic composition, or vaccine provided herein, for inducing an immune response to an RSV in a subject, and/or for treating or preventing an infection by the RSV. In some aspects, provided herein is use of the protein, immunogen, protein nanoparticle, VLP, isolated nucleic acid, vector, virus, pseudovirus, cell, immunogenic composition, or vaccine provided herein, for the manufacture of a medicament or a prophylactic for inducing an immune response to an RSV in a subject, and/or for treating or preventing an infection by the RSV.

Also provided herein are methods for analyzing a sample, comprising: contacting a sample with the protein provided herein, and detecting a binding between the protein and an analyte capable of specific binding to the F protein peptide or fragment or epitope thereof of the RSV. In some embodiments, the analyte is an antibody, a receptor, or a cell recognizing the F protein peptide or fragment or epitope thereof. In some embodiments, the binding indicates the presence of the analyte in the sample, and/or an infection by the RSV in a subject from which the sample is derived.

Provided herein are kits comprising the protein provided herein and a substrate, pad, or vial containing or immobilizing the protein, optionally wherein the kit is an ELISA or lateral flow assay kit.

### BRIEF DESCRIPTION OF THE DRAWINGS

FIGS. 1A-1C show expression levels and purification of exemplary fusion peptides comprising an RSV F protein peptide. FIG. 1A shows a schematic representation of an exemplary fusion peptide comprising an extracellular F domain fused to a trimerization peptide. FIG. 1B shows 8% SDS-PAGE analysis of exemplary fusion peptide expression from a serum-free fed-batch cell culture. 10 μL of cell-free conditioned medium from day 1 to day 12 were separated under non-reducing condition, followed by Coomassie blue staining. FIG. 1C shows a purity evaluation of an exemplary fusion peptide by SEC-HPLC, the main peak area of the exemplary protein was 94.6% with OD₂₈₀ detection.

FIGS. 2A-2C show the characterization of an exemplary purified fusion peptide comprising an RSV F protein peptide. FIG. 2A shows SDS-PAGE and western blot analysis of an exemplary fusion peptide comprising an RSV F protein peptide under non-reducing and reducing conditions. 2 μg of purified protein was loaded for Coomassie blue staining by 8% SDS-PAGE, and 0.1 μg of purified protein was loaded for western blot by using antibodies specific to F and the propeptide of collagen respective. FIG. 2B shows negative staining electron micrograph of exemplary fusion peptide proteins comprising an RSV F protein peptide, which shows crutch-shaped molecules in the form of individuals or mostly rosette-like oligomers. Examples of individual and rosette-like molecules are shown below accompanied with diagrams of their structures. FIG. 2C shows binding studies by biolayer interferometry of palivizumab and an exemplary fusion peptide comprising an RSV F protein peptide. 5 μg/mL palivizumab was first immobilized on protein A sensors, and sensors were then dipped in varying concentrations of exemplary fusion peptide to measure binding kinetics. Fitting the resulting curves to a 1:1 binding model by subtracting buffer reference resulted in Kₒₙ and K_{dis}, and values are indicated in the table below. The resulting K_{D} for palivizumab and the exemplary fusion peptide comprising an RSV F protein peptide is below one picomolar.

FIGS. 3A-3E show immunization with an exemplary fusion peptide comprising an RSV F protein peptide protects against RSV infection. FIG. 3A shows a schematic outline of the experimental approach: mice were immunized at day 0 and 21 and followed by intranasal (i.n.) challenge with RSV at day 49 after sera collection. FIG. 3B shows serum anti-F IgG ELISA titers against the purified exemplary fusion peptides comprising RSV F protein peptide. FIG. 3C is a viral microneutralization assay showing titers of serum neutralizing antibody that provide 50% inhibition of CPE formation against RSV infection. FIG. 3D shows titers of RSV 5 days post-challenge by plaque assay in the lungs of immunized mice. Values represent plaques per gram of lung tissue. FIG. 3E shows palivizumab competitive IgG titers determined by dilutions of serum samples that provide 50% inhibition of pilivizumab binding to heat-inactivated RSV (HI-RSV) particles. Values represent as log 2 and means±SEM.

FIG. 4 shows immunization with an exemplary fusion peptide comprising an RSV F protein peptide protects from vaccine-induced disease enhancement. Lung tissues collected 5 days after challenge were fixed in 10% neutral buffered formalin, paraffin-embedded, sectioned at 5 μm and stained with H&E, pictures were captured at 200× magnification.

### DETAILED DESCRIPTION

In some embodiments, compositions and methods of use of recombinant soluble surface antigens from RNA viruses in covalently linked trimeric forms are disclosed. In some embodiments, the resulting fusion proteins are secreted as disulfide bond-linked homo-trimers, which are more stable in structure, while preserving the conformations of native-like trimeric viral antigens, thereby can be used as more effective vaccines against these dangerous pathogens.

In some embodiments, disclosed herein are methods for using viral antigen trimers as a vaccine or as part of a multivalent vaccine to prevent viral infections, without or with adjuvant, or with more than one adjuvant, optionally via either intra-muscular injections or intra-nasal administrations.

In some embodiments, disclosed herein are methods for using viral antigen trimers as an antigen for diagnosis of viral infections through detection of antibodies, e.g., IgM or IgG, that recognize the viral antigen, such as neutralizing antibodies.

In some embodiments, disclosed herein are methods for using viral antigen trimers as an antigen to generate polyclonal or monoclonal antibodies which can be used for passive immunization, e.g., neutralizing mAb for treating RSV infection in infants.

In some embodiments, disclosed herein is a viral antigen trimer as a vaccine or as part of a multivalent vaccine, wherein the vaccine comprises a plurality of trimeric subunit vaccines comprising viral antigens of the same protein of a virus or comprising viral antigens of two or more different proteins of one or more viruses or one or more strains of the same virus.

In some embodiments, disclosed herein is a monovalent vaccine comprising a viral antigen trimer disclosed herein. In some embodiments, disclosed herein is a bi-valent vaccine comprising a viral antigen trimer disclosed herein. In some embodiments, disclosed herein is a tri-valent vaccine comprising a viral antigen trimer disclosed herein. In some embodiments, disclosed herein is a quadrivalent vaccine comprising a viral antigen trimer disclosed herein.

In some embodiments, disclosed herein is a monovalent vaccine comprising an F-Trimer disclosed herein. In some embodiments, disclosed herein is a bi-valent vaccine comprising an F-Trimer disclosed herein. In some embodiments, disclosed herein is a bi-valent vaccine comprising at least one F-Trimer comprising a first F protein antigen and at least one F-Trimer comprising a second F protein antigen. In some embodiments, the first and second F protein antigens are from the same F protein of one or more virus species or strains/subtypes, or from two or more different F proteins of one or more virus species or one or more strains/subtypes of the same virus species. In some embodiments, disclosed herein is a tri-valent vaccine comprising an F-Trimer disclosed herein. In some embodiments, disclosed herein is a tri-valent vaccine comprising at least one F-Trimer comprising a first F protein antigen, at least one F-Trimer comprising a second F protein antigen, and at least one F-Trimer comprising a third F protein antigen. In some embodiments, the first, second and third F protein antigens are from the same F protein of one or more virus species or strains/subtypes, or from two, three, or more different F proteins of one or more virus species or one or more strains/subtypes of the same virus species. In some embodiments, disclosed herein is a quadrivalent vaccine comprising an F-Trimer disclosed herein. In some embodiments, disclosed herein is quadrivalent vaccine comprising at least one F-Trimer comprising a first F protein antigen, at least one F-Trimer comprising a second F protein antigen, at least one F-Trimer comprising a third F protein antigen, and at least one F-Trimer comprising a fourth F protein antigen. In some embodiments, the first, second, third, and fourth F protein antigens are from the same F protein of one or more virus species or strains/subtypes, or from two, three, four, or more different F proteins of one or more virus species or one or more strains/subtypes of the same virus species.

Provided herein are immunogenic compositions, methods, and uses of fusion peptides and proteins comprising RSV viral antigens or immunogens for the treatment, e.g., prophylactic, therapeutic, of RSV infection. Respiratory syncytial virus (RSV) is considered to be the main cause of acute lower respiratory tract infection (ALRTI) among infants and young children, and accounts for over 7,000 to 20,000 child deaths each year worldwide. RSV infection is the second most common cause of infant mortality in the developing world. In addition, RSV can lead to serious diseases in the elderly and immunocompromised populations. Despite the disease burden caused by RSV, there is currently no approved vaccine. Palivizumab (SYNAGIS®), a potent prophylactic humanized mAb, is only available as a passive immunization measure for those infants at high-risk of contracting RSV.

Despite decades of research, RSV vaccine development has been unsuccessful for numerous reasons. For example, issues with production, stability, efficacy of RSV vaccine candidates have been difficult to overcome, and safety in particular has been a main concern since it was recognized that formalin-inactivated RSV (FI-RSV) vaccines mediated vaccine-induced disease enhancement (VED).

The proteins, including recombinant polypeptides and fusion proteins, comprising RSV viral antigens and immunogens provided herein are useful for effectively and safely treating (e.g., therapeutically, prophylactically) RSV infection. For example, the proteins comprising RSV viral antigens and immunogens provided herein treat RSV infection without meditated VED and/or antibody dependent enhancement (ADE). In addition, the proteins comprising RSV viral antigens and immunogens provided herein are easily produced, and demonstrate stability under high stress conditions such as, e.g., high temperature, extreme pH, and high and low osmolality. Thus, the proteins and immunogenic compositions provided herein circumvent and satisfy the issues of production, stability, safety, and efficacy that have hindered RSV vaccine development.

In some aspects, the RSV viral antigens and immunogens provided herein include the RSV glycoprotein (F), also referred to herein as an RSV F protein peptide or peptide. RSV F protein peptide is a homotrimeric type I transmembrane protein that mediates membrane and viral penetration into host cells. The RSV F protein peptide is synthesized as an F0 proprotein precursor and converted into a disulfide-linked F1 and F2 mature form after cleavage by furin at two sites. The RSV F protein peptide is highly conserved between RSV A and B strains. Neutralizing antibodies, such as palivizumab, target antigenic sites of F and provide protection against respiratory disease caused by RSV infection.

In some embodiments, the protein comprising the RSV viral antigen or immunogen, e.g., RSV F protein peptide, is capable of generating an immune response, e.g., an immune response to the RSV F peptide protein. In some embodiments, the immune response inhibits or reduces replication of RSV in a subject, e.g., a patient. In some embodiments, the immune response includes production of one or more neutralizing antibodies, such as polyclonal and/or monoclonal antibodies. In some embodiments, the neutralizing antibodies inhibit or reduce replication of RSV in a subject, e.g., a patient. In some embodiments, administration of the protein, for example as an immunogenic composition, to the subject does not lead to antibody dependent enhancement (ADE) in the subject due to prior exposure to RSV. In some aspects, the protein comprising an RSV viral antigen and immunogen, e.g., RSV F protein peptide, is used as a vaccine.

In some embodiments, the RSV viral antigen and immunogen, e.g., RSV F protein peptide, is linked to a protein or peptide to form a fusion protein or recombinant polypeptide. In some embodiments, the protein or peptide to which the RSV viral antigen or immunogen is linked is capable of associating, e.g., covalently or non-covalently linking, with proteins or peptides, such as proteins or peptides of fusion proteins or recombinant polypeptides. Thus, in some cases, the protein or peptide to which the RSV viral antigen or immunogen is linked is a multimerization domain.

In some embodiments, the RSV viral antigen and immunogen, e.g., RSV F protein peptide, is linked to a propeptide of collagen, e.g., at the C-terminal of propeptide of collagen, to form a fusion peptide or recombinant polypeptide. Thus, in some embodiments, the protein provided herein comprises recombinant polypeptides containing RSV viral antigens and immunogens, e.g., RSV F protein peptides or a fragment or epitope thereof, linked to a C-terminal propeptide of collagen. In some embodiments, the propeptide of collagen is derived from the human C-propeptide of al collagen and is capable self-trimerization.

In some embodiments, linking the RSV viral antigen and immunogen, e.g., RSV F protein peptide, to a propeptide of collagen, e.g., at the C-terminal of propeptide of collagen, aids in the ability of the protein to generate an immune response. For example, the creation of the recombinant protein may preserve the tertiary and quaternary structures of the RSV F protein peptide, which may be important for the stability of the native conformation of the RSV F protein peptide, and in turn the availability of antigenic sites on the surface of the protein capable of eliciting an immune response, e.g., neutralizing antibodies. Additionally, linking of the RSV F protein peptide to a protein or peptide capable of self-trimerization allows the aggregation of the recombinant proteins, thus mimicking the native homotrimeric structure of the RSV F protein peptides on the viral envelope.

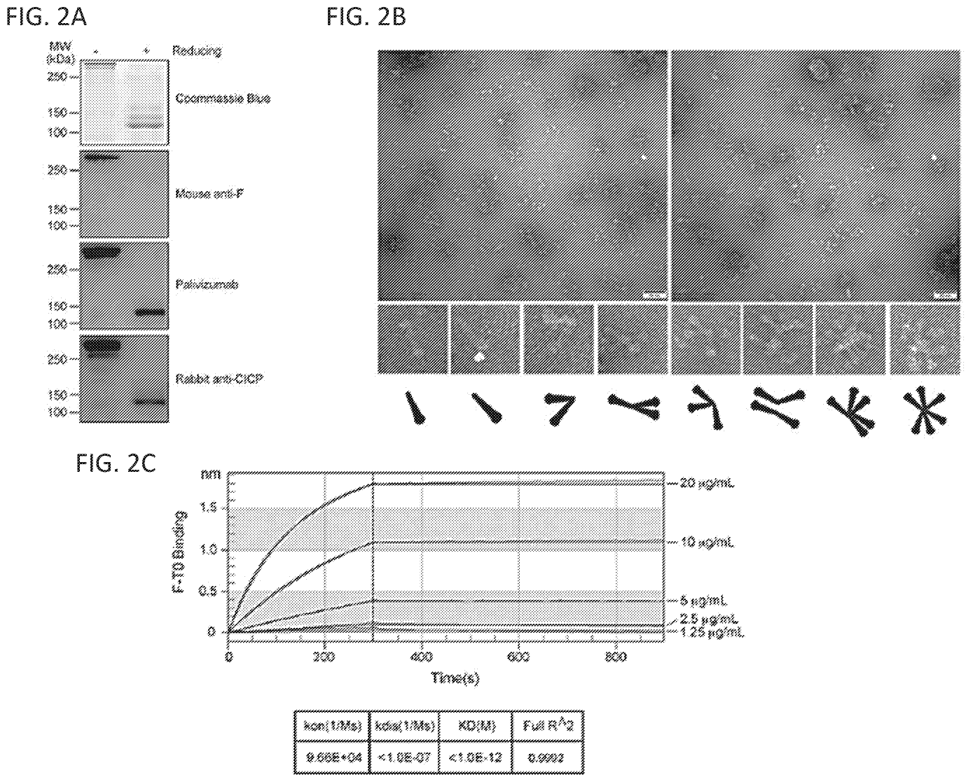

In some embodiments, linking the RSV F protein peptide to a C-terminal propeptide of collagen results in self-trimerized recombinant polypeptides. In some embodiments, the protein provided herein comprises a plurality of self-trimerized RSV F protein peptide and propeptide of collagen recombinant polypeptides, optionally where the plurality of recombinant proteins forms structures, e.g., rosettes (See, for example, FIG. 2B). In some embodiments, the trimeric nature of the recombinant proteins aids in the stability of the protein. In some embodiments, the macrostructure, e.g., rosettes, of a plurality of self-trimerized recombinant proteins aids in the stability of the protein. In some embodiments, the trimeric nature of the recombinant proteins and macrostructure, e.g., rosettes, of a plurality of self-trimerized recombinant proteins aids in the stability of the protein. In some embodiments, the trimeric nature of the recombinant proteins aids in the ability of the protein to generate an immune response. In some embodiments, the macrostructure, e.g., rosettes, of a plurality of self-trimerized recombinant proteins aids in the ability of the protein to generate an immune response. In some embodiments, the trimeric nature of the recombinant proteins and macrostructure of a plurality of self-trimerized recombinant proteins aids in the ability of the protein to generate an immune response.

Also provided herein are immunogenic compositions comprising the proteins provided herein, methods of producing proteins provided herein, methods of treating subjects with proteins and compositions provided herein, and kits.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Viral Antigens and Immunogens

Respiratory syncytial virus (RSV) is the most common cause of acute lower respiratory infection in infants and young children, and a major disease burden in the elderly. Despite the fact that the RSV virus was characterized half a century ago, there is currently no vaccine for RSV and development has been hampered by vaccine-mediated disease enhancement in children administered a formalin inactivated RSV in the 1960s. Challenges in antigen production, purity, stability, and potency of RSV vaccine candidates have also been impediments to development.

In some embodiments, the proteins provided herein comprise RSV viral antigens and/or immunogens. In some embodiments, the RSV viral antigens and/or immunogens herein are capable of promoting or stimulating a cell-mediated response and/or a humoral response. In some embodiments, the response, e.g., cell-mediated or humoral response, comprises the production of antibodies, e.g., neutralizing antibodies. In some embodiments, the neutralizing antibodies (NAbs) against viral antigens and/or immunogens provide adaptive immune defense against RSV exposure by blocking the infection of susceptible cells. In some embodiments, the efficacy of vaccines against several viruses is attributed to and/or correlated with their ability to elicit NAbs. In some embodiments, the RSV viral antigen or immunogen is an RSV F protein peptide disclosed herein.

The RSV F protein peptide is an envelope glycoprotein of respiratory syncytial viruses (RSVs). The RSV F protein peptide is translated as a single precursor polypeptide (designated F0). The RSV F protein mediates viral entry into cells and cell to cell fusion, is a target of neutralizing antibodies, and highly conserved between RSV A and B strains. F0 can be cleaved at Arg109 and Arg136 by cellular furin to three fragments, a shorter F2 polypeptide at the N-terminus covalently linked by two disulfides to a longer F1 polypeptide with an 18 amino acid fusion domain at the N-terminus and a hydrophobic membrane spanning region near the C-terminus; the intervening 27 amino acid fragment is released. Neutralizing monoclonal antibodies palivizumab and motavizumab bind to RSV F antigenic site II (Asn258-Val278) and have been shown to protect against both lower and upper respiratory RSV disease in high risk and term infants. The structures of the RSV F epitope polypeptides that bind these neutralizing antibodies are larger than the linear peptide and palivizumab binds with nanomolar and motavizumab picomolar affinity to RSV F. Modeling predicts that the full extent of the binding of palivizumab and motavizumab requires amino acids from one or two RSV F protomers, respectively. Therefore, preserving RSV F tertiary and quaternary structures may be important in the development of an RSV F vaccine to preserve the native conformation of this important neutralizing region.

In some embodiments, the F0 precursor polypeptide is 574 amino acids in length as set forth in SEQ ID NO: 31.

```
                10 20 30 40 50 60
MELLILKANAITTILTAVTFCFASGQNITEEFYQSTCSAVSKGYLSALRTGWYTSVITIE
                70 80 90 100 110 120
LSNIKENKCNGTDAKVKLIKQELDKYKNAVTELQLLMQSTPPTNNRARRFLPRFMNYTLN
              130 140 150 160 170 180
NAKKTNVTLSKKRKRRFLGFLLGVGSAIASGVAVSKVLHLEGEVNKIKSALLSTNKAVVS
              190 200 210 220 230 240
LSNGVSVLTSKVLDLKNYIDKQLLPIVNKQSCSISNIETVIEFQQKNNRLLEITREFSVN
              250 260 270 280 290 300
AGVTTPVSTYMLTNSELLSLINDMPITNDQKKLMSNNVQIVRQQSYSIMSIIKEEVLAYV
              310 320 330 340 350 360
VQLPLYGVIDTPCWKLHTSPLCTTNTKEGSNICLTRTDRGWYCDNAGSVSFFPQAETCKV
              370 380 390 400 410 420
QSNRVFCDTMNSLTLPSEINLCNVDIFNPKYDCKIMTSKTDVSSSVITSLGAIVSCYGKT
              430 440 450 460 470 480
KCTASNKNRGIIKTFSNGCDYVSNKGMDTVSVGNTLYYVNKQEGKSLYVKGEPIINFYDP
              490 500 510 520 530 540
LVFPSDEFDASISQVNEKINQSLAFIRKSDELLHNVNAGKSTTNIMITTIIIVIIVILLS
              550 560 570
LIAVGLLLYCKARSTPVTLSKDQLSGINNIAFSN
```

In some embodiments, the F0 precursor polypeptide is 574 amino acids in length as set forth in SEQ ID NO: 32.

```
                10 20 30 40 50 60
MELLILKANAITTILTAVTFCFASGQNITEEFYQSTCSAVSKGYLSALRTGWYTSVITIE
                70 80 90 100 110 120
LSNIKENKCNGTDAKVKLIKQELDKYKNAVTELQLLMQSTPATNNRARRELPRFMNYTLN
              130 140 150 160 170 180
NAKKTNVTLSKKRKRRFLGFLLGVGSAIASGVAVSKVLHLEGEVNKIKSALLSTNKAVVS
              190 200 210 220 230 240
LSNGVSVLTSKVLDLKNYIDKQLLPIVNKQSCSISNIATVIEFQQKNNRLLEITREFSVN
              250 260 270 280 290 300
AGVTTPVSTYMLTNSELLSLINDMPITNDQKKLMSNNVQIVRQQSYSIMSIIKEEVLAYV
              310 320 330 340 350 360
VQLPLYGVIDTPCWKLHTSPLCTTNTKEGSNICLTRTDRGWYCDNAGSVSFFPQAETCKV
              370 380 390 400 410 420
QSNRVFCDTMNSLTLPSEVNLCNVDIFNPKYDCKIMTSKTDVSSSVITSLGAIVSCYGKT
              430 440 450 460 470 480
KCTASNKNRGIIKTFSNGCDYVSNKGVDTVSVGNTLYYVNKQEGKSLYVKGEPIINFYDP
              490 500 510 520 530 540
LVFPSDEFDASISQVNEKINQSLAFIRKSDELLHNVNAGKSTTNIMITTIIIVIIVILLS
              550 560 570
LIAVGLLLYCKARSTPVTLSKDQLSGINNIAFSN
```

In some embodiments, the RSV F protein peptide herein comprises proline or alanine at residue 102. In some embodiments, the RSV F protein peptide herein comprises a substitution, deletion, and/or insertion at and/or around residue 102 in SEQ ID NO: 31 or 32. In some embodiments, the RSV F protein peptide herein comprises glutamic acid or alanine at residue 218. In some embodiments, the RSV F protein peptide herein comprises a substitution, deletion, and/or insertion at and/or around residue 218 in SEQ ID NO: 31 or 32. In some embodiments, the RSV F protein peptide herein comprises valine or isoleucine at residue 379. In some embodiments, the RSV F protein peptide herein comprises a substitution, deletion, and/or insertion at and/or around residue 379 in SEQ ID NO: 31 or 32. In some embodiments, the RSV F protein peptide herein comprises valine or methionine at residue 447. In some embodiments, the RSV F protein peptide herein comprises a substitution, deletion, and/or insertion at and/or around residue 447 in SEQ ID NO: 31 or 32. In some embodiments, the RSV F protein peptide herein comprises a substitution, deletion, and/or insertion at and/or around any one or more of proline or alanine at residue 102, glutamic acid or alanine at residue 218, valine or isoleucine at residue 379, and valine or methionine at residue 447. In some embodiments, the RSV F protein peptide herein comprises a substitution, deletion, and/or insertion at and/or around any one or more of residues 102, 218, 379, and 447 in SEQ ID NO: 31 or 32. In some embodiments, the RSV F protein peptide herein comprises a substitution, deletion, and/or insertion at and/or around any one or more of the other residues in SEQ ID NO: 31 or 32.

In some embodiments, the RSV F protein peptide herein comprises a substitution, deletion, and/or insertion at and/or around residue 106, 107, 108, and/or 109 in SEQ ID NO: 31 or 32. In some embodiments, the RSV F protein peptide herein comprises glutamine or asparagine at residue 108 and/or 109. In some embodiments, the RSV F protein peptide herein comprises glutamine at residues 108 and 109. In some embodiments, the RSV F protein peptide herein comprises asparagine at residues 108 and 109. In some embodiments, the RSV F protein peptide herein comprises a substitution, deletion, and/or insertion at and/or around residue 131, 132, 133, 134, 135, and/or 136 in SEQ ID NO: 31 or 32. In some embodiments, the RSV F protein peptide herein comprises glycine, arginine, glutamine or asparagine at and/or around residue 131, 132, 133, 134, 135, and/or 136 in SEQ ID NO: 31 or 32. In some embodiments, the RSV F protein peptide herein comprises glutamine at residues 131, 132, 133, 134, 135, and/or 136 in SEQ ID NO: 31 or 32. In some embodiments, the RSV F protein peptide herein comprises glutamine at residues 133, 135, and 136 in SEQ ID NO: 31 or 32.

In some embodiments, the RSV F protein peptide herein comprises a substitution, deletion, and/or insertion at and/or around residue 109, 136, 161, and/or 215 in SEQ ID NO: 31 or 32. In some embodiments, the RSV F protein peptide herein comprises alanine or proline at any one or more of residues 109, 136, 161, and/or 215 in SEQ ID NO: 31 or 32. In some embodiments, the RSV F protein peptide herein comprises alanine at residues 109 in SEQ ID NO: 31 or 32. In some embodiments, the RSV F protein peptide herein comprises alanine at residue 136 in SEQ ID NO: 31 or 32. In some embodiments, the RSV F protein peptide herein comprises alanine at residues 109 and 136 in SEQ ID NO: 31 or 32. In some embodiments, the RSV F protein peptide herein comprises proline at residue 161 in SEQ ID NO: 31 or 32. In some embodiments, the RSV F protein peptide herein comprises proline at residue 215 in SEQ ID NO: 31 or 32. In some embodiments, the RSV F protein peptide herein comprises proline at residues 161 and 215 in SEQ ID NO: 31 or 32. In some embodiments, the RSV F protein peptide herein comprises alanine at residues 109 and 136, and proline at residues 161 and 215 in SEQ ID NO: 31 or 32.

In some embodiments, the RSV F protein peptide herein comprises a substitution, deletion, and/or insertion at and/or around any one or more of residues 131-154 in SEQ ID NO: 31 or 32. In some embodiments, the RSV F protein peptide herein comprises a deletion of two, three, four, five, six, seven, eight, nine, ten, 11, 12, 13, 14, 15, 16, 17, 18 or more of residues 131-154 of SEQ ID NO: 31 or 32. In some embodiments, the RSV F protein peptide herein comprises a deletion of any one or more of residues 137-154 in SEQ ID NO: 31 or 32. In some embodiments, the RSV F protein peptide herein comprises a deletion of any one or more of residues 137-146 in SEQ ID NO: 31 or 32. In some embodiments, the RSV F protein peptide herein comprises glutamine at residues 133, 135, and 136 and a deletion of residues 137-146 in SEQ ID NO: 31 or 32.

In some embodiments, the RSV F protein peptide herein comprises amino acids 1-25 of the F0 precursor which is a signal peptide MELLILKANAITTILTAVTFCFASG (SEQ ID NO: 33). In some embodiments, the precursor polypeptide F0 forms a precursor trimer. In some embodiments, the RSV F protein peptide herein is proteolytically cleaved by one or more cellular proteases, e.g., at conserved furin consensus cleavage sites, to yield a Pep 27 polypeptide (also referred to as p27), an F1 polypeptide and an F2 polypeptide. In some embodiments, the Pep 27 polypeptide (e.g., amino acids 110-136 of the F0 precursor) is excised and in some aspects, does not form part of a mature RSV F trimer. In some embodiments, the F2 polypeptide (which may alternatively be referred to herein as “F2” or the “F2 subunit peptide”) comprises amino acid residues 26-109 of the F0 precursor. In some embodiments, the F1 polypeptide (which may alternatively be referred to herein as “F1” or the “F1 subunit peptide”) comprises amino acid residues 137-574 of the F0 precursor, and may comprise an extracellular region (e.g., residues 137-524), a transmembrane domain (e.g., residues 525-550), and a cytoplasmic domain (e.g., residues 551-574).

In some embodiments, the RSV F protein peptide herein comprises F1 and F2 polypeptides linked by disulfide-bonds to form a heterodimer which is referred to as an RSV F “protomer.” In some embodiments, the RSV F protein peptide herein comprises three protomers that form a RSV F trimer—which is thus a homotrimer of the three protomers. In some embodiments, the RSV F protein peptide herein is a mature RSV F trimer. In some embodiments, the RSV F protein peptide herein is membrane-bound. In some embodiments, the RSV F protein peptide herein is not membrane-bound. In some embodiments, the RSV F protein peptide herein is soluble and lacks the transmembrane and cytoplasmic regions or a fragment thereof. For example, conversion to a soluble form can be accomplished by truncating the RSV F protein at amino acid 513 (by removing amino acids 514 onwards), 514, 515, 516, 517, 518, 519, 520, 521, 522, 523, or 524. In nature the mature RSV F trimer mediates fusion of viral and cellular membranes. The profusion conformation of the mature RSV F trimer (which may be referred to herein as “pre-F” or prefusion) is highly unstable (metastable). However, once the RSV virus docks with the cell membrane, the RSV F protein trimer undergoes a series of conformational changes and transitions to a highly stable postfusion (“post-F”) conformation.

In some embodiments, the RSV viral antigen or immunogen comprises a signal peptide (SP) (e.g., amino acids 1-22 of SEQ ID NO: 31 or 32) or fragment and/or mutant sequence thereof, heptad-repeat C (HRC) (e.g., F2, which may be amino acids 23-109 of SEQ ID NO: 31 or 32) or fragment and/or mutant sequence thereof, furin cleavage site (FCS) (e.g., at the junction between amino acids 109/110 of SEQ ID NO: 31 or 32) or fragment and/or mutant sequence thereof, 27-mer fragment (pep27) (e.g., amino acids 110-136 of SEQ ID NO: 31 or 32) or fragment and/or mutant sequence thereof, putative fusion peptide (FP) (e.g., amino acids 137-155 of SEQ ID NO: 31 or 32) or fragment and/or mutant sequence thereof, heptad-repeat A (HRA) (e.g., amino acids 156-214 of SEQ ID NO: 31 or 32) or fragment and/or mutant sequence thereof, domains I and II (e.g., amino acids 215-476 of SEQ ID NO: 31 or 32) or fragment and/or mutant sequence thereof, heptad-repeat B (HRB) (e.g., amino acids 477-524 of SEQ ID NO: 31 or 32) or fragment and/or mutant sequence thereof, transmembrane (TM) (e.g., amino acids 525-550 of SEQ ID NO: 31 or 32) or fragment and/or mutant sequence thereof, and/or cytoplasm (CP) domain (e.g., amino acids 551-574 of SEQ ID NO: 31 or 32) or fragment and/or mutant sequence thereof, in any suitable combination.

In some embodiments, the RSV viral antigen or immunogen is an RSV F protein peptide of an RSV of subtype A. In some embodiments, the RSV viral antigen or immunogen is an RSV F protein peptide of an RSV of subtype A2. In some embodiments, the RSV viral antigen or immunogen is an RSV F protein peptide of an RSV of subtype B. In some cases, the RSV F protein peptide is conserved across RSV subtypes.

In some cases, the RSV viral antigen or immunogen is a fragment of an RSV F protein peptide. In some embodiments, the RSV viral antigen or immunogen is an epitope of RSV F protein peptide. In some embodiments, the epitope is a linear epitope. In some embodiments, the epitope is a conformational epitope. In some embodiments, the epitope is a neutralizing epitope site, for example, site I, II, or IV. In some embodiments, all neutralizing epitopes of the RSV F protein peptide or fragment thereof are present as the RSV viral antigen or immunogen.

In some cases, for example when the RSV viral antigen or immunogen is a fragment of an RSV F protein peptide, only a single subunit of the RSV F protein peptide is present.

In some embodiments, the RSV viral antigen or immunogen is or comprises an F1 subunit peptide. In some embodiments, the F1 subunit peptide is or comprises the amino acid sequence of 137-574 of a wildtype F protein. In some embodiments, the RSV viral antigen or immunogen is or comprises an F2 subunit peptide. In some embodiments, the RSV viral antigen or immunogen comprises an RSV F protein peptide containing a signal peptide, a heptad-repeat C (HRC) peptide, a pep27 peptide, a fusion peptide (FP), a heptad-repeat A (HRA) peptide, a Domain I peptide, a Domain II peptide, or heptad-repeat B (HRB) peptide, or any combination thereof. In some embodiments, the RSV viral antigen or immunogen comprises an RSV F protein peptide containing a signal peptide. In some embodiments, the RSV viral antigen or immunogen comprises an RSV F protein peptide containing a pep27 peptide. In some embodiments, the RSV viral antigen or immunogen comprises an RSV F protein peptide containing a fusion peptide (FP) (also known as a fusion domain (FD)). In some embodiments, the RSV viral antigen or immunogen comprises an RSV F protein peptide containing a signal peptide, a pep27 peptide, and a fusion peptide (FP).

In some embodiments, the RSV viral antigen or immunogen comprises an RSV F protein peptide containing an F1 subunit and an F2 subunit of the F protein. In some embodiments, the RSV viral antigen or immunogen comprises an RSV F protein peptide containing an F1 subunit peptide and an F2 subunit peptide without a pep 27 peptide of the F protein. In some embodiments, the RSV viral antigen or immunogen comprises an RSV F protein peptide containing an F1 subunit peptide, an F2 subunit peptide, and a pep 27 peptide of the F protein. In some embodiments, the RSV viral antigen or immunogen comprises an RSV F protein peptide containing an F1 subunit peptide, an F2 subunit peptide, a pep 27 peptide, and an FP of the F protein.

In some cases, for example when the viral antigen or immunogen includes both an F1 subunit peptide and an F2 subunit peptide of the RSV F protein peptide, the F1 and F2 subunits are linked. In some embodiments, the F1 and F2 subunits are linked by a disulfide bond. In some embodiments, the F1 and F2 subunits are linked by an artificially introduced linker. In some embodiments, the F1 and F2 subunits are linked through a pep27 peptide. For example, in some embodiments, the orientation from N- to C-terminus is or comprises F2-pep27-F1. In some embodiments, the orientation from N- to C-terminus is or comprises F2-pep27-FP-F1 (F2-pep27-FD-F1). In some embodiments, the FP is considered a structural feature of the F1 subunit peptide.

In some cases, the RSV viral antigen or immunogen is an RSV F protein peptide that does not contain a transmembrane (TM) domain peptide. In some cases, the RSV F protein does not contain a cytoplasmic (CP) domain peptide. In some cases, the RSV F protein does not contain a TM domain peptide or a CP domain peptide.

In some embodiments, the RSV viral antigen or immunogen comprises an RSV F protein peptide containing a protease cleavage site. In some embodiments, the protease cleavage site is specific to cleavage by the protease furin. In some embodiments, the protease cleavage site is specific to cleavage by the protease trypsin. In some embodiments, the protease cleavage site is specific to cleavage by the protease factor Xa. In some embodiments, the protease cleavage site is specific to cleavage by the protease cathepsin L.

In some cases, the RSV viral antigen or immunogen comprises an RSV F protein peptide that does not contain a protease cleavage site. In some cases, the RSV viral antigen or immunogen comprises an RSV F protein peptide that does not contain a protease cleavage site that is specific to cleavage by the protease furin. In some cases, the RSV viral antigen or immunogen comprises an RSV F protein peptide that does not contain a protease cleavage site that is specific to cleavage by the protease trypsin. In some cases, the RSV viral antigen or immunogen comprises an RSV F protein peptide that does not contain a protease cleavage site that is specific to cleavage by the protease factor Xa. In some cases, the RSV viral antigen or immunogen comprises an RSV F protein peptide that does not contain a protease cleavage site that is specific to cleavage by the protease cathepsin L.

In some embodiments, the RSV viral antigen or immunogen comprises an RSV F protein peptide that is soluble. In some embodiments, the soluble RSV F protein peptide lacks a TM domain peptide and a CP domain peptide. In some embodiments, the soluble RSV F protein peptide does not bind to a lipid bilayer, such as a membrane or viral envelope.

In some embodiments, the RSV F protein peptide is produced from a nucleic acid sequence that has been codon optimized. In some embodiments, the RSV F protein peptide is produced from a nucleic acid sequence that has not been codon optimized.

In some embodiments, the RSV F protein peptide can comprise any F protein sequence known in the art, such as those disclosed in U.S. Pat. No. 10,017,543, which is incorporated herein by reference in its entireties for all purposes.

In some embodiments, the RSV viral antigen or immunogen is or comprises an RSV F protein peptide having the amino acid sequence of 1-520 of SEQ ID NO: 31 or 32. In some embodiments, the RSV viral antigen or immunogen is or comprises an RSV F protein peptide having the amino acid sequence of 26-520 of SEQ ID NO: 31 or 32.

In some embodiments, the RSV viral antigen or immunogen is or comprises a sequence of F2, a sequence of pep27, and a sequence of F1 (such as F2-pep27-F1). In some embodiments, the RSV viral antigen or immunogen comprises fusion peptide exposure and has a post-fusion conformation. In some embodiments, the RSV viral antigen or immunogen comprises a furin cleavage site mutation. In some embodiments, the RSV viral antigen or immunogen comprises a furin site I mutation (e.g., R109A) and/or a furin site II mutation (e.g., R136A), and in some of these examples, the RSV viral antigen or immunogen has a post-fusion conformation while in other examples, the RSV viral antigen or immunogen has a pre-fusion conformation. In some embodiments, the RSV viral antigen or immunogen comprises a furin site I mutation and a furin site II mutation (e.g., R109A/R136A), and in some of these examples, the RSV viral antigen or immunogen comprises a full-length F0 without fusion peptide exposure and has a pre-fusion conformation. In some embodiments, the RSV viral antigen or immunogen comprises one or more mutations that prevent the formation of the long helix and/or stabilize the α4-α5 hinge loop. In some embodiments, the RSV viral antigen or immunogen comprises one or more mutations that preserve pre-fusion conformation. In some embodiments, the RSV viral antigen or immunogen comprises one or more mutations that improve expression. In some embodiments, substitution of position 161, 182 and 215 (e.g., with proline) resulted in higher expression levels, and E161P and S215P also increased protein stability. In some embodiments, the RSV viral antigen or immunogen comprises E161P and/or S215P and has a pre-fusion conformation. In some embodiments, the RSV viral antigen or immunogen comprises R109A, R136A, E161P and/or S215P and has a pre-fusion conformation.

In some embodiments, the viral antigen or immunogen comprises the sequence set forth in SEQ ID NO: 17. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 17, including a sequence comprising substitution, deletion, and/or insertion at one or more amino acid positions.

In some embodiments, the viral antigen or immunogen comprises the sequence set forth in SEQ ID NO: 18. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 18, including a sequence comprising substitution, deletion, and/or insertion at one or more amino acid positions.

In some embodiments, the viral antigen or immunogen comprises the sequence set forth in SEQ ID NO: 19. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 19, including a sequence comprising substitution, deletion, and/or insertion at one or more amino acid positions.

In some embodiments, the viral antigen or immunogen comprises the sequence set forth in SEQ ID NO: 20. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 20, including a sequence comprising substitution, deletion, and/or insertion at one or more amino acid positions.

In some embodiments, the viral antigen or immunogen comprises the sequence set forth in SEQ ID NO: 21. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 21, including a sequence comprising substitution, deletion, and/or insertion at one or more amino acid positions.

In some embodiments, the viral antigen or immunogen comprises the sequence set forth in SEQ ID NO: 22. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 22, including a sequence comprising substitution, deletion, and/or insertion at one or more amino acid positions.

In some embodiments, the viral antigen or immunogen comprises the sequence set forth in SEQ ID NO: 23. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 23, including a sequence comprising substitution, deletion, and/or insertion at one or more amino acid positions.

In some embodiments, the viral antigen or immunogen comprises the sequence set forth in SEQ ID NO: 24. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 24, including a sequence comprising substitution, deletion, and/or insertion at one or more amino acid positions.

In some embodiments, the viral antigen or immunogen comprises the sequence set forth in SEQ ID NO: 25. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 25, including a sequence comprising substitution, deletion, and/or insertion at one or more amino acid positions.

In some embodiments, the viral antigen or immunogen comprises the sequence set forth in SEQ ID NO: 26. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 26, including a sequence comprising substitution, deletion, and/or insertion at one or more amino acid positions.

In some embodiments, the viral antigen or immunogen comprises the sequence set forth in SEQ ID NO: 27. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 27, including a sequence comprising substitution, deletion, and/or insertion at one or more amino acid positions.

In some embodiments, the viral antigen or immunogen comprises the sequence set forth in SEQ ID NO: 28. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 28, including a sequence comprising substitution, deletion, and/or insertion at one or more amino acid positions.

In some embodiments, the viral antigen or immunogen comprises the sequence set forth in SEQ ID NO: 29. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 29, including a sequence comprising substitution, deletion, and/or insertion at one or more amino acid positions.

In some embodiments, the viral antigen or immunogen comprises the sequence set forth in SEQ ID NO: 30. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 30, including a sequence comprising substitution, deletion, and/or insertion at one or more amino acid positions.

In some embodiments, the viral antigen or immunogen comprises the sequence set forth in SEQ ID NO: 31. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 31, including a sequence comprising substitution, deletion, and/or insertion at one or more amino acid positions.

In some embodiments, the viral antigen or immunogen comprises the sequence set forth in SEQ ID NO: 32. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 32, including a sequence comprising substitution, deletion, and/or insertion at one or more amino acid positions.

In some embodiments, the viral antigen or immunogen comprises the sequence set forth in SEQ ID NO: 33. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 33, including a sequence comprising substitution, deletion, and/or insertion at one or more amino acid positions.

In some embodiments, the viral antigen or immunogen comprises the sequence set forth in SEQ ID NO: 34. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 34, including a sequence comprising substitution, deletion, and/or insertion at one or more amino acid positions.

In some embodiments, the viral antigen or immunogen comprises the sequence set forth in SEQ ID NO: 35. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 35, including a sequence comprising substitution, deletion, and/or insertion at one or more amino acid positions.

In some embodiments, the viral antigen or immunogen comprises the sequence set forth in SEQ ID NO: 36. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 36, including a sequence comprising substitution, deletion, and/or insertion at one or more amino acid positions.

In some embodiments, the viral antigen or immunogen comprises the sequence set forth in SEQ ID NO: 37. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 37, including a sequence comprising substitution, deletion, and/or insertion at one or more amino acid positions.

In some embodiments, the viral antigen or immunogen comprises the sequence set forth in SEQ ID NO: 38. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 38, including a sequence comprising substitution, deletion, and/or insertion at one or more amino acid positions.

In some embodiments, the viral antigen or immunogen comprises the sequence set forth in SEQ ID NO: 39. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 39, including a sequence comprising substitution, deletion, and/or insertion at one or more amino acid positions.

In some embodiments, the viral antigen or immunogen comprises the sequence set forth in SEQ ID NO: 40. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 40, including a sequence comprising substitution, deletion, and/or insertion at one or more amino acid positions.

In some embodiments, the viral antigen or immunogen comprises the sequence set forth in SEQ ID NO: 41. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 41, including a sequence comprising substitution, deletion, and/or insertion at one or more amino acid positions.

In some embodiments, the viral antigen or immunogen comprises the sequence set forth in SEQ ID NO: 42. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 42, including a sequence comprising substitution, deletion, and/or insertion at one or more amino acid positions.

In some embodiments, the viral antigen or immunogen comprises the sequence set forth in SEQ ID NO: 43. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 43, including a sequence comprising substitution, deletion, and/or insertion at one or more amino acid positions.

In some embodiments, the viral antigen or immunogen comprises the sequence set forth in SEQ ID NO: 44. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 44, including a sequence comprising substitution, deletion, and/or insertion at one or more amino acid positions.

In some embodiments, the viral antigen or immunogen comprises the sequence set forth in SEQ ID NO: 45. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 45, including a sequence comprising substitution, deletion, and/or insertion at one or more amino acid positions.

In some embodiments, the viral antigen or immunogen comprises the sequence set forth in SEQ ID NO: 46. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 46, including a sequence comprising substitution, deletion, and/or insertion at one or more amino acid positions.

In some embodiments, the viral antigen or immunogen comprises the sequence set forth in SEQ ID NO: 47. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 47, including a sequence comprising substitution, deletion, and/or insertion at one or more amino acid positions.

In some embodiments, the viral antigen or immunogen herein may comprises an RSV glycoprotein (G) or fragment, variant, or mutant thereof; an RSV small hydrophobic protein (SH) or fragment, variant, or mutant thereof; an RSV fusion protein (F) or fragment, variant, or mutant thereof; an RSV matrix protein (M) or fragment, variant, or mutant thereof; an RSV nucleoprotein (N) or fragment, variant, or mutant thereof; an RSV phosphoprotein (P) or fragment, variant, or mutant thereof; an RSV “large” protein (L) or fragment, variant, or mutant thereof; an M2-1 protein or fragment, variant, or mutant thereof; an RSV M2-2 protein or fragment, variant, or mutant thereof; an RSV NS-1 protein or fragment, variant, or mutant thereof; or an RSV Ns-2 protein or fragment, variant, or mutant thereof; or any combination thereof.

In some embodiments, the viral antigen or immunogen is produced from a nucleic acid sequence that has been codon optimized. In some embodiments, the viral antigen or immunogen is produced from a nucleic acid sequence that has not been codon optimized.

In some embodiments, the RSV viral antigen or immunogen as referred to herein can include recombinant polypeptides or fusion peptides comprising said viral antigen or immunogen. The terms viral antigen or immunogen may be used to refer to proteins comprising an RSV viral antigen or immunogen. In certain cases, the RSV viral antigen or immunogen is an RSV protein peptide as provided herein.

### II. Recombinant Peptides and Proteins

It is contemplated that the RSV viral antigens and immunogens provided herein, e.g., RSV F protein peptides (see, Section I), can be combined, e.g., linked, to other proteins or peptides to form recombinant polypeptides, including fusion peptides. In some embodiments, individual recombinant polypeptides (e.g., monomers) provided herein associate to form multimers, e.g., trimers, of recombinant polypeptides. In some embodiments, association of the individual recombinant polypeptide monomers occurs via covalent interactions. In some embodiments, association of the individual recombinant polypeptide monomers occurs via non-covalent interactions. In some embodiments, the interaction, e.g., covalent or non-covalent, is effected by the protein or peptide to which the RSV viral antigen or immunogen, e.g., RSV F protein peptide, is linked. In some embodiments, for example when the RSV viral antigen or immunogen is an RSV F protein peptide as described herein, the protein or peptide to which it will be linked can be selected such that the native homotrimeric structure of the glycoprotein is preserved. This can be advantageous for evoking a strong and effective immunogenic response to the RSV F protein peptide. For example, preservation and/or maintenance of the native conformation of the RSV viral antigens or immunogens (e.g., RSV F protein peptide) may improve or allow access to antigenic sites capable to generating an immune response. In some cases, the recombinant polypeptide comprising an RSV F protein peptide described herein, e.g., see Section I, is referred to herein alternatively as a recombinant RSV F antigen, recombinant RSV F immunogen, or a recombinant RSV F protein.

It is further contemplated that in some cases, the recombinant polypeptides or multimerized recombinant polypeptides thereof aggregate or can be aggregated to form a protein comprising a plurality of RSV viral antigen and/or immunogen recombinant polypeptides. Formation of such proteins may be advantageous for generating a strong and effective immunogenic response to the RSV viral antigens and/or immunogens. For instance, formation of a protein comprising a plurality of recombinant polypeptides, and thus a plurality of RSV viral antigens, e.g., RSV F protein peptides, may preserve the tertiary and/or quaternary structures of the viral antigen, allowing an immune response to be mounted against the native structure. In some cases, the aggregation may confer structural stability of the RSV viral antigen or immunogen, which in turn can afford access to potentially antigenic sites capable of promoting an immune response.

### 1. Fusion Peptides and Recombinant Polypeptides

In some embodiments, the RSV viral antigen or immunogen can be linked at their C-terminus (C-terminal linkage) to a trimerization domain to promote trimerization of the monomers. In some embodiments, the trimerization stabilizes the membrane proximal aspect of the RSV viral antigen or immunogen, e.g., RSV F protein peptide, in a trimeric configuration.

Non-limiting examples of exogenous multimerization domains that promote stable trimers of soluble recombinant proteins include: the GCN4 leucine zipper (Harbury et al. 1993 Science 262:1401-1407), the trimerization motif from the lung surfactant protein (Hoppe et al. 1994 FEBS Lett 344:191-195), collagen (McAlinden et al. 2003 J Biol Chem 278:42200-42207), and the phage T4 fibritin Foldon (Miroshnikov et al. 1998 Protein Eng 11:329-414), any of which can be linked to an RSV viral antigen or immunogen described herein (e.g., by linkage to the C-terminus of an RSV F peptide) to promote trimerization of the recombinant viral antigen or immunogen. See also U.S. Pat. Nos. 7,268,116, 7,666,837, 7,691,815, 10,618,949, 10,906,944, and 10,960,070, and US 2020/0009244, which are incorporated herein by reference in their entireties for all purposes.

In some embodiments, one or more peptide linkers (such as a gly-ser linker, for example, a 10 amino acid glycine-serine peptide linker) can be used to link the recombinant viral antigen or immunogen to the multimerization domain. The trimer can include any of the stabilizing mutations provided herein (or combinations thereof) as long as the recombinant viral antigen or immunogen trimer retains the desired properties (e.g., the prefusion conformation).

To be therapeutically feasible, a desired trimerizing protein moiety for biologic drug designs should satisfy the following criteria. Ideally it should be part of a naturally secreted protein, like immunoglobulin Fc, that is also abundant (non-toxic) in the circulation, human in origin (lack of immunogenicity), relatively stable (long half-life) and capable of efficient self-trimerization which is strengthened by inter-chain covalent disulfide bonds so the trimerized RSV viral antigens or immunogens are structurally stable.

Collagen is a family of fibrous proteins that are the major components of the extracellular matrix. It is the most abundant protein in mammals, constituting nearly 25% of the total protein in the body. Collagen plays a major structural role in the formation of bone, tendon, skin, cornea, cartilage, blood vessels, and teeth. The fibrillar types of collagen I, H, III, IV, V, and XI are all synthesized as larger trimeric precursors, called procollagens, in which the central uninterrupted triple-helical domain consisting of hundreds of “G-X-Y” repeats (or glycine repeats) is flanked by non-collagenous domains (NC), the N-propeptide and the C-propeptide. Both the C- and N-terminal extensions are processed proteolytically upon secretion of the procollagen, an event that triggers the assembly of the mature protein into collagen fibrils which forms an insoluble cell matrix. BMP-1 is a protease that recognizes a specific peptide sequence of procollagen near the junction between the glycine repeats and the C-prodomain of collagens and is responsible for the removal of the propeptide. The shed trimeric C-propeptide of type I collagen is found in human sera of normal adults at a concentration in the range of 50-300 ng/mL, with children having a much higher level which is indicative of active bone formation. In people with familial high serum concentration of C-propeptide of type 1 collagen, the level could reach as high as 1-6 μg/mL with no apparent abnormality, suggesting the C-propeptide is not toxic. Structural study of the trimeric C-propeptide of collagen suggested that it is a tri-lobed structure with all three subunits coming together in a junction region near their N-termini to connect to the rest of the procollagen molecule. Such geometry in projecting proteins to be fused in one direction is similar to that of Fc dimer.

Type I, IV, V and XI collagens are mainly assembled into heterotrimeric forms consisting of either two α-1 chains and one α-2 chain (for Type I, IV, V), or three different a chains (for Type XI), which are highly homologous in sequence. The type II and III collagens are both homotrimers of α-1 chain. For type I collagen, the most abundant form of collagen, stable α(I) homotrimer is also formed and is present at variable levels in different tissues. Most of these collagen C-propeptide chains can self-assemble into homotrimers, when over-expressed alone in a cell. Although the N-propeptide domains are synthesized first, molecular assembly into trimeric collagen begins with the in-register association of the C-propeptides. It is believed the C-propeptide complex is stabilized by the formation of interchain disulfide bonds, but the necessity of disulfide bond formation for proper chain registration is not clear. The triple helix of the glycine repeats and is then propagated from the associated C-termini to the N-termini in a zipper-like manner. This knowledge has led to the creation of non-natural types of collagen matrix by swapping the C-propeptides of different collagen chains using recombinant DNA technology. Non-collagenous proteins, such as cytokines and growth factors, also have been fused to the N-termini of either pro-collagens or mature collagens to allow new collagen matrix formation, which is intended to allow slow release of the noncollagenous proteins from the cell matrix. However, under both circumstances, the C-propeptides are required to be cleaved before recombinant collagen fibril assembly into an insoluble cell matrix.

Although, other protein trimerization domains, such as those from GCN4 from yeast fibritin from bacteria phage T4 and aspartate transcarbamoylase of Escherichia coli, have been described previously to allow trimerization of heterologous proteins, none of these trimerizing proteins are human in nature, nor are they naturally secreted proteins. As such, any trimeric fusion proteins would have to be made intracellularly, which not only may fold incorrectly for naturally secreted proteins such as soluble receptors, but also make purification of such fusion proteins from thousands of other intracellular proteins difficult. Moreover, the fatal drawback of using such non-human protein trimerization domains (e.g. from yeast, bacteria phage and bacteria) for trimeric biologic drug design is their presumed immunogenicity in the human body, rendering such fusion proteins ineffective shortly after injecting them into the human body.

The use of collagen in a recombinant polypeptide as described herein thus has many advantages, including: (1) collagen is the most abundant protein secreted in the body of a mammal, constituting nearly 25% of the total proteins in the body; (2) the major forms of collagen naturally occur as trimeric helixes, with their globular C-propeptides being responsible for the initiating of trimerization; (3) the trimeric C-propeptide of collagen proteolytically released from the mature collagen is found naturally at sub microgram/mL level in the blood of mammals and is not known to be toxic to the body; (4) the linear triple helical region of collagen can be included as a linker with predicted 2.9 Å spacing per residue, or excluded as part of the fusion protein so the distance between a protein to be trimerized and the C-propeptide of collagen can be precisely adjusted to achieve an optimal biological activity; (5) the recognition site of BMP1 which cleaves the C-propeptide off the pro-collagen can be mutated or deleted to prevent the disruption of a trimeric fusion protein; (6) the C-propeptide domain self-trimerizes via disulfide bonds and it provides a universal affinity tag, which can be used for purification of any secreted fusion proteins created. In some embodiments, the C-propeptide of collagen to which the RSV viral antigen and immunogen, e.g., RSV F protein peptide, enables the recombinant production of soluble, covalently-linked homotrimeric fusion proteins.

In some embodiments, the RSV viral antigen or immunogen is linked to a C-terminal propeptide of collagen to form a recombinant polypeptide. In some embodiments, the C-terminal propeptides of the recombinant polypeptides form inter-polypeptide disulfide bonds. In some embodiments, the recombinant proteins form trimers. In some embodiments, the RSV viral antigen or immunogen is an RSV F protein peptide as described in Section I.

In some embodiments, the C-terminal propeptide is of human collagen. In some embodiments, the C-terminal propeptide comprises a C-terminal polypeptide of proα1(I), proαI(II), proα1(III), proα1(V), proα1(XI), proα2(I), proα2(V), proα2(XI), or proα3(XI), or a fragment thereof. In some embodiments, the C-terminal propeptide is or comprises a C-terminal polypeptide of proα1(I).

In some embodiments, the C-terminal propeptide is or comprises the amino acid sequence set forth by SEQ ID NO: 48. In some embodiments, the C-terminal propeptide is an amino acid sequence having at least or about 85%, 90%6, 92%, 95%, or 97% sequence identity to sequence of SEQ ID NO: 48. In some embodiments, the C-terminal propeptide is or comprises the amino acid sequence set forth by SEQ ID NO: 49. In some embodiments, the C-terminal propeptide is an amino acid sequence having at least or about 85%, 90%, 92%, 95%, or 97% sequence identity to sequence of SEQ ID NO: 49. In some embodiments, the C-terminal propeptide is or is the amino acid sequence set forth by SEQ ID NO: 50. In some embodiments, the C-terminal propeptide exhibits an amino acid sequence having at least or about 85%, 90%, 92%, 95%, or 97% sequence identity to sequence of SEQ ID NO: 50. In some embodiments, the C-terminal propeptide is or comprises the amino acid sequence set forth by SEQ ID NO: 51. In some embodiments, the C-terminal propeptide is an amino acid sequence having at least or about 85%, 90%, 92%, 95%, or 97% sequence identity to sequence of SEQ ID NO: 51. In some embodiments, the C-terminal propeptide is or comprises the amino acid sequence set forth by SEQ ID NO: 52. In some embodiments, the C-terminal propeptide is an amino acid sequence having at least or about 85%, 90%, 92%, 95%, or 97% sequence identity to sequence of SEQ ID NO: 52. In some embodiments, the C-terminal propeptide is or comprises the amino acid sequence set forth by SEQ ID NO: 53. In some embodiments, the C-terminal propeptide is an amino acid sequence having at least or about 85%, 90%, 92%, 95%, or 97% sequence identity to sequence of SEQ ID NO: 53.

In some embodiments, the C-terminal propeptide is or comprises the amino acid sequence set forth by SEQ ID NO: 54. In some embodiments, the C-terminal propeptide is an amino acid sequence having at least or about 85%, 90%, 92%, 95%, or 97% sequence identity to sequence of SEQ ID NO: 54. In some embodiments, the C-terminal propeptide is or comprises the amino acid sequence set forth by SEQ ID NO: 55. In some embodiments, the C-terminal propeptide is an amino acid sequence having at least or about 85%, 90%, 92%, 95%, or 97% sequence identity to sequence of SEQ ID NO: 55. In some embodiments, the C-terminal propeptide is or comprises the amino acid sequence set forth by SEQ ID NO: 56. In some embodiments, the C-terminal propeptide is an amino acid sequence having at least or about 85%, 90%, 92%, 95%, or 97% sequence identity to sequence of SEQ ID NO: 56. In some embodiments, the C-terminal propeptide is or comprises the amino acid sequence set forth by SEQ ID NO: 57. In some embodiments, the C-terminal propeptide is an amino acid sequence having at least or about 85%, 90%, 92%, 95%, or 97% sequence identity to sequence of SEQ ID NO: 57. In some embodiments, the C-terminal propeptide is or comprises the amino acid sequence set forth by SEQ ID NO: 58. In some embodiments, the C-terminal propeptide is an amino acid sequence having at least or about 85%, 90%, 92%, 95%, or 97% sequence identity to sequence of SEQ ID NO: 58. In some embodiments, the C-terminal propeptide is or comprises the amino acid sequence set forth by SEQ ID NO: 59. In some embodiments, the C-terminal propeptide is an amino acid sequence having at least or about 85%, 90%, 92%, 95%, or 97% sequence identity to sequence of SEQ ID NO: 59.

In some embodiments, the C-terminal propeptide is or comprises the amino acid sequence set forth by SEQ ID NO: 60. In some embodiments, the C-terminal propeptide is an amino acid sequence having at least or about 85%, 90%, 92%, 95%, or 97% sequence identity to sequence of SEQ ID NO: 60. In some embodiments, the C-terminal propeptide is or comprises the amino acid sequence set forth by SEQ ID NO: 61. In some embodiments, the C-terminal propeptide is an amino acid sequence having at least or about 85%, 90%, 92%, 95%, or 97% sequence identity to sequence of SEQ ID NO: 61. In some embodiments, the C-terminal propeptide is or comprises the amino acid sequence set forth by SEQ ID NO: 62. In some embodiments, the C-terminal propeptide is an amino acid sequence having at least or about 85%, 90%, 92%, 95%, or 97% sequence identity to sequence of SEQ ID NO: 62. In some embodiments, the C-terminal propeptide is or comprises the amino acid sequence set forth by SEQ ID NO: 63. In some embodiments, the C-terminal propeptide is an amino acid sequence having at least or about 85%, 90%, 92%, 95%, or 97% sequence identity to sequence of SEQ ID NO: 63.

In some embodiments, the C-terminal propeptide is or comprises the amino acid sequence of a collagen trimerization domain (e.g., C-propeptide of human α1(I) collagen) with an aspartic acid (D) to asparagine (N) substitution in the BMP-1 site, for instance where RAD is mutated to RAN. In some embodiments, the C-terminal propeptide is or comprises the amino acid sequence of a collagen trimerization domain (e.g., C-propeptide of human α1(I) collagen) with an alanine (A) to asparagine (N) substitution in the BMP-1 site, for instance where RAD is mutated to RND. In some embodiments, the C-terminal propeptide herein may comprise a mutated BMP-1 site, e.g., RSAN instead of DDAN. In some embodiments, the C-terminal propeptide herein may comprise a BMP-1 site, e.g., a sequence comprising the RAD (e.g., RADDAN) sequence instead of RAN (e.g., RANDAN) or RND (e.g., RNDDAN) may be used in a fusion polypeptide disclosed herein.

In some embodiments, the C-terminal propeptide is or comprises an amino acid sequence that is a fragment of any of SEQ ID NOs: 48-63.

In some embodiments, the C-terminal propeptide can comprise a sequence comprising glycine-X-Y repeats, wherein X and Y are independently any amino acid, or an amino acid sequence at least 85%, 90%, 92%, 95%, or 97% identical thereto capable of forming inter-polypeptide disulfide bonds and trimerizing the recombinant polypeptides. In some embodiments, X and Y are independently proline or hydroxyproline.

In some cases where an RSV F peptide protein (e.g., RSV viral antigen or immunogen, e.g., see, Section I) is linked to the C-terminal propeptide to form the recombinant polypeptide, the recombinant polypeptides form a trimer resulting in a homotrimer of RSV F protein peptides. In some embodiments, the trimerized recombinant polypeptides contain F protein peptide trimers as crutch-shaped rods. In some embodiments, the RSV F protein peptides of the trimerized recombinant polypetides are in a prefusion conformation. In some embodiments, the RSV F protein peptides of the trimerized recombinant polypetides are in a postfusion conformation. In some embodiments, the confirmation state allows for access to different antigenic sites on the F protein peptides. In some embodiments, the antigenic sites are epitopes, such as linear epitopes or conformational epitopes. An advantage of having a trimerized recombinant polypeptides as described is that an immune response can be mounted against a variety of potential and diverse antigenic sites.

In some embodiments, trimerized recombinant polypeptides include individual recombinant polypeptides comprising the same viral antigen or immunogen. In some embodiments, trimerized recombinant polypeptides include individual recombinant polypeptides each comprising a different viral antigen or immunogen from the other recombinant polypeptides. In some embodiments, trimerized recombinant polypeptides include individual recombinant polypeptides wherein one of the individual recombinant polypeptides comprises a viral antigen or immunogen different from the other recombinant polypeptides. In some embodiments, trimerized recombinant polypeptides include individual recombinant polypeptides wherein two of the individual recombinant polypeptides comprise the same viral antigen or immunogen, and the viral antigen or immunogen is different from the viral antigen or immunogen comprised in the remaining recombinant polypeptide.

In some embodiments, the recombinant polypeptide comprises any RSV viral antigen or immunogen described in Section I. In some embodiments, the recombinant polypeptide comprises any RSV viral antigen or immunogen described in Section I linked, as described herein, to the C-terminal propeptide of collagen as described herein.

In some embodiments, the recombinant polypeptide or the fusion protein comprises a first sequence set forth in any of SEQ ID NOs: 17-47 linked to a second sequence set forth in any of SEQ ID NOs: 48-63, wherein the C terminus of the first sequence is directly linked to the N terminus of the second sequence.

In some embodiments, the recombinant polypeptide or the fusion protein comprises a first sequence set forth in any of SEQ ID NOs: 17-47 linked to a second sequence set forth in any of SEQ ID NOs: 48-63, wherein the C terminus of the first sequence is indirectly linked to the N terminus of the second sequence, e.g. through a linker. In some embodiments, the linker comprises a sequence comprising glycine-X-Y repeats.

In some embodiments, the recombinant polypeptide is or comprises the sequence set forth in SEQ ID NO: 1. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 1, including a sequence comprising substitution, deletion, and/or insertion at one or more amino acid positions, such as 102, 106, 107, 108, 109, 131, 132, 133, 134, 135, 136, 137, 138, 139, 140, 141, 142, 143, 144, 145, 146, 147, 148, 149, 150, 151, 152, 153, 154, 155, 161, 215, 218, 379, or 447 (amino acid positions with respect to SEQ ID NO: 31 or 32), or any combination thereof. In some embodiments, the recombinant polypeptide is or comprises a variant of SEQ ID NO: 1 and the variant comprises any one, two, three, four, five or more of the mutations selected from the group consisting of P102A, R109A, R136A, E161P, E218A, S215P, 1379A, and M447V, or any combination thereof.

In some embodiments, the recombinant polypeptide is or comprises the sequence set forth in SEQ ID NO: 2. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 2, including a sequence comprising substitution, deletion, and/or insertion at one or more amino acid positions, such as 102, 106, 107, 108, 109, 131, 132, 133, 134, 135, 136, 137, 138, 139, 140, 141, 142, 143, 144, 145, 146, 147, 148, 149, 150, 151, 152, 153, 154, 155, 161, 215, 218, 379, or 447 (amino acid positions with respect to SEQ ID NO: 31 or 32), or any combination thereof. In some embodiments, the recombinant polypeptide is or comprises a variant of SEQ ID NO: 2 and the variant comprises any one, two, three, four, five or more of the mutations selected from the group consisting of P102A, R109A, R136A, E161P, E218A, S215P, 1379A, and M447V, or any combination thereof.

In some embodiments, the recombinant polypeptide is or comprises the sequence set forth in SEQ ID NO: 3. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 3, including a sequence comprising substitution, deletion, and/or insertion at one or more amino acid positions, such as 102, 106, 107, 108, 109, 131, 132, 133, 134, 135, 136, 137, 138, 139, 140, 141, 142, 143, 144, 145, 146, 147, 148, 149, 150, 151, 152, 153, 154, 155, 161, 215, 218, 379, or 447 (amino acid positions with respect to SEQ ID NO: 31 or 32), or any combination thereof. In some embodiments, the recombinant polypeptide is or comprises a variant of SEQ ID NO: 3 and the variant comprises any one, two, three, four, five or more of the mutations selected from the group consisting of P102A, R109A, R136A, E161P, E218A, S215P, 1379A, and M447V, or any combination thereof.

In some embodiments, the recombinant polypeptide is or comprises the sequence set forth in SEQ ID NO: 4. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 4, including a sequence comprising substitution, deletion, and/or insertion at one or more amino acid positions, such as 102, 106, 107, 108, 109, 131, 132, 133, 134, 135, 136, 137, 138, 139, 140, 141, 142, 143, 144, 145, 146, 147, 148, 149, 150, 151, 152, 153, 154, 155, 161, 215, 218, 379, or 447 (amino acid positions with respect to SEQ ID NO: 31 or 32), or any combination thereof. In some embodiments, the recombinant polypeptide is or comprises a variant of SEQ ID NO: 4 and the variant comprises any one, two, three, four, five or more of the mutations selected from the group consisting of P102A, R109A, R136A, E161P, E218A, S215P, 1379A, and M447V, or any combination thereof.

In some embodiments, the recombinant polypeptide is or comprises the sequence set forth in SEQ ID NO: 5. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 5, including a sequence comprising substitution, deletion, and/or insertion at one or more amino acid positions, such as 102, 106, 107, 108, 109, 131, 132, 133, 134, 135, 136, 137, 138, 139, 140, 141, 142, 143, 144, 145, 146, 147, 148, 149, 150, 151, 152, 153, 154, 155, 161, 215, 218, 379, or 447 (amino acid positions with respect to SEQ ID NO: 31 or 32), or any combination thereof. In some embodiments, the recombinant polypeptide is or comprises a variant of SEQ ID NO: 5 and the variant comprises any one, two, three, four, five or more of the mutations selected from the group consisting of P102A, R109A, R136A, E161P, E218A, S215P, 1379A, and M447V, or any combination thereof.

In some embodiments, the recombinant polypeptide is or comprises the sequence set forth in SEQ ID NO: 6. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 6, including a sequence comprising substitution, deletion, and/or insertion at one or more amino acid positions, such as 102, 106, 107, 108, 109, 131, 132, 133, 134, 135, 136, 137, 138, 139, 140, 141, 142, 143, 144, 145, 146, 147, 148, 149, 150, 151, 152, 153, 154, 155, 161, 215, 218, 379, or 447 (amino acid positions with respect to SEQ ID NO: 31 or 32), or any combination thereof. In some embodiments, the recombinant polypeptide is or comprises a variant of SEQ ID NO: 6 and the variant comprises any one, two, three, four, five or more of the mutations selected from the group consisting of P102A, R109A, R136A, E161P, E218A, S215P, 1379A, and M447V, or any combination thereof.

In some embodiments, the recombinant polypeptide is or comprises the sequence set forth in SEQ ID NO: 7. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 7, including a sequence comprising substitution, deletion, and/or insertion at one or more amino acid positions, such as 102, 106, 107, 108, 109, 131, 132, 133, 134, 135, 136, 137, 138, 139, 140, 141, 142, 143, 144, 145, 146, 147, 148, 149, 150, 151, 152, 153, 154, 155, 161, 215, 218, 379, or 447 (amino acid positions with respect to SEQ ID NO: 31 or 32), or any combination thereof. In some embodiments, the recombinant polypeptide is or comprises a variant of SEQ ID NO: 7 and the variant comprises any one, two, three, four, five or more of the mutations selected from the group consisting of P102A, R109A, R136A, E161P, E218A, S215P, 1379A, and M447V, or any combination thereof.

In some embodiments, the recombinant polypeptide is or comprises the sequence set forth in SEQ ID NO: 8. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 8, including a sequence comprising substitution, deletion, and/or insertion at one or more amino acid positions, such as 102, 106, 107, 108, 109, 131, 132, 133, 134, 135, 136, 137, 138, 139, 140, 141, 142, 143, 144, 145, 146, 147, 148, 149, 150, 151, 152, 153, 154, 155, 161, 215, 218, 379, or 447 (amino acid positions with respect to SEQ ID NO: 31 or 32), or any combination thereof. In some embodiments, the recombinant polypeptide is or comprises a variant of SEQ ID NO: 8 and the variant comprises any one, two, three, four, five or more of the mutations selected from the group consisting of P102A, R109A, R136A, E161P, E218A, S215P, 1379A, and M447V, or any combination thereof.

In some embodiments, the recombinant polypeptide is or comprises the sequence set forth in SEQ ID NO: 9. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 9, including a sequence comprising substitution, deletion, and/or insertion at one or more amino acid positions, such as 102, 106, 107, 108, 109, 131, 132, 133, 134, 135, 136, 137, 138, 139, 140, 141, 142, 143, 144, 145, 146, 147, 148, 149, 150, 151, 152, 153, 154, 155, 161, 215, 218, 379, or 447 (amino acid positions with respect to SEQ ID NO: 31 or 32), or any combination thereof. In some embodiments, the recombinant polypeptide is or comprises a variant of SEQ ID NO: 9 and the variant comprises any one, two, three, four, five or more of the mutations selected from the group consisting of P102A, R109A, R136A, E161P, E218A, S215P, 1379A, and M447V, or any combination thereof.

In some embodiments, the recombinant polypeptide is or comprises the sequence set forth in SEQ ID NO: 10. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 10, including a sequence comprising substitution, deletion, and/or insertion at one or more amino acid positions, such as 102, 106, 107, 108, 109, 131, 132, 133, 134, 135, 136, 137, 138, 139, 140, 141, 142, 143, 144, 145, 146, 147, 148, 149, 150, 151, 152, 153, 154, 155, 161, 215, 218, 379, or 447 (amino acid positions with respect to SEQ ID NO: 31 or 32), or any combination thereof. In some embodiments, the recombinant polypeptide is or comprises a variant of SEQ ID NO: 10 and the variant comprises any one, two, three, four, five or more of the mutations selected from the group consisting of P102A, R109A, R136A, E161P, E218A, S215P, 1379A, and M447V, or any combination thereof.

In some embodiments, the recombinant polypeptide is or comprises the sequence set forth in SEQ ID NO: 11. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 11, including a sequence comprising substitution, deletion, and/or insertion at one or more amino acid positions, such as 102, 106, 107, 108, 109, 131, 132, 133, 134, 135, 136, 137, 138, 139, 140, 141, 142, 143, 144, 145, 146, 147, 148, 149, 150, 151, 152, 153, 154, 155, 161, 215, 218, 379, or 447 (amino acid positions with respect to SEQ ID NO: 31 or 32), or any combination thereof. In some embodiments, the recombinant polypeptide is or comprises a variant of SEQ ID NO: 11 and the variant comprises any one, two, three, four, five or more of the mutations selected from the group consisting of P102A, R109A, R136A, E161P, E218A, S215P, 1379A, and M447V, or any combination thereof.

In some embodiments, the recombinant polypeptide is or comprises the sequence set forth in SEQ ID NO: 12. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 12, including a sequence comprising substitution, deletion, and/or insertion at one or more amino acid positions, such as 102, 106, 107, 108, 109, 131, 132, 133, 134, 135, 136, 137, 138, 139, 140, 141, 142, 143, 144, 145, 146, 147, 148, 149, 150, 151, 152, 153, 154, 155, 161, 215, 218, 379, or 447 (amino acid positions with respect to SEQ ID NO: 31 or 32), or any combination thereof. In some embodiments, the recombinant polypeptide is or comprises a variant of SEQ ID NO: 12 and the variant comprises any one, two, three, four, five or more of the mutations selected from the group consisting of P102A, R109A, R136A, E161P, E218A, S215P, 1379A, and M447V, or any combination thereof.

In some embodiments, the recombinant polypeptide is or comprises the sequence set forth in SEQ ID NO: 13. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 13, including a sequence comprising substitution, deletion, and/or insertion at one or more amino acid positions, such as 102, 106, 107, 108, 109, 131, 132, 133, 134, 135, 136, 137, 138, 139, 140, 141, 142, 143, 144, 145, 146, 147, 148, 149, 150, 151, 152, 153, 154, 155, 161, 215, 218, 379, or 447 (amino acid positions with respect to SEQ ID NO: 31 or 32), or any combination thereof. In some embodiments, the recombinant polypeptide is or comprises a variant of SEQ ID NO: 13 and the variant comprises any one, two, three, four, five or more of the mutations selected from the group consisting of P102A, R109A, R136A, E161P, E218A, S215P, 1379A, and M447V, or any combination thereof.

In some embodiments, the recombinant polypeptide is or comprises the sequence set forth in SEQ ID NO: 14. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 14, including a sequence comprising substitution, deletion, and/or insertion at one or more amino acid positions, such as 102, 106, 107, 108, 109, 131, 132, 133, 134, 135, 136, 137, 138, 139, 140, 141, 142, 143, 144, 145, 146, 147, 148, 149, 150, 151, 152, 153, 154, 155, 161, 215, 218, 379, or 447 (amino acid positions with respect to SEQ ID NO: 31 or 32), or any combination thereof. In some embodiments, the recombinant polypeptide is or comprises a variant of SEQ ID NO: 14 and the variant comprises any one, two, three, four, five or more of the mutations selected from the group consisting of P102A, R109A, R136A, E161P, E218A, S215P, 1379A, and M447V, or any combination thereof.

In some embodiments, the recombinant polypeptide is or comprises the sequence set forth in SEQ ID NO: 15. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 15, including a sequence comprising substitution, deletion, and/or insertion at one or more amino acid positions, such as 102, 106, 107, 108, 109, 131, 132, 133, 134, 135, 136, 137, 138, 139, 140, 141, 142, 143, 144, 145, 146, 147, 148, 149, 150, 151, 152, 153, 154, 155, 161, 215, 218, 379, or 447 (amino acid positions with respect to SEQ ID NO: 31 or 32), or any combination thereof. In some embodiments, the recombinant polypeptide is or comprises a variant of SEQ ID NO: 15 and the variant comprises any one, two, three, four, five or more of the mutations selected from the group consisting of P102A, R109A, R136A, E161P, E218A, S215P, 1379A, and M447V, or any combination thereof.

In some embodiments, the recombinant polypeptide is or comprises the sequence set forth in SEQ ID NO: 16. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 16, including a sequence comprising substitution, deletion, and/or insertion at one or more amino acid positions, such as 102, 106, 107, 108, 109, 131, 132, 133, 134, 135, 136, 137, 138, 139, 140, 141, 142, 143, 144, 145, 146, 147, 148, 149, 150, 151, 152, 153, 154, 155, 161, 215, 218, 379, or 447 (amino acid positions with respect to SEQ ID NO: 31 or 32), or any combination thereof. In some embodiments, the recombinant polypeptide is or comprises a variant of SEQ ID NO: 16 and the variant comprises any one, two, three, four, five or more of the mutations selected from the group consisting of P102A, R109A, R136A, E161P, E218A, S215P, 1379A, and M447V, or any combination thereof.

As indicated above, in some embodiments, the recombinant polypeptides provided herein associate not only to form trimers, but can also aggregate or be aggregated to generate proteins comprising a plurality of recombinant polypeptides. In some embodiments, the proteins formed have macrostructures. In some cases, the macrostructure may confer structural stability of the RSV viral antigen or immunogen recombinant polypeptides, which in turn can afford access to potentially antigenic sites capable of promoting an immune response.

In some embodiments, the trimerized recombinant polypeptides aggregate to form a protein containing a plurality of trimerized recombinant polypeptides. In some embodiments, the plurality of trimerized recombinant polypeptides forms a protein having a macrostructure. In some embodiments, the protein comprises a rosette-like oligomer comprising F protein peptide trimers as crutch-shaped rods.

In some embodiments, provided herein is a complex comprising a recombinant polypeptide selected from the group consisting of SEQ ID NOs: 1-16 or a fragment, variant, or mutant thereof, in any suitable combination. In some embodiments, provided herein is a complex comprising a trimer of a recombinant polypeptide selected from the group consisting of SEQ ID NOs: 1-16 or a fragment, variant, or mutant thereof, wherein the recombinant polypeptides are trimerized via inter-polypeptide disulfide bonds to form the trimer.

In some embodiments, the proteins described herein comprising a plurality of recombinant polypeptides are an immunogen. In some embodiments, the proteins described herein comprising a plurality of recombinant polypeptides are comprised in a nanoparticle. For example, in some embodiments, the proteins are linked directly to a nanoparticle, e.g., protein nanoparticle. In some embodiments, the proteins are linked indirectly to a nanoparticle. In some embodiments, the proteins described herein comprising a plurality of recombinant polypeptides are comprised in virus-like particle (VLP).

### 2. Polynucleotides and Vectors

Also provided are polynucleotides (nucleic acid molecules) encoding the RSV antigens or immunogens and recombinant polypeptides provided herein, and vectors for genetically engineering cells to express such RSV antigens or immunogens and recombinant polypeptides.

In some embodiments, provided are polynucleotides that encode recombinant polypeptides provided herein. In some aspects, the polynucleotide contains a single nucleic acid sequence, such as a nucleic acid sequence encoding a recombinant polypeptide. In other instances, the polynucleotide contains a first nucleic acid sequence encoding a recombinant polypeptide a particular RSV viral antigen or immunogen and a second nucleic acid sequence encoding a recombinant polypeptide comprising a different RSV viral antigen or immunogen.

In some embodiments, the polynucleotide encoding the recombinant polypeptide contains at least one promoter that is operatively linked to control expression of the recombinant polypeptide. In some embodiments, the polynucleotide contains two, three, or more promoters operatively linked to control expression of the recombinant polypeptide.

In some embodiments, for example when the polynucleotide contains two or more nucleic acid coding sequences, such as a sequences encoding recombinant polypeptides comprising different RSV viral antigens or immunogens, at least one promoter is operatively linked to control expression of the two or more nucleic acid sequences. In some embodiments, the polynucleotide contains two, three, or more promoters operatively linked to control expression of the recombinant polypeptides.

In some embodiments, expression of the recombinant polypeptide(s) is inducible or conditional. Thus, in some aspects, the polynucleotide encoding the recombinant polypeptide(s) contains a conditional promoter, enhancer, or transactivator. In some such aspects, the conditional promoter, enhancer, or transactivator is an inducible promoter, enhancer, or transactivator or a repressible promoter, enhancer, or transactivator. For example, in some embodiments, an inducible or conditional promoter can be used to restrict expression of the recombinant polypeptides to a specific microenvironment. In some embodiments, expression driven by the inducible or conditional promoter is regulated by exposure to an exogenous agent, such as heat, radiation, or drug.

In cases where the polynucleotide contains more than one nucleic acid sequence encoding a recombinant polypeptide, the polynucleotide may further include a nucleic acid sequence encoding a peptide between the one or more nucleic acid sequences. In some cases, the nucleic acid positioned between the nucleic acid sequences encodes a peptide that separates the translation products of the nucleic acid sequences during or after translation. In some embodiments, the peptide contains an internal ribosome entry site (IRES), a self-cleaving peptide, or a peptide that causes ribosome skipping, such as a T2A peptide.

In some embodiments, the polynucleotide encoding the recombinant polypeptide(s) is introduced into a composition containing cultured cells (e.g., host cells), such as by retroviral transduction, transfection, or transformation. In some embodiments, this can allow for expression (e.g., production) of the recombinant polypeptides. In some embodiments, the expressed recombinant polypeptides are purified.

In some embodiments, the polynucleotide (nucleic acid molecule) provided herein encodes an RSV viral antigen or immunogen as described herein. In some embodiments, the polynucleotide (nucleic acid molecule) provided herein encodes a recombinant polypeptide comprising RSV viral antigen or immunogen, e.g., RSV F peptide protein, as described herein.

Also provided are vectors or constructs containing nucleic acid molecules as described herein. In some embodiments, the vectors or constructs contain one or more promoters operatively linked to the nucleic acid molecule encoding the recombinant polypeptide to drive expression thereof. In some embodiments, the promoter is operatively linked to one or more than one nucleic acid molecule, e.g., nucleic acid molecule encoding recombinant polypeptides containing different RSV viral antigens or immunogens.

In some embodiments, the vector is a viral vector. In some embodiments the viral vector is a retroviral vector. In some embodiments, the retroviral vector is a lentiviral vector. In some embodiments, the retroviral vector is a gammaretroviral vector.

In some embodiments, the vector or construct includes a single promoter that drives the expression of one or more nucleic acid molecules of the polynucleotide. In some embodiments, such promoters can be multicistronic (bicistronic or tricistronic, see e.g., U.S. Pat. No. 6,060,273). For example, in some embodiments, transcription units can be engineered as a bicistronic unit containing an IRES (internal ribosome entry site), which allows coexpression of gene products (e.g., encoding different recombinant polypeptides) by a message from a single promoter. In some embodiments, the vectors provided herein are bicistronic, allowing the vector to contain and express two nucleic acid sequences. In some embodiments, the vectors provided herein are tricistronic, allowing the vector to contain and express three nucleic acid sequences.

In some embodiments, a single promoter directs expression of an RNA that contains, in a single open reading frame (ORF), two or three genes (e.g. encoding the chimeric signaling receptor and encoding a recombinant receptor) separated from one another by sequences encoding a self-cleavage peptide (e.g., 2A sequences) or a protease recognition site (e.g., furin). The ORF thus encodes a single polypeptide, which, either during (in the case of 2A) or after translation, is processed into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Generic Vaccines and Ther. 2:13 (2004) and deFelipe et al. Traffic 5:616-626 (2004)). Many 2A elements are known in the art. Examples of 2A sequences that can be used in the methods and nucleic acids disclosed herein include, without limitation, 2A sequences from the foot-and-mouth disease virus (F2A), equine rhinitis A virus (E2A), Thosea asigna virus (T2A), and porcine teschovirus-1 (P2A) as described in U.S. Patent Publication No. 20070116690.

In some embodiments, the vector is comprised in a virus. In some embodiments, the virus is a pseudovirus. In some embodiments, the virus is a viral-like particle. In some embodiments, the vector is comprised in a cell. In some embodiments, the virus or cell in which the vector is comprised contains a recombinant genome.

### III. Immunogenic Compositions and Formulations

In some embodiments, provided herein is an immunogenic composition comprising a trimer of a recombinant polypeptide comprising a sequence selected from the group consisting of SEQ ID NOs: 1-16, or a combination of any two or more of the trimers. In some embodiments, a unit dose of the immunogenic composition may comprise from about 10 μg to about 100 μg of the RSV F antigen, preferably from about 25 μg to about 75 μg of the RSV F antigen, preferably from about 40 μg to about 60 μg of the RSV F antigen, or about 50 μg of the RSV F antigen. In some embodiments, the dose contains 3 μg of the RSV F antigen. In other embodiments, the dose contains 9 μg of the RSV F antigen. In further embodiments, the dose contains 30 μg of the RSV F antigen.

In some instances it may be desirable to combine a disclosed immunogen, with other pharmaceutical products (e.g., vaccines) which induce protective responses to other agents. For example, a composition including a recombinant RSV F antigen as described herein, e.g., trimer or protein, can be can be administered simultaneously (typically separately) or sequentially with other vaccines recommended by the Advisory Committee on Immunization Practices (ACIP; cdc.gov/vaccines/acip/index.html) for the targeted age group (e.g., infants from approximately one to six months of age), such as an influenza vaccine or a varicella zoster vaccine. As such, a disclosed immunogen including a recombinant RSV F antigen described herein may be administered simultaneously or sequentially with vaccines against, for example, hepatitis B (HepB), diphtheria, tetanus and pertussis (DTaP), pneumococcal bacteria (PCV), Haemopihus influenzae type b (Hib), polio, influenza and rotavirus.

Multivalent or combination vaccines provide protection against multiple pathogens. In some aspects, multivalent vaccines can protect against multiple strains of the same pathogen. In some aspects, multivalent vaccines protect against multiple pathogens, such as the combination vaccine Tdap, which protects against strains of tentus, pertussis, and diphtheria. Multivalent vaccines are highly desirable to minimize the number of immunizations required to confer protection against multiple pathogens or pathogenic strains, to reduce administration costs, and to increase coverage rates. This can be particularly useful, for example, when vaccinating babies or children.

In some embodiments, the vaccine, e.g., comprising an immunogenic composition described herein, is a multivalent vaccine. In some embodiments, the antigenic material for incorporation into the multivalent vaccine compositions of the invention is derived from RSV type A or type B, or a combination thereof. Antigens for incorporation into the multivalent vaccine compositions of the invention may be derived from one strain of RSV or multiple strains, for example, between two and five strains, in order to provide a broader spectrum of protection. In one embodiment, antigens for incorporation into the multivalent vaccine compositions of the invention are derived from multiple strains of RSV virus. Other useful antigens include live, attenuated and inactivated viruses such as inactivated polio virus (Jiang et al., J. Biol. Stand., (1986) 14:103-9), attenuated strains of Hepatitis A virus (Bradley et al., J. Med. Virol., (1984) 14:373-86), attenuated measles virus (James et al., N. Engl. J. Med., (1995) 332:1262-6), and epitopes of pertussis virus (for example, ACEL-IMUNErM acellular DTP, Wyeth-Lederle Vaccines and Pediatrics).

In some aspects, the vaccine provided herein is a universal vaccine. In some embodiments, a universal vaccine is a vaccine which protects against multiple strains of the same virus, such as multiple strains of RSV. Development of an effective universal RSV vaccine would reduce cost and labor, e.g., with seasonal vaccine formulation, and allow for more robust pandemic preparedness.

In some aspects, a universal vaccine is one comprised of multiple epitopes derived from distinct viral strains. In some aspects, a universal vaccine is comprised of a single epitope that is conserved across distinct viral strains. For example, a universal vaccine can be based on the relatively conserved domain(s) of the RSV F protein.

Immunogenic compositions comprising a disclosed immunogen (e.g., a disclosed recombinant RSV F antigen or nucleic acid molecule encoding a protomer of disclosed recombinant RSV F antigen) and a pharmaceutically acceptable carrier are also provided. In some embodiments, the immunogenic composition comprises trimerized recombinant polypeptides provided herein, and optionally a pharmaceutically acceptable carrier. In some embodiments, the immunogenic composition comprises a protein comprising a plurality of trimerized recombinant polypeptides provided herein, and optionally a pharmaceutically acceptable carrier. In some embodiments, the immunogenic composition a protein nanoparticle provided herein, and optionally a pharmaceutically acceptable carrier. In some embodiments, the immunogenic composition comprises a VLP as provided herein, and optionally a pharmaceutically acceptable carrier. In some embodiments, the immunogenic composition comprises an isolated nucleic acid provided herein, and optionally a pharmaceutically acceptable carrier. In some embodiments, the immunogenic composition comprises a vector as provided herein, and optionally a pharmaceutically acceptable carrier. In some embodiments, the immunogenic composition comprises a virus as provided herein, and optionally a pharmaceutically acceptable carrier. In some embodiments, the immunogenic composition comprises a pseudovirus provided herein, and optionally a pharmaceutically acceptable carrier. In some embodiments, the immunogenic composition comprises a cell as provided herein, and optionally a pharmaceutically acceptable carrier. In some embodiments, the immunogenic composition, such as described herein, is a vaccine. In some embodiments, the vaccine is a prophylactic vaccine. In some embodiments, the vaccine is a therapeutic vaccine. In some embodiments, the vaccine is a prophylactic vaccine and a therapeutic vaccine. Such pharmaceutical compositions can be administered to subjects by a variety of administration modes known to the person of ordinary skill in the art, for example, intramuscular, intradermal, subcutaneous, intravenous, intra-arterial, intra-articular, intraperitoneal, intranasal, sublingual, tonsillar, oropharyngeal, or other parenteral and mucosal routes. In several embodiments, pharmaceutical compositions including one or more of the disclosed immunogens are immunogenic compositions. Actual methods for preparing administrable compositions will be known or apparent to those skilled in the art and are described in more detail in such publications as Remingtons Pharmaceutical Sciences, 19th Ed., Mack Publishing Company, Easton, Pa., 1995.

Thus, an immunogen, e.g., recombinant RSV F antigen, e.g., trimer, protein, described herein can be formulated with pharmaceutically acceptable carriers to help retain biological activity while also promoting increased stability during storage within an acceptable temperature range. Potential carriers include, but are not limited to, physiologically balanced culture medium, phosphate buffer saline solution, water, emulsions (e.g., oil/water or water/oil emulsions), various types of wetting agents, cryoprotective additives or stabilizers such as proteins, peptides or hydrolysates (e.g., albumin, gelatin), sugars (e.g., sucrose, lactose, sorbitol), amino acids (e.g., sodium glutamate), or other protective agents. The resulting aqueous solutions may be packaged for use as is or lyophilized. Lyophilized preparations are combined with a sterile solution prior to administration for either single or multiple dosing.

Formulated compositions, especially liquid formulations, may contain a bacteriostat to prevent or minimize degradation during storage, including but not limited to effective concentrations (usually 1% w/v) of benzyl alcohol, phenol, m-cresol, chlorobutanol, methylparaben, and/or propylparaben. A bacteriostat may be contraindicated for some patients; therefore, a lyophilized formulation may be reconstituted in a solution either containing or not containing such a component.

The immunogenic compositions of the disclosure can contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate. The immunogenic composition may optionally include an adjuvant to enhance an immune response of the host. Suitable adjuvants are, for example, toll-like receptor agonists, alum, AlPO₄, alhydrogel, Lipid-A and derivatives or variants thereof, oil-emulsions, saponins, neutral liposomes, liposomes containing the vaccine and cytokines, non-ionic block copolymers, and chemokines. Non-ionic block polymers containing polyoxyethylene (POE) and polyxylpropylene (POP), such as POE-POP-POE block copolymers, MPL™ (3-O-deacylated monophosphoryl lipid A; Corixa, Hamilton, Ind.) and IL-12 (Genetics Institute, Cambridge, Mass.), among many other suitable adjuvants well known in the art, may be used as an adjuvant (Newman et al., 1998, Critical Reviews in Therapeutic Drug Carrier Systems 15:89-142). These adjuvants have the advantage in that they help to stimulate the immune system in a non-specific way, thus enhancing the immune response to a pharmaceutical product. In some embodiments, the immunogenic compositions of the disclosure may include or be administered with more than one adjuvant. In some embodiments, the immunogenic compositions of the disclosure may include or be administered with two adjuvants. In some embodiments, the immunogenic compositions of the disclosure may include or be administered with a plurality of adjuvants. For example, in some cases, a vaccine, e.g., comprising an immunogenic composition provided herein, may include or be administered in combination with a plurality of adjuvants.

For vaccine compositions, examples of suitable adjuvants include, e.g., aluminum hydroxide, lecithin, Freund's adjuvant, MPL™ and IL-12. In some embodiments, the vaccine compositions or nanoparticle immunogens disclosed herein (e.g., RSV vaccine composition) can be formulated as a controlled-release or time-release formulation. This can be achieved in a composition that contains a slow release polymer or via a microencapsulated delivery system or bioadhesive gel. The various pharmaceutical compositions can be prepared in accordance with standard procedures well known in the art.

In some embodiments, the immunogenic compositions of the disclosure can contain an adjuvant formulation comprising a metabolizable oil (e.g., squalene) and alpha tocopherol in the form of an oil-in-water emulsion, and polyoxyethylene sorbitan monooleate (Tween-80). In some embodiments, the adjuvant formulation can comprise from about 2% to about 10% squalene, from about 2 to about 10% alpha tocopherol (e.g., D-alpha-tocopherol) and from about 0.3 to about 3% polyoxyethylene sorbitan monooleate. In some embodiments, the adjuvant formulation can comprise about 5% squalene, about 5% tocopherol, and about 0.4% polyoxyethylene sorbitan monooleate. In some embodiments, the immunogenic compositions of the disclosure can contain 3 de-O-acylated monophosphoryl lipid A (3D-MPL), and an adjuvant in the form of an oil in water emulsion, which adjuvant contains a metabolizable oil, alpha tocopherol, and polyoxyethylene sorbitan monoleate. In some embodiments, the immunogenic compositions of the disclosure can contain QS21 (extract of Quillaja saponaria Molina: fraction 21), 3D-MPL and an oil in water emulsion wherein the oil in water emulsion comprises a metabolizable oil, alpha tocopherol and polyoxyethelene sorbitan monooleate. In some embodiments, the immunogenic compositions of the disclosure can contain QS21, 3D-MPL and an oil in water emulsion wherein the oil in water emulsion has the following composition: a metabolisible oil, such as squalene, alpha tocopherol and Tween-80. In some embodiments, the immunogenic compositions of the disclosure can contain an adjuvant in the form of a liposome composition.

In some embodiments, the immunogenic compositions of the disclosure can contain an adjuvant formulation comprising a metabolizable oil (e.g., squalene), polyoxyethylene sorbitan monooleate (Tween-80), and Span 85. In some embodiments, the adjuvant formulation can comprise about 5% (w/v) squalene, about 0.5% (w/v) polyoxyethylene sorbitan monooleate, and about about 0.5% (w/v) Span 85.

In some embodiments, the immunogenic compositions of the disclosure can contain an adjuvant formulation comprising Quillaja saponins, cholesterol, and phosphorlipid, e.g., in the form of a nanoparticle composition. In some embodiments, the immunogenic compositions of the disclosure can contain a mixture of separately purified fractions of Quillaja saponaria Molina where are subsequently formulated with cholesterol and phospholipid.

In some embodiments, the immunogenic compositions of the disclosure can contain an adjuvant selected from the group consisting of MF59™, Matrix-A™, Matrix-C™, Matrix-M™, AS01, AS02, AS03, and AS04.

In some embodiments, the immunogenic compositions of the disclosure can contain a toll-like receptor 9 (TLR9) agonist, wherein the TLR9 agonist is an oligonucleotide of from 8 to 35 nucleotides in length comprising an unmethylated cytidine-phospho-guanosine (also referred to as CpG or cytosine-phosphate-guanosine) motif, and the RSV antigen and the oligonucleotide are present in the immunogenic composition in amounts effective to stimulate an immune response against the RSV antigen in a mammalian subject, such as a human subject in need thereof. TLR9 (CD289) recognizes unmethylated cytidine-phospho-guanosine (CpG) motifs found in microbial DNA, which can be mimicked using synthetic CpG-containing oligodeoxynucleotides (CpG-ODNs). CpG-ODNs are known to enhance antibody production and to stimulate T helper 1 (Th1) cell responses (Coffman et al., Immunity, 33:492-503, 2010). Optimal oligonucleotide TLR9 agonists often contain a palindromic sequence following the general formula of; 5′-purine-purine-CG-pyrimidine-pyrimidine-3′, or 5′-purine-purine-CG-pyrimidine-pyrimidine-CG-3′. U.S. Pat. No. 6,589,940, which is incorporated herein by reference in its entirety. In some embodiments, the CpG oligonucleotide is linear. In other embodiments, the CpG oligonucleotide is circular or includes hairpin loop(s). The CpG oligonucleotide may be single stranded or double stranded. In some embodiments, the CpG oligonucleotide may contain modifications. Modifications include but are not limited to, modifications of the 3′OH or 5′OH group, modifications of the nucleotide base, modifications of the sugar component, and modifications of the phosphate group. Modified bases may be included in the palindromic sequence of the CpG oligonucleotide as long as the modified base(s) maintains the same specificity for its natural complement through Watson-Crick base pairing (e.g., the palindromic portion is still self-complementary). In some embodiments, the CpG oligonucleotide comprises a non-canonical base. In some embodiments, the CpG oligonucleotide comprises a modified nucleoside. In some embodiments, the modified nucleoside is selected from the group consisting of 2′-deoxy-7-deazaguanosine, 2′-deoxy-6-thioguanosine, arabinoguanosine, 2′-deoxy-2′substituted-arabinoguanosine, and 2′-O-substituted-arabinoguanosine. The CpG oligonucleotide may contain a modification of the phosphate group. For example, in addition to phosphodiester linkages, phosphate modifications include, but are not limited to, methyl phosphonate, phosphorothioate, phosphoramidate (bridging or non-bridging), phosphotriester and phosphorodithioate and may be used in any combination. Other non-phosphate linkages may also be used. In some embodiments, the oligonucleotides comprise only phosphorothioate backbones. In some embodiments, the oligonucleotides comprise only phosphodiester backbones. In some embodiments, the oligonucleotide comprises a combination of phosphate linkages in the phosphate backbone such as a combination of phosphodiester and phosphorothioate linkages. Oligonucleotides with phosphorothioate backbones can be more immunogenic than those with phosphodiester backbones and appear to be more resistant to degradation after injection into the host (Braun et al., J Immunol, 141:2084-2089, 1988; and Latimer et al., Mol Immunol, 32:1057-1064, 1995). The CpG oligonucleotides of the present disclosure include at least one, two or three internucleotide phosphorothioate ester linkages. In some embodiments, when a plurality of CpG oligonucleotide molecules are present in a pharmaceutical composition comprising at least one excipient, both stereoisomers of the phosphorothioate ester linkage are present in the plurality of CpG oligonucleotide molecules. In some embodiments, all of the internucleotide linkages of the CpG oligonucleotide are phosphorothioate linkages, or said another way, the CpG oligonucleotide has a phosphorothioate backbone.

Any suitable CpG oligodeoxynucleotides (ODNs) or combinations thereof can be used as adjuvants in the present disclosure. For instance, K-type ODNs (also referred to as B type) encode multiple CpG motifs on a phosphorothioate backbone. K-type ODNs may be based on the following sequence TCCATGGACGTTCCTGAGCGTT. The use of phosphorothioate nucleotides enhances resistance to nuclease digestion when compared with native phosphodiester nucleotides, resulting in a substantially longer in vivo half life. K-type ODNs trigger pDCs to differentiate and produce TNF-α, and B cells to proliferate and secrete IgM. D-type ODNs (also referred to as A type) are constructed of a mixed phosphodiester/phosphorothioate backbone, contain a single CpG motif flanked by palindromic sequences and have poly G tails at the 3′ and 5′ ends (a structural motif that facilitates the formation of concatamers). D-type ODNs may be based on the following sequence GGTGCATCGATGCAGGGGGG. D-type ODNs trigger pDCs to mature and secrete IFN-α, but have no effect on B cells. C-type ODNs resemble K-type in being composed entirely of phosphorothioate nucleotides, but resemble D-type in containing palindromic CpG motifs. C-type ODNs may be based on the following sequence TCGTCGTTCGAACGACGTTGAT. This class of ODNs stimulate B cells to secrete IL-6 and pDCs to produce IFN-α. P-type ODNs contain two palindromic sequences, enabling them to form higher ordered structures. P-type ODNs may be based on the following sequence TCGTCGACGATCGGCGCGCGCCG. P-type ODNs activate B cells and pDCs, and induce substantially greater IFN-α production when compared with C-type ODNs. In this paragraph, bold letters in ODN sequences indicate self-complementary palindromes and CpG motifs are underlined.

Exemplary CpG ODNs, e.g., CpG 7909 (5′-TCGTCGTTTTGTCGTTTTGTCGTT-3′) and CpG 1018 (5′-TGACTGTGAACGTTCGAGATGA-3′), are known and disclosed in U.S. Pat. Nos. 7,255,868, 7,491,706, 7,479,285, 7,745,598, 7,785,610, 8,003,115, 8,133,874, 8,114,418, 8,222,398, 8,333,980, 8,597,665, 8,669,237, 9,028,845, and 10,052,378; application publication US 2020/0002704; and Bode et al., “CpG DNA as a vaccine adjuvant”, Expert Rev Vaccines (2011), 10(4): 499-511, all of which are incorporated herein by reference in their entireties for all purposes.

One or more adjuvants may be used in combination and may include, but are not limited to, alum (aluminum salts), oil-in-water emulsions, water-in-oil emulsions, liposomes, and microparticles, such as poly(lactide-co-glycolide) microparticles (Shah et al., Methods Mol Biol, 1494:1-14, 2017). In some embodiments, the immunogenic compositions further comprises an aluminum salt adjuvant to which the RSV antigen is adsorbed. In some embodiments, the aluminum salt adjuvant comprises one or more of the group consisting of amorphous aluminum hydroxyphosphate sulfate, aluminum hydroxide, aluminum phosphate, and potassium aluminum sulfate. In some embodiments, the aluminum salt adjuvant comprises one or both of aluminum hydroxide and aluminum phosphate. In some embodiments, the aluminum salt adjuvant comprises aluminum hydroxide. In some embodiments, a unit dose of the immunogenic composition comprises from about 0.25 to about 0.50 mg Al³⁺, or about 0.35 mg Al³⁺. In some embodiments, the immunogenic composition further comprises an additional adjuvant. Other suitable adjuvants include, but are not limited to, squalene-in-water emulsion (e.g., MF59 or AS03), TLR3 agonists (e.g., poly-IC or poly-ICLC), TLR4 agonists (e.g., bacterial lipopolysaccharide derivatives such monophosphoryl lipid A (MPL), and/or a saponin such as Quil A or QS-21, as in AS01 or AS02), a TLR5 agonist (bacterial flagellin), and TLR7, TLR8 and/or TLR9 agonists (imidazoquinoline derivatives such as imiquimod, and resiquimod) (Coffman et al., Immunity, 33:492-503, 2010). In some embodiments, the additional adjuvant comprises MPL and alum (e.g., AS04). For veterinary use and for production of antibodies in non-human animals, mitogenic components of Freund's adjuvant (both complete and incomplete) can be used.

In some embodiments, the immunogenic compositions comprise pharmaceutically acceptable excipients including for instance, solvents, bulking agents, buffering agents, tonicity adjusting agents, and preservatives (Pramanick et al., Pharma Times, 45:65-77, 2013). In some embodiments the immunogenic compositions may comprise an excipient that functions as one or more of a solvent, a bulking agent, a buffering agent, and a tonicity adjusting agent (e.g., sodium chloride in saline may serve as both an aqueous vehicle and a tonicity adjusting agent).

In some embodiments, the immunogenic compositions comprise an aqueous vehicle as a solvent. Suitable vehicles include for instance sterile water, saline solution, phosphate buffered saline, and Ringer's solution. In some embodiments, the composition is isotonic.

The immunogenic compositions may comprise a buffering agent. Buffering agents control pH to inhibit degradation of the active agent during processing, storage and optionally reconstitution. Suitable buffers include for instance salts comprising acetate, citrate, phosphate or sulfate. Other suitable buffers include for instance amino acids such as arginine, glycine, histidine, and lysine. The buffering agent may further comprise hydrochloric acid or sodium hydroxide. In some embodiments, the buffering agent maintains the pH of the composition within a range of 6 to 9. In some embodiments, the pH is greater than (lower limit) 6, 7 or 8. In some embodiments, the pH is less than (upper limit) 9, 8, or 7. That is, the pH is in the range of from about 6 to 9 in which the lower limit is less than the upper limit.

The immunogenic compositions may comprise a tonicity adjusting agent. Suitable tonicity adjusting agents include for instance dextrose, glycerol, sodium chloride, glycerin and mannitol.

The immunogenic compositions may comprise a bulking agent. Bulking agents are particularly useful when the pharmaceutical composition is to be lyophilized before administration. In some embodiments, the bulking agent is a protectant that aids in the stabilization and prevention of degradation of the active agents during freeze or spray drying and/or during storage. Suitable bulking agents are sugars (mono-, di- and polysaccharides) such as sucrose, lactose, trehalose, mannitol, sorbital, glucose and raffinose.

The immunogenic compositions may comprise a preservative. Suitable preservatives include for instance antioxidants and antimicrobial agents. However, in preferred embodiments, the immunogenic composition is prepared under sterile conditions and is in a single use container, and thus does not necessitate inclusion of a preservative.

In some embodiments, the composition can be provided as a sterile composition. The pharmaceutical composition typically contains an effective amount of a disclosed immunogen and can be prepared by conventional techniques. Typically, the amount of immunogen in each dose of the immunogenic composition is selected as an amount which induces an immune response without significant, adverse side effects. In some embodiments, the composition can be provided in unit dosage form for use to induce an immune response in a subject. A unit dosage form contains a suitable single preselected dosage for administration to a subject, or suitable marked or measured multiples of two or more preselected unit dosages, and/or a metering mechanism for administering the unit dose or multiples thereof. In other embodiments, the composition further includes an adjuvant.

### IV. Methods of Inducing an Immune Response

In some embodiments, provided herein is a method for generating an immune response to a surface antigen of RSV in a subject, comprising administering to the subject an effective amount of a complex comprising a recombinant polypeptide selected from the group consisting of SEQ ID NOs: 1-16. In some embodiments, provided herein is a method for generating an immune response to a surface antigen of RSV in a subject, wherein the surface antigen comprises an F protein or antigenic fragment thereof, and the method comprises administering to the subject an effective amount of a complex comprising a recombinant polypeptide selected from the group consisting of SEQ ID NOs: 1-16. In some embodiments, provided herein is a method for generating an immune response to a surface antigen of RSV in a subject, wherein the surface antigen comprises a sequence selected from the group consisting of SEQ ID NOs: 17-47, and the method comprises administering to the subject an effective amount of a complex comprising a recombinant polypeptide selected from the group consisting of SEQ ID NOs: 1-16. In some embodiments, provided herein is a method for generating an immune response to a surface antigen of RSV in a subject, wherein the surface antigen comprises an F protein or antigenic fragment thereof of RSV and optionally the surface antigen comprises a sequence of any one or more of SEQ ID NOs: 17-47 or antigenic fragment thereof, and the method comprises administering to the subject an effective amount of a complex comprising a recombinant polypeptide comprising the sequence set forth in any one of SEQ ID NOs: 1-16.

In some embodiments, provided herein is a method for generating an immune response to a surface antigen of RSV in a subject, wherein the surface antigen comprises an F protein or antigenic fragment thereof, and the method comprises administering to the subject an effective amount of a complex comprising a recombinant polypeptide comprising the sequence selected from the group consisting of SEQ ID NOs: 1-16, or a combination of any two or more of the complexes.

The disclosed immunogens (e.g., recombinant RSV F antigen, e.g., trimer, protein described herein, a nucleic acid molecule (such as an RNA molecule) or vector encoding a protomer of a disclosed recombinant RSV F antigen, or a protein nanoparticle or virus like particle comprising a disclosed recombinant RSV F antigen) can be administered to a subject to induce an immune response to the corresponding RSV F antigen in the subject. In a particular example, the subject is a human. The immune response can be a protective immune response, for example a response that inhibits subsequent infection with the corresponding RSV. Elicitation of the immune response can also be used to treat or inhibit infection and illnesses associated with the corresponding RSV.

A subject can be selected for treatment that has, or is at risk for developing infection with the RSV, for example because of exposure or the possibility of exposure to the RSV. Following administration of a disclosed immunogen, the subject can be monitored for infection or symptoms associated with RSV, or both.

Typical subjects intended for treatment with the therapeutics and methods of the present disclosure include humans, as well as non-human primates and other animals. To identify subjects for prophylaxis or treatment according to the methods of the disclosure, accepted screening methods are employed to determine risk factors associated with a targeted or suspected disease or condition, or to determine the status of an existing disease or condition in a subject. These screening methods include, for example, conventional work-ups to determine environmental, familial, occupational, and other such risk factors that may be associated with the targeted or suspected disease or condition, as well as diagnostic methods, such as various ELISA and other immunoassay methods to detect and/or characterize RSV infection. These and other routine methods allow the clinician to select patients in need of therapy using the methods and pharmaceutical compositions of the disclosure. In accordance with these methods and principles, a composition can be administered according to the teachings herein, or other conventional methods, as an independent prophylaxis or treatment program, or as a follow-up, adjunct or coordinate treatment regimen to other treatments.

The administration of a disclosed immunogen, e.g., RSV F antigen, e.g., trimer, protein, can be for prophylactic or therapeutic purpose. When provided prophylactically, the disclosed therapeutic agents are provided in advance of any symptom, for example, in advance of infection. The prophylactic administration of the disclosed therapeutic agents serves to prevent or ameliorate any subsequent infection. When provided therapeutically, the disclosed therapeutic agents are provided at or after the onset of a symptom of disease or infection, for example, after development of a symptom of infection with RSV corresponding to the RSV F antigen, or after diagnosis with the RSV infection. The therapeutic agents can thus be provided prior to the anticipated exposure to RSV so as to attenuate the anticipated severity, duration or extent of an infection and/or associated disease symptoms, after exposure or suspected exposure to the virus, or after the actual initiation of an infection.

The immunogens described herein, and immunogenic compositions thereof, are provided to a subject in an amount effective to induce or enhance an immune response against the RSV F antigen in the subject, preferably a human. The actual dosage of disclosed immunogen will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the composition for eliciting the desired activity or biological response in the subject. Dosage regimens can be adjusted to provide an optimum prophylactic or therapeutic response.

An immunogenic composition including one or more of the disclosed immunogens can be used in coordinate (or prime-boost) vaccination protocols or combinatorial formulations. In certain embodiments, novel combinatorial immunogenic compositions and coordinate immunization protocols employ separate immunogens or formulations, each directed toward eliciting an anti-viral immune response, such as an immune response to RSV F antigen. Separate immunogenic compositions that elicit the anti-viral immune response can be combined in a polyvalent immunogenic composition administered to a subject in a single immunization step, or they can be administered separately (in monovalent immunogenic compositions) in a coordinate (or prime-boost) immunization protocol.

There can be several boosts, and each boost can be a different disclosed immunogen. In some examples that the boost may be the same immunogen as another boost, or the prime. The prime and boost can be administered as a single dose or multiple doses, for example two doses, three doses, four doses, five doses, six doses or more can be administered to a subject over days, weeks or months. Multiple boosts can also be given, such one to five (e.g., 1, 2, 3, 4 or 5 boosts), or more. Different dosages can be used in a series of sequential immunizations. For example a relatively large dose in a primary immunization and then a boost with relatively smaller doses.

In some embodiments, the boost can be administered about two, about three to eight, or about four, weeks following the prime, or about several months after the prime. In some embodiments, the boost can be administered about 5, about 6, about 7, about 8, about 10, about 12, about 18, about 24, months after the prime, or more or less time after the prime. Periodic additional boosts can also be used at appropriate time points to enhance the subject's “immune memory.” The adequacy of the vaccination parameters chosen, e.g., formulation, dose, regimen and the like, can be determined by taking aliquots of serum from the subject and assaying antibody titers during the course of the immunization program. In addition, the clinical condition of the subject can be monitored for the desired effect, e.g., prevention of infection or improvement in disease state (e.g., reduction in viral load). If such monitoring indicates that vaccination is sub-optimal, the subject can be boosted with an additional dose of immunogenic composition, and the vaccination parameters can be modified in a fashion expected to potentiate the immune response.

In some embodiments, the prime-boost method can include DNA-primer and protein-boost vaccination protocol to a subject. The method can include two or more administrations of the nucleic acid molecule or the protein.

For protein therapeutics, typically, each human dose will comprise 1-1000 μg of protein, such as from about 1 μg to about 100 μg, for example, from about 1 μg to about 50 μg, such as about 1 μg, about 2 μg, about 5 μg, about 10 μg, about 15 μg, about 20 μg, about 25 μg, about 30 μg, about 40 μg, or about 50 μg.

The amount utilized in an immunogenic composition is selected based on the subject population (e.g., infant or elderly). An optimal amount for a particular composition can be ascertained by standard studies involving observation of antibody titers and other responses in subjects. It is understood that a therapeutically effective amount of a disclosed immunogen, such as a disclosed recombinant RSV F antigen, e.g., trimer, protein, viral vector, or nucleic acid molecule in a immunogenic composition, can include an amount that is ineffective at eliciting an immune response by administration of a single dose, but that is effective upon administration of multiple dosages, for example in a prime-boost administration protocol.

Upon administration of a disclosed immunogen of this disclosure, the immune system of the subject typically responds to the immunogenic composition by producing antibodies specific for the RSV F protein peptide included in the immunogen. Such a response signifies that an immunologically effective dose was delivered to the subject.

In some embodiments, the antibody response of a subject will be determined in the context of evaluating effective dosages/immunization protocols. In most instances it will be sufficient to assess the antibody titer in serum or plasma obtained from the subject. Decisions as to whether to administer booster inoculations and/or to change the amount of the therapeutic agent administered to the individual can be at least partially based on the antibody titer level. The antibody titer level can be based on, for example, an immunobinding assay which measures the concentration of antibodies in the serum which bind to an antigen including, for example, the recombinant RSV F antigen, e.g., trimer, protein.

RSV infection does not need to be completely eliminated or reduced or prevented for the methods to be effective. For example, elicitation of an immune response to an RSV with one or more of the disclosed immunogens can reduce or inhibit infection with the RSV by a desired amount, for example, by at least 10%, at least 20%, at least 50%, at least 60%, at least 70%, at least 80%, at least 90%, at least 95%, at least 98%, or even at least 100% (elimination or prevention of detectable infected cells), as compared to infection with the RSV in the absence of the immunogen. In additional examples, RSV replication can be reduced or inhibited by the disclosed methods. RSV replication does not need to be completely eliminated for the method to be effective. For example, the immune response elicited using one or more of the disclosed immunogens can reduce replication of the corresponding RSV by a desired amount, for example, by at least 10%, at least 20%, at least 50%, at least 60%, at least 70%, at least 80%, at least 90%, at least 95%, at least 98%, or even at least 100% (elimination or prevention of detectable replication of the RSV), as compared to replication of the RSV in the absence of the immune response.

In some embodiments, the disclosed immunogen is administered to the subject simultaneously with the administration of the adjuvant. In other embodiments, the disclosed immunogen is administered to the subject after the administration of the adjuvant and within a sufficient amount of time to induce the immune response.

One approach to administration of nucleic acids is direct immunization with plasmid DNA, such as with a mammalian expression plasmid. Immunization by nucleic acid constructs is well known in the art and taught, for example, in U.S. Pat. No. 5,643,578 (which describes methods of immunizing vertebrates by introducing DNA encoding a desired antigen to elicit a cell-mediated or a humoral response), and U.S. Pat. Nos. 5,593,972 and 5,817,637 (which describe operably linking a nucleic acid sequence encoding an antigen to regulatory sequences enabling expression). U.S. Pat. No. 5,880,103 describes several methods of delivery of nucleic acids encoding immunogenic peptides or other antigens to an organism. The methods include liposomal delivery of the nucleic acids (or of the synthetic peptides themselves), and immune-stimulating constructs, or ISCOMS™, negatively charged cage-like structures of 30-40 nm in size formed spontaneously on mixing cholesterol and Quil A™ (saponin). Protective immunity has been generated in a variety of experimental models of infection, including toxoplasmosis and Epstein-Barr virus-induced tumors, using ISCOMS™ as the delivery vehicle for antigens (Mowat and Donachie, Immunol. Today 12:383, 1991). Doses of antigen as low as 1 μg encapsulated in ISCOMS™ have been found to produce Class I mediated CTL responses (Takahashi et al., Nature 344:873, 1990).

In some embodiments, a plasmid DNA vaccine is used to express a disclosed immunogen in a subject. For example, a nucleic acid molecule encoding a disclosed immunogen can be administered to a subject to induce an immune response to the RSV F antigen. In some embodiments, the nucleic acid molecule can be included on a plasmid vector for DNA immunization, such as the pVRC8400 vector (described in Barouch et al., J. Virol, 79, 8828-8834, 2005, which is incorporated by reference herein).

In another approach to using nucleic acids for immunization, a disclosed recombinant RSV F antigen, e.g., trimer, protein, can be expressed by attenuated viral hosts or vectors or bacterial vectors. Recombinant vaccinia virus, adeno-associated virus (AAV), herpes virus, retrovirus, cytogmeglo virus or other viral vectors can be used to express the peptide or protein, thereby eliciting a CTL response. For example, vaccinia vectors and methods useful in immunization protocols are described in U.S. Pat. No. 4,722,848. BCG (Bacillus Calmette Guerin) provides another vector for expression of the peptides (see Stover, Nature 351:456-460, 1991).

In one embodiment, a nucleic acid encoding a disclosed recombinant RSV F antigen is introduced directly into cells. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter. Typically, the DNA is injected into muscle, although it can also be injected directly into other sites. Dosages for injection are usually around 0.5 μg/kg to about 50 mg/kg, and typically are about 0.005 mg/kg to about 5 mg/kg (see, e.g., U.S. Pat. No. 5,589,466).

For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter. Typically, the DNA is injected into muscle, although it can also be injected directly into other sites. Dosages for injection are usually around 0.5 μg/kg to about 50 mg/kg, and typically are about 0.005 mg/kg to about 5 mg/kg (see, e.g., U.S. Pat. No. 5,589,466).

In another embodiment, an mRNA-based immunization protocol can be used to deliver a nucleic acid encoding a disclosed recombinant RSV F antigen directly into cells. In some embodiments, nucleic acid-based vaccines based on mRNA may provide a potent alternative to the previously mentioned approaches. mRNA vaccines preclude safety concerns about DNA integration into the host genome and can be directly translated in the host cell cytoplasm. Moreover, the simple cell-free, in vitro synthesis of RNA avoids the manufacturing complications associated with viral vectors. Two exemplary forms of RNA-based vaccination that can be used to deliver a nucleic acid encoding a disclosed recombinant RSV F antigen include conventional non-amplifying mRNA immunization (see, e.g., Petsch et al., “Protective efficacy of in vitro synthesized, specific mRNA vaccines against influenza A virus infection,” Nature biotechnology, 30(12):1210-6, 2012) and self-amplifying mRNA immunization (see, e.g., Geall et al., “Nonviral delivery of self-amplifying RNA vaccines,” PNAS, 109(36): 14604-14609, 2012; Magini et al., “Self-Amplifying mRNA Vaccines Expressing Multiple Conserved Influenza Antigens Confer Protection against Homologous and Heterosubtypic Viral Challenge,” PLoS One, 11(8):e0161193, 2016; and Brito et al., “Self-amplifying mRNA vaccines,” Adv Genet., 89:179-233, 2015).

In some embodiments, administration of a therapeutically effective amount of one or more of the disclosed immunogens to a subject induces a neutralizing immune response in the subject. To assess neutralization activity, following immunization of a subject, serum can be collected from the subject at appropriate time points, frozen, and stored for neutralization testing. Methods to assay for neutralization activity are known to the person of ordinary skill in the art and are further described herein, and include, but are not limited to, plaque reduction neutralization (PRNT) assays, microneutralization assays, flow cytometry based assays, single-cycle infection assays. In some embodiments, the serum neutralization activity can be assayed using a panel of RSV pseudoviruses.

In some embodiments, administration of a therapeutically effective amount of one or more of the disclosed immunogens to a subject induces a neutralizing immune response in the subject. To assess neutralization activity, following immunization of a subject, serum can be collected from the subject at appropriate time points, frozen, and stored for neutralization testing. Methods to assay for neutralization activity are known to the person of ordinary skill in the art and are further described herein, and include, but are not limited to, plaque reduction neutralization (PRNT) assays, microneutralization assays, flow cytometry based assays, single-cycle infection assays. In some embodiments, the serum neutralization activity can be assayed using a panel of RSV pseudoviruses.

In some embodiments, a neutralizing immune response induced by the disclosed immunogens herein generates a neutralizing antibody against RSV. In some embodiments, the neutralizing antibody herein binds to a cellular receptor or coreceptor of RSV or component thereof. Nucleolin is an entry coreceptor for RSV and also mediates the cellular entry of influenza, the parainfluenza virus, some enteroviruses and the bacterium that causes tularaemia. Binding of the prefusion RSV-F glycoprotein with the insulin-like growth factor-1 receptor (IGF1R) may also trigger the activation of protein kinase C zeta (PKCC), recruiting nucleolin from the nuclei of cells to the plasma membrane to bind to RSV-F on virions. In some embodiments, the viral receptor or coreceptor is a paramyxovirus receptor or coreceptor, preferably a pneumonia virus receptor or coreceptor, more preferably a human RSV receptor or coreceptor. For instance, CCR1, CCR2, CCR3, CCR4, CCR5 and/or CCR8 receptors may be involved in human RSV infection. RhoA is another example of host cell RSV receptor or coreceptor. In some embodiments, the neutralizing antibody herein modulates, decreases, antagonizes, mitigates, blocks, inhibits, abrogates and/or interferes with at least one RSV activity or binding, or with RSV receptor activity or binding, in vitro, in situ and/or in vivo, such as RSV release, RSV receptor signaling, membrane RSV cleavage, RSV activity, RSV production and/or synthesis. In some embodiments, the disclosed immunogens herein induce neutralizing antibodies against RSV that modulate, decrease, antagonize, mitigate, block, inhibit, abrogate and/or interfere with RSV binding to a RSV receptor or coreceptor, such as nucleolin, IGF1R, CCR1, CCR2, CCR3, CCR4, CCR5, CCR8 and/or RhoA.

### V. Articles of Manufacture or Kits

Also provided are articles of manufacture or kits containing the provided recombinant polypeptide, proteins, and immunogenic compositions. The articles of manufacture may include a container and a label or package insert on or associated with the container. Suitable containers include, for example, bottles, vials, syringes, test tubes, IV solution bags, etc. The containers may be formed from a variety of materials such as glass or plastic. In some embodiments, the container has a sterile access port. Exemplary containers include an intravenous solution bags, vials, including those with stoppers pierceable by a needle for injection. The article of manufacture or kit may further include a package insert indicating that the compositions can be used to treat a particular condition such as a condition described herein (e.g., RSV infection). Alternatively, or additionally, the article of manufacture or kit may further include another or the same container comprising a pharmaceutically-acceptable buffer. It may further include other materials such as other buffers, diluents, filters, needles, and/or syringes.

The label or package insert may indicate that the composition is used for treating an RSV infection in an individual. The label or a package insert, which is on or associated with the container, may indicate directions for reconstitution and/or use of the formulation. The label or package insert may further indicate that the formulation is useful or intended for subcutaneous, intravenous, or other modes of administration for treating or preventing a RSV infection in an individual.

The container in some embodiments holds a composition which is by itself or combined with another composition effective for treating, preventing and/or diagnosing the condition. The article of manufacture or kit may include (a) a first container with a composition contained therein (i.e., first medicament), wherein the composition includes the immunogenic composition or protein or recombinant polypeptide thereof; and (b) a second container with a composition contained therein (i.e., second medicament), wherein the composition includes a further agent, such as an adjuvant or otherwise therapeutic agent, and which article or kit further comprises instructions on the label or package insert for treating the subject with the second medicament, in an effective amount.

### Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polypeptide” and “protein” are used interchangeably to refer to a polymer of amino acid residues, and are not limited to a minimum length. Polypeptides, including the provided receptors and other polypeptides, e.g., linkers or peptides, may include amino acid residues including natural and/or non-natural amino acid residues. The terms also include post-expression modifications of the polypeptide, for example, glycosylation, sialylation, acetylation, and phosphorylation. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

As used herein, a “subject” is a mammal, such as a human or other animal, and typically is human. In some embodiments, the subject, e.g., patient, to whom the agent or agents, cells, cell populations, or compositions are administered, is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. In some embodiments, sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the cells and/or compositions at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount. In the context of lower tumor burden, the prophylactically effective amount in some aspects will be higher than the therapeutically effective amount.

The term “about” as used herein refers to the usual error range for the respective value readily known to the skilled person in this technical field. Reference to “about” a value or parameter herein includes (and describes) embodiments that are directed to that value or parameter per se.

As used herein, the singular forms “a,” “an,” and “the” include plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

As used herein, a composition refers to any mixture of two or more products, substances, or compounds, including cells. It may be a solution, a suspension, liquid, powder, a paste, aqueous, non-aqueous or any combination thereof.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### EXEMPLARY EMBODIMENTS

Embodiment 1. A protein comprising a plurality of recombinant polypeptides, each recombinant polypeptide comprising a respiratory syncytial virus (RSV) F protein peptide or a fragment or epitope thereof linked to a C-terminal propeptide of collagen, wherein the C-terminal propeptides of the recombinant polypeptides form inter-polypeptide disulfide bonds.

Embodiment 2. The protein of embodiment 1, wherein the RSV is of subtype A or subtype B.

Embodiment 3. The protein of embodiment 1 or 2, wherein the epitope is a linear epitope or a conformational epitope.

Embodiment 4. The protein of any of embodiments 1-3, wherein the F protein peptide comprises an F1 subunit peptide, an F2 subunit peptide, or any combination thereof, and the protein comprises three recombinant polypeptides.

Embodiment 5. The protein of any of embodiments 1-4, wherein the F protein peptide comprises a signal peptide, a heptad-repeat C (HRC) peptide, a pep27 peptide, a fusion peptide (FP), a heptad-repeat A (HRA) peptide, a Domain I peptide, a Domain H peptide, or heptad-repeat B (HRB) peptide, or any combination thereof.

Embodiment 6. The protein of any of embodiments 1-5, wherein the F protein peptide comprises an F1 subunit but not an F2 subunit of the F protein, or vice versa.

Embodiment 7. The protein of any of embodiments 1-6, wherein the F protein peptide comprises an F1 subunit and an F2 subunit of the F protein, optionally without pep27, and optionally wherein the F1 subunit and the F2 subunit are linked by a disulfide bond or an artificially introduced linker.

Embodiment 8. The protein of any of embodiments 1-7, wherein the F protein peptide does not comprise a transmembrane (TM) domain peptide and/or a cytoplasm (CP) domain peptide.

Embodiment 9. The protein of any of embodiments 1-8, wherein the F protein peptide comprises a protease cleavage site, wherein the protease is optionally furin, trypsin, factor Xa, or cathepsin L.

Embodiment 10. The protein of any of embodiments 1-8, wherein the F protein peptide does not comprise a protease cleavage site, wherein the protease is optionally furin, trypsin, factor Xa, or cathepsin L.

Embodiment 11. The protein of any of embodiments 1-10, wherein the F protein peptide is soluble or does not directly bind to a lipid bilayer, e.g., a membrane or viral envelope.

Embodiment 12. The protein of any of embodiments 1-11, wherein the F protein peptides are the same or different among the recombinant polypeptides of the protein.

Embodiment 13. The protein of any of embodiments 1-12, wherein the F protein peptide is directly fused to the C-terminal propeptide, or is linked to the C-terminal propeptide via a linker, such as a linker comprising glycine-X-Y repeats, wherein X and Y are independently any amino acid and optionally proline or hydroxyproline.

Embodiment 14. The protein of any of embodiments 1-13, which is soluble or does not directly bind to a lipid bilayer, e.g., a membrane or viral envelope.

Embodiment 15. The protein of any of embodiments 1-14, wherein the protein is capable of forming a rosette-like oligomer comprising F protein peptide trimers.

Embodiment 16. The protein of any of embodiments 1-15, wherein the protein is capable of binding to a cell surface attachment factor or receptor of a subject, optionally wherein the subject is a mammal such as a primate, e.g., human.

Embodiment 17. The protein of any of embodiments 1-16, wherein the C-terminal propeptide is of human collagen.

Embodiment 18. The protein of any of embodiments 1-17, wherein the C-terminal propeptide comprises a C-terminal polypeptide of proα1(I), proα1(II), proα1(III), proα1(V), proα1(XI), proα2(I), proα2(V), proα2(XI), or proα3(XI), or a fragment thereof.

Embodiment 19. The protein of any of embodiments 1-18, wherein the C-terminal propeptides are the same or different among the recombinant polypeptides.

Embodiment 20. The protein of any of embodiments 1-19, wherein the C-terminal propeptide comprises SEQ ID NO: 48 or an amino acid sequence at least 90% identical thereto capable of forming inter-polypeptide disulfide bonds and trimerizing the recombinant polypeptides.

Embodiment 21. The protein of any of embodiments 1-19, wherein the C-terminal propeptide comprises SEQ ID NO: 49 or an amino acid sequence at least 90% identical thereto capable of forming inter-polypeptide disulfide bonds and trimerizing the recombinant polypeptides.

Embodiment 22. The protein of any of embodiments 1-19, wherein the C-terminal propeptide comprises SEQ ID NO: 50 or an amino acid sequence at least 9/o identical thereto capable of forming inter-polypeptide disulfide bonds and trimerizing the recombinant polypeptides.

Embodiment 23. The protein of any of embodiments 1-19, wherein the C-terminal propeptide comprises SEQ ID NO: 51 or an amino acid sequence at least 90% identical thereto capable of forming inter-polypeptide disulfide bonds and trimerizing the recombinant polypeptides.

Embodiment 24. The protein of any of embodiments 1-19, wherein the C-terminal propeptide comprises SEQ ID NO: 52 or an amino acid sequence at least 90% identical thereto capable of forming inter-polypeptide disulfide bonds and trimerizing the recombinant polypeptides.

Embodiment 25. The protein of any of embodiments 1-19, wherein the C-terminal propeptide comprises SEQ ID NO: 53 or an amino acid sequence at least 90% identical thereto capable of forming inter-polypeptide disulfide bonds and trimerizing the recombinant polypeptides.

Embodiment 26. The protein of any of embodiments 1-19, wherein the C-terminal propeptide comprises SEQ ID NO: 54 or an amino acid sequence at least 90% identical thereto capable of forming inter-polypeptide disulfide bonds and trimerizing the recombinant polypeptides.

Embodiment 27. The protein of any of embodiments 1-19, wherein the C-terminal propeptide comprises any of SEQ ID NOs: 55-59 or an amino acid sequence at least 90% identical thereto capable of forming inter-polypeptide disulfide bonds and trimerizing the recombinant polypeptides.

Embodiment 28. The protein of any of embodiments 1-19, wherein the C-terminal propeptide comprises SEQ ID NO: 60 or an amino acid sequence at least 90% identical thereto capable of forming inter-polypeptide disulfide bonds and trimerizing the recombinant polypeptides.

Embodiment 29. The protein of any of embodiments 1-19, wherein the C-terminal propeptide comprises any of SEQ ID NOs: 61-63 or an amino acid sequence at least 90% identical thereto capable of forming inter-polypeptide disulfide bonds and trimerizing the recombinant polypeptides.

Embodiment 30. The protein of any of embodiments 1-29, wherein the C-terminal propeptide comprises an amino acid sequence comprising glycine-X-Y repeats linked to the N-terminus of any of SEQ ID NOs: 48-63, wherein X and Y are independently any amino acid and optionally proline or hydroxyproline, or an amino acid sequence at least 90% identical thereto capable of forming inter-polypeptide disulfide bonds and trimerizing the recombinant polypeptides.

Embodiment 31. The protein of any of embodiments 1-30, wherein the F protein peptide in each recombinant polypeptide is in a prefusion conformation or a postfusion conformation, optionally wherein the protein comprises a rosette-like oligomer comprising F protein peptide trimers as crutch-shaped rods.

Embodiment 32. The protein of any of embodiments 1-31, wherein the F protein peptide in each recombinant polypeptide comprises any of SEQ ID NOs: 17-47 or an amino acid sequence at least 80% identical thereto.

Embodiment 33. The protein of any of embodiments 1-31, wherein the recombinant polypeptide comprises any of SEQ ID NOs: 1-16 or an amino acid sequence at least 80% identical thereto.

Embodiment 34. An immunogen comprising the protein of any of embodiments 1-33.

Embodiment 35. A protein nanoparticle comprising the protein of any of embodiments 1-33 directly or indirectly linked to a nanoparticle.

Embodiment 36. A virus-like particle (VLP) comprising the protein of any of embodiments 1-33.

Embodiment 37. An isolated nucleic acid encoding one, two, three or more of the recombinant polypeptides of the protein of any of embodiments 1-33.

Embodiment 38. The isolated nucleic acid of embodiment 37, wherein a polypeptide encoding the F protein peptide is fused in-frame to a polypeptide encoding the C-terminal propeptide of collagen.

Embodiment 39. The isolated nucleic acid of embodiment 37 or 38, which is operably linked to a promoter.

Embodiment 40. The isolated nucleic acid of any of embodiments 37-39, which is a DNA molecule.

Embodiment 41. The isolated nucleic acid of any of embodiments 37-39, which is an RNA molecule, optionally an mRNA molecule such as a nucleoside-modified mRNA, a non-amplifying mRNA, a self-amplifying mRNA, or a trans-amplifying mRNA.

Embodiment 42. A vector comprising the isolated nucleic acid of any of embodiments 37-41.

Embodiment 43. The vector of embodiment 42, which is a viral vector.

Embodiment 44. A virus, a pseudovirus, or a cell comprising the vector of embodiment 42 or 43, optionally wherein the virus or cell has a recombinant genome.

Embodiment 45. An immunogenic composition comprising the protein, immunogen, protein nanoparticle, VLP, isolated nucleic acid, vector, virus, pseudovirus, or cell of any one of embodiments 1-44, and a pharmaceutically acceptable carrier.

Embodiment 46. A vaccine comprising the immunogenic composition of embodiment 45 and optionally an adjuvant, wherein the vaccine is optionally a subunit vaccine, and/or optionally wherein the vaccines is a prophylactic and/or therapeutic vaccine.

Embodiment 47. The vaccine of embodiment 46, wherein the vaccine comprises a plurality of different adjuvants.

Embodiment 48. A method of producing a protein, comprising: expressing the isolated nucleic acid or vector of any one of embodiments 37-43 in a host cell to produce the protein of any of embodiments 1-33; and purifying the protein.

Embodiment 49. The protein produced by the method of embodiment 48.

Embodiment 50. A method for generating an immune response to an F protein peptide or fragment or epitope thereof of an RSV in a subject, comprising administering to the subject an effective amount of the protein, immunogen, protein nanoparticle, VLP, isolated nucleic acid, vector, virus, pseudovirus, cell, immunogenic composition, or vaccine of any one of embodiments 1-47 and 49 to generate the immune response.

Embodiment 51. The method of embodiment 50, for treating or preventing infection with the RSV.

Embodiment 52. The method of embodiment 50 or 51, wherein generating the immune response inhibits or reduces replication of the RSV in the subject.

Embodiment 53. The method of any of embodiments 50-52, wherein the immune response comprises a cell-mediated response and/or a humoral response, optionally comprising production of one or more neutralizing antibody, such as a polyclonal antibody or a monoclonal antibody.

Embodiment 54. The method of any of embodiments 50-53, wherein the immune response is against the F protein peptide or fragment or epitope thereof of the RSV but not against the C-terminal propeptide.

Embodiment 55. The method of any of embodiments 50-54, wherein the administering does not lead to antibody dependent enhancement (ADE) in the subject due to prior exposure to one or more RSV.

Embodiment 56. The method of any of embodiments 50-55, wherein the administering does not lead to antibody dependent enhancement (ADE) in the subject when subsequently exposed to one or more RSV.

Embodiment 57. The method of any of embodiments 50-56, further comprising a priming step and/or a boosting step.

Embodiment 58. The method of any of embodiments 50-57, wherein the administering step is performed via topical, transdermal, subcutaneous, intradermal, oral, intranasal (e.g., intranasal spray), intratracheal, sublingual, buccal, rectal, vaginal, inhaled, intravenous (e.g., intravenous injection), intraarterial, intramuscular (e.g., intramuscular injection), intracardiac, intraosseous, intraperitoneal, transmucosal, intravitreal, subretinal, intraarticular, peri-articular, local, or epicutaneous administration.

Embodiment 59. The method of any of embodiments 50-58, wherein the effective amount is administered in a single dose or a series of doses separated by one or more interval.

Embodiment 60. The method of any of embodiments 50-59, wherein the effective amount is administered without an adjuvant.

Embodiment 61. The method of any of embodiments 50-59, wherein the effective amount is administered with an adjuvant.

Embodiment 62. A method comprising administering to a subject an effective amount of the protein of any one of embodiments 1-33 to generate in the subject a neutralizing antibody or neutralizing antisera to the RSV.

Embodiment 63. The method of embodiment 62, wherein the subject is a mammal, optionally a human or a non-human primate.

Embodiment 64. The method of embodiment 62 or 63, further comprising isolating the neutralizing antibody or neutralizing antisera from the subject.

Embodiment 65. The method of embodiment 64, further comprising administering an effective amount of the isolated neutralizing antibody or neutralizing antisera to a human subject via passive immunization to prevent or treat an infection by the RSV.

Embodiment 66. The method of any of embodiments 62-65, wherein the neutralizing antibody or neutralizing antisera to the RSV comprises polyclonal antibodies to the RSV F protein peptide or fragment or epitope thereof, optionally wherein the neutralizing antibody or neutralizing antisera is free or substantially free of antibodies to the C-terminal propeptide of collagen.

Embodiment 67. The method of any of embodiments 62-65, wherein the neutralizing antibody comprises a monoclonal antibody to the RSV F protein peptide or fragment or epitope thereof, optionally wherein the neutralizing antibody is free or substantially free of antibodies to the C-terminal propeptide of collagen.

Embodiment 68. The protein, immunogen, protein nanoparticle, VLP, isolated nucleic acid, vector, virus, pseudovirus, cell, immunogenic composition, or vaccine of any one of embodiments 1-47 and 49, for use in inducing an immune response to an RSV in a subject, and/or in treating or preventing an infection by the RSV.

Embodiment 69. Use of the protein, immunogen, protein nanoparticle, VLP, isolated nucleic acid, vector, virus, pseudovirus, cell, immunogenic composition, or vaccine of any one of embodiments 1-47 and 49, for inducing an immune response to an RSV in a subject, and/or for treating or preventing an infection by the RSV.

Embodiment 70. Use of the protein, immunogen, protein nanoparticle, VLP, isolated nucleic acid, vector, virus, pseudovirus, cell, immunogenic composition, or vaccine of any one of embodiments 1-47 and 49, for the manufacture of a medicament or a prophylactic for inducing an immune response to an RSV in a subject, and/or for treating or preventing an infection by the RSV.

Embodiment 71. A method for analyzing a sample, comprising: contacting a sample with the protein of any of embodiments 1-33, and detecting a binding between the protein and an analyte capable of specific binding to the F protein peptide or fragment or epitope thereof of the RSV.

Embodiment 72. The method of embodiment 71, wherein the analyte is an antibody, a receptor, or a cell recognizing the F protein peptide or fragment or epitope thereof.

Embodiment 73. The method of embodiment 71 or 72, wherein the binding indicates the presence of the analyte in the sample, and/or an infection by the RSV in a subject from which the sample is derived.

Embodiment 74. A kit comprising the protein of any of embodiments 1-33 and a substrate, pad, or vial containing or immobilizing the protein, optionally wherein the kit is an ELISA or lateral flow assay kit.

### EXAMPLES

The following examples are included for illustrative purposes only and are not intended to limit the scope of the invention.

### Example 1: Generation of Recombinant Polypeptides Comprising RSV F Protein Peptides

A secreted form of recombinant polypeptides comprising RSV F protein peptides as a candidate vaccine was generated.

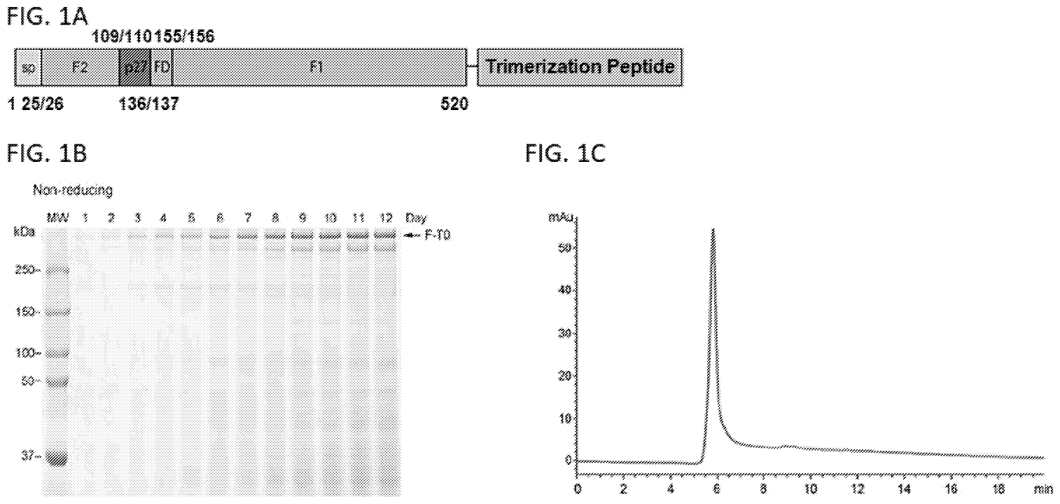

RSV F glycoprotein constructs were derived from RSV A2 strain (Accession No. AAC55970). The sequence encoding residues 1 to 520 of the F protein peptide were codon-optimized, synthesized and subcloned into a mammalian expression vector that encoded human C-propeptide of α1 collagen, at Hind III and Bgl II sites. FIG. 1A shows a schematic representation of an exemplary recombinant polypeptide.

The recombinant plasmid was transfected into GH-CHO (dfhr−) cells, selected without hypoxanthine thymine (HT) (Invitrogen), and stepwise gene amplified with increasing concentration of MTX (Sigma) for high titer expression of fusion protein under serum free culture using CD007-4 TM1 medium (Jianshun Biosciences). The exemplary recombinant polypeptides was initially purified via affinity binding to Endo180 using salt gradient elution, and further purified on a Superdex 200 gel filtration colume (GE Healthcare). The purity of the exemplary recombinant polypeptides comprising the RSV F peptide was determined by size-exclusive chromatography (SEC-HPLC) according to manufacturer's instructions (Sepax Technologies).

The production titer of disulfide bond-linked fusion peptides (e.g., trimers) was found to be up to about 0.15 g/L in a serum-free fed-batch culture process (FIG. 1B). Conditioned medium containing trimerized recombinant polypeptides was first purified via affinity binding to the Fc labelled collagen receptor uPARAP/Endo180, a member of the mannose receptor family (Thomas et al., (2005) J. Biol. Chem. 280, 22596-22605), which were pre-captured by a protein A chromatography column, followed by gel filtration chromatography. SEC-HPLC analysis indicated the purity of exemplary recombinant polypeptides trimers was about 95% (FIG. 1C).

The purified exemplary trimerized recombinant polypeptides (0.1 μg) were separated on 8% SDS-PAGE under non-reducing or reducing conditions and transferred to PVDF membranes. After blocking with 5% fat-free milk in PBS, the membranes were incubated with either mouse anti-RSV F monoclonal antibody (Millipore) or palivizumab (AbbVie) and rabbit anti-C-propeptide of type I procollagen (CICP) polyclonal antibody (Millipore). Two sg of purified exemplary recombinant polypeptides trimers were loaded for Coomassie blue staining. FIG. 2A shows that the fusions peptides are expressed as covalently-linked trimeric protein.

The structural features and integrity of this purified recombinant polypeptides was further confirmed by western blot analysis using antibodies specific to F and the trimerization peptide (FIG. 2A).

Purified recombinant polypeptide trimers were analyzed by negative staining electron microscopy. Purified recombinant polypeptides were diluted to 50 ug/mL and applied for 5 s onto carbon-coated 400 CU mesh grid that had been glow discharged at 12 mA for 20 s. The grids were negatively stained with 1% (w/v) uranyl formate for 20 s. The samples were collected through FEI Tecnai spirit electron microscopy operating 120 KeV, and micrographs were taken at 180,000× magnification. FIG. 2B shows recombinant polypeptide trimers aggregated in proteins, with macrostructures mainly in the form of rosette-like oligomers, which are similar to observations made on full-length F proteins (Calder et al., (2000) Virology 271, 122-131; Smith et al., (2012) PloS One 7, e50852). The molecules in the rosettes were crutch-shaped rods with their wider ends projecting away from the centers, consistent with the reported postfusion conformation of F (Swanson et al., (2011) Prot. Natl. Acad. Sci. U.S.A. 108, 9619-9624).

The affinity binding of monoclonal antibody palivizumab to exemplary recombinant polypeptide trimers was measured by fortebio OCTET QKe system (Pall) using biolayer interferometry (BLI). Palivizumab at 5 μg/mL was directly immobilized on Protein A sensors, then balanced in PBS and placed into wells containing 2-fold dilutions of fusion peptides (starting at 20 μg/mL). Disassociation was carried out by dipping into PBS and the data was processed using Data Analyze software to 1:1 binding model by subtracting buffer reference.

The binding affinity of palivizumab to purified exemplary recombinant polypeptides exhibited a K_{D} less than one picomolor (FIG. 2C), which indicates that antigenic site II is exposed on the exemplary fusion peptides.

### Example 2: Functional Characterization of Recombinant Polypeptides Comprising RSV F Protein Peptides

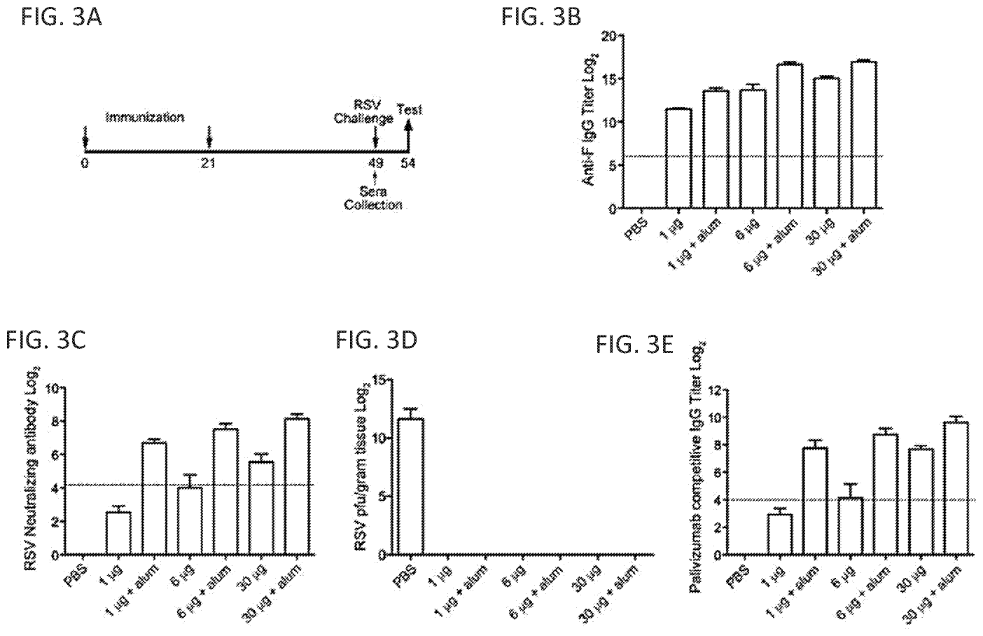

To evaluate the immunogenicity and protective efficacy of exemplary recombinant polypeptides generated as described in Example 1, randomly grouped BALB/c mice were immunized i.m. twice on day 0 and 21 with one of three doses (1, 6 and 30 μg) of exemplary fusion peptides with or without alum-absorbed (Imject alum adjuvant (Thermo Scientifc)). An additional group immunized with PBS served as a control. Sera were collected on day 49 before intranasal (i.n.) challenge with 1×10⁶ pfu RSV A2 strain (FIG. 3A). Animals were observed daily and euthanized on day 54 for lung tissue collection.

Sera were evaluated in an enzyme linked immunosorbent assay (ELISA). Briefly, 96-well plates were coated with 2 μg/mL of purified exemplary fusion peptides (in PBS) overnight at 4° C. and blocked with 1 mg/mL BSA. The plates were washed with PBST and subsequently incubated with serial 2-fold dilutions (1:64 to 1:262,144) of serum for 2 hr at RT. Bound antibodies were detected by HRP-conjugated goat anti-mouse IgG (SouthernBiotech) for 1 hr at RT. The enzymatic reaction was developed with TMB (Thermo) and stopped by addition of 2M HCl, and the absorbance at 450 nm was recorded. Sera of PBS-immunized mice were used as negative control at the same dilution, and antibody titer was defined as the serum dilution that resulted in the ratio of OD RSV F-Trimer and OD PBS at 2.0.

Serum analysis revealed that all groups immunized with exemplary recombinant polypeptides had high levels of RSV F protein peptide-specific antibodies, and exhibited dose- and adjuvant-dependent manner (FIG. 3B). The neutralizing activity of anti-F antibodies was measured by a microneutralization assay.

The RSV microneutralization assay was performed using HeLa cells and the RSV A2 strain. Sera were heated inactivated at 56° C. for 30 min, and serially diluted in serum-free DMEM in 96-well cell culture plates (50 μL/well). An equal volume of virus (1,000 pfu/mL prepared in serum-free DMEM) was added into the plates and the sera/virus mixture was incubated for 1 hr at 37° C. Approximately 5×10⁴ HeLa cells in 100 μL DMEM supplemented with 10% FBS were added into the plates and incubated at 37° C. until positive control (virus only) wells show 100% CPE. Plates were washed with PBST and fixed with 80% pre-cold acetone in PBS for 10 min. Palivizumab at 100 ng/mL was added into the wells and incubated for 2 hr at RT after blocking for 1 hr by 1 mg/mL BSA. After three times washing, HRP-conjugated goat anti-human IgG (SouthernBiotech) was added, the enzymatic reaction was developed, and ODs were recorded at 450 nm. The dilution resulted in 50% inhibition of CPE formation was identified as the neutralizing antibody titer.

The microneutralization assay showed that exemplary recombinant polypeptide-induced anti-F antibodies possessed potent RSV neutralizing activity, and co-injection of alum adjuvant elicited higher neutralizing antibody titer under the same dose of the exemplary recombinant polypeptides. These results were consistent with the results of anti-F antibodies (FIG. 3C).

The protective efficacy of exemplary recombinant polypeptides was assessed by determining the viral replication in the lungs on day 5 after virus challenge. Mice were sacrificed five days after intranasal RSV challenge, and harvested left lungs were weighed and homogenized in 1 mL serum-free DMEM. The homogenate was clarified by centrifugation at 1,000×g for 10 min at 4° C. and virus in the lung samples was tittered by plaque assay according to the method mentioned above.

The results showed that all exemplary recombinant polypeptides immunized mice were completely protected from RSV replication and no virus could be detected, whereas the PBS immunized control group displayed high levels of virus load in lungs (FIG. 3D).

Since antigenic site II is exposed on the exemplary recombinant polypeptides (see, Example 1 and FIG. 2C), a palivizumab competitive ELISA was performed to determine whether antibodies elicited by the exemplary recombinant polypeptides were direct against this site.

The palivizumab competitive ELISA was performed using 96-well ELISA plates coated with 5×10⁶ pfu/mL heat-inactivated RSV (HI-RSV, in 50 mM carbonate-bicarbonate buffer, pH 9.2) and incubated overnight at 4° C. The uncoated surfaces were blocked with 1 mg/mL BSA. Two-fold dilutions (1:32 to 1:4,096) of serum mixture with 100 ng/mL of palivizumab were added to the wells and incubated for 2 hr at RT. Bound palivizumab was detected by using HRP-conjugated goat anti-human IgG (SouthernBiotech) and TMB substrate. Wells containing the sera of PBS-immunized mice represented the un-competed positive control and percent of inhibition was calculated as ((OD PBS−OD RSV F-trimer)/OD PBS)×100%. Competitive binding titers were expressed as the dilution resulted in 50% inhibition.

Inhibition of palivizumab binding to heat-inactivated RSV (HI-RSV) particles was observed with all serum samples collected from mice immunized with RSV F-Trimer either in presence or absence of alum adjuvant (FIG. 3E). These results demonstrated that neutralizing antibodies elicited by exemplary fusion peptide immunization could effectively prevent RSV replication in the lungs of challenged mice at least through targeting antigenic site II of virus.

Since F1-RSV vaccination unexpectedly led to enhanced disease severity (Kim et al., (1969) Am. J, Epidemiol. 89, 422-434; Chin et al., (1969) Am. J. Epidemiol. 89, 449-463), safety monitoring is a top priority in RSV candidate vaccine development (Murata, Y. (2009) Clin. Lab. Med. 29, 725-739). Histological examination of lung tissues obtained from immunized mice 5 days after challenge was performed to evaluate the safety of exemplary recombinant polypeptides.

Harvested right lung tissues were fixed in 10%/c netural buffered formalin, paraffin-embedded, and sectioned at 5 μm and stained with H&E for histopathological evaluations. Pictures were captured at 200× magnification under Nikon microscope.

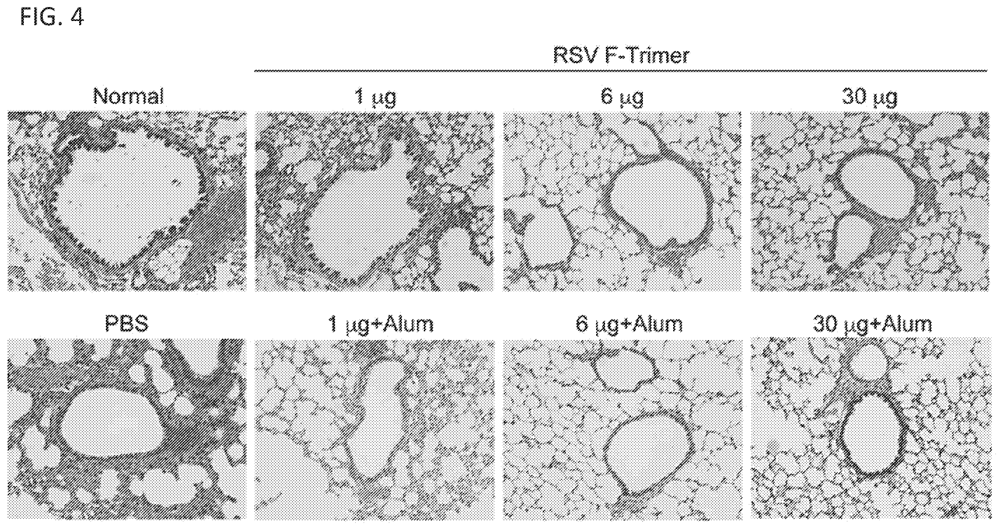

H&E staining revealed that the PBS immunized control group exhibited some degree of alveolitis, peribronchiolitis and perivasculitis, and accompanied by significant inflammatory cells infiltration (FIG. 4). In contrast, a limited immune cell infiltration and no obvious pathological changes have taken place in any dose of exemplary recombinant polypeptides immunized animals whether with or without alum adjuvant (FIG. 4). This result is supportive of exemplary recombinant polypeptide immunization to protect from the vaccine-mediated enhanced disease after RSV infection.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| SEQ ID NO. | SEQUENCE | DESCRIPTION |
|---|---|---|
| 1 | QNITEEFYQSTCSAVSKGYLSALRTGWYTSVITIELSNIKENKCNGTDAKVKLIKQ | RSV fusion |
| | ELDKYKNAVTELQLLMQSTPATNNRARRELPRTMNYTLNNAKKTNVTLSKKRKRRF | glycoprotein F- |
| | LGFLLGVGSAIASGVAVSKVLHLEGEVNKIKSALLSTNKAVVSLSNGVSVLTSKVL | Trimer fusion |
| | DLKNYIDKQLLPIVNKQSCSISNIATVIEFQQKNNRLLEITREFSVNAGVTTPVST | polypeptide |
| | YMLTNSELLSLINDMPITNDQKKLMSNNVQIVRQQSYSIMSIIKEEVLAYVVQLPL | without signal |
| | YGVIDTPCWKLHTSPLCTTNTKEGSNICLTRTDRGWYCDNAGSVSFFPQAETCKVQ | peptide, 806 aa |
| | SNRVFCDTMNSLTLPSEVNLCNVDIFNPKYDCKIMTSKTDVSSSVITSLGAIVSCY | |
| | GKTKCTASNKNRGIIKTFSNGCDYVSNKGVDTVSVGNTLYYVNKQEGKSLYVKGEP | |
| | IINFYDPLVFPSDEFDASISQVNEKINQSLAFIRKSDELLHNVNAGKRSNGLPGPI | |
| | GPPGPRGRTGDAGPVGPPGPPGPPGPPGPPSAGFDFSFLPQPPQEKAHDGGRYYRA | |
| | NDANVVRDRDLEVDTTLKSLSQQIENIRSPEGSRKNPARTCRDLKMCHSDWKSGEY | |
| | WIDPNQGCNLDAIKVFCNMETGETCVYPTQPSVAQKNWYISKNPKDKRHVWFGESM | |
| | TDGFQFEYGGQGSDPADVAIQLTFLRLMSTEASQNITYHCKNSVAYMDQQTGNLKK | |
| | ALLLQGSNEIEIRAEGNSRFTYSVTVDGCTSHTGAWGKTVIEYKTTKTSRLPIIDV | |
| | APLDVGAPDQEFGFDVGPVCFL | |
| 2 | MELLILKANAITTILTAVTFCFASGQNITEEFYQSTCSAVSKGYLSALRTGWYTSV | RSV fusion |
| | ITIELSNIKENKCNGTDAKVKLIKQELDKYKNAVTELQLLMQSTPATNNRARRELP | glycoprotein F- |
| | RFMNYTLNNAKKTNVTLSKKRKRRFLGFLLGVGSAIASGVAVSKVLHLEGEVNKIK | Trimer fusion |
| | SALLSTNKAVVSLSNGVSVLTSKVLDLKNYIDKQLLPIVNKQSCSISNIATVIEFQ | polypeptide |
| | QKNNRLLEITREFSVNAGVTTPVSTYMLTNSELLSLINDMPITNDQKKLMSNNVQI | with signal |
| | VRQQSYSIMSIIKEEVLAYVVQLPLYGVIDTPCWKLHTSPLCTTNTKEGSNICLTR | peptide, 831 aa |
| | TDRGWYCDNAGSVSFFPQAETCKVQSNRVFCDTMNSLTLPSEVNLCNVDIFNPKYD | |
| | CKIMTSKTDVSSSVITSLGAIVSCYGKTKCTASNKNRGIIKTFSNGCDYVSNKGVD | |
| | TVSVGNTLYYVNKQEGKSLYVKGEPIINFYDPLVFPSDEFDASISQVNEKINQSLA | |
| | FIRKSDELLHNVNAGKRSNGLPGPIGPPGPRGRTGDAGPVGPPGPPGPPGPPGPPS | |
| | AGFDESFLPQPPQEKAHDGGRYYRANDANVVRDRDLEVDTTLKSLSQQIENIRSPE | |
| | GSRKNPARTCRDLKMCHSDWKSGEYWIDPNQGCNLDAIKVFCNMETGETCVYPTQP | |
| | SVAQKNWYISKNPKDKRHVWFGESMTDGFQFEYGGQGSDPADVAIQLTFLRLMSTE | |
| | ASQNITYHCKNSVAYMDQQTGNLKKALLLQGSNEIEIRAEGNSRFTYSVTVDGCTS | |
| | HTGAWGKTVIEYKTTKTSRLPIIDVAPLDVGAPDQEFGFDVGPVCFL | |
| 3 | QNITEEFYQSTCSAVSKGYLSALRTGWYTSVITIELSNIKENKCNGIDAKVKLIKQ | RSV fusion |
| | ELDKYKNAVTELQLLMQSTPATNNRARAELPRFMNYTLNNAKKTNVTLSKKRKRRF | glycoprotein F- |
| | LGFLLGVGSAIASGVAVSKVLHLEGEVNKIKSALLSTNKAVVSLSNGVSVLTSKVL | Trimer fusion |
| | DLKNYIDKQLLPIVNKQSCSISNIATVIEFQQKNNRLLEITREFSVNAGVTTPVST | polypeptide |
| | YMLTNSELLSLINDMPITNDQKKLMSNNVQIVRQQSYSIMSIIKEEVLAYVVQLPL | without signal |
| | YGVIDTPCWKLHTSPLCTTNTKEGSNICLTRTDRGWYCDNAGSVSFFPQAETCKVQ | peptide, 806 aa |
| | SNRVFCDTMNSLTLPSEVNLCNVDIFNPKYDCKIMTSKTDVSSSVITSLGAIVSCY | (R109A Mutant) |
| | GKTKCTASNKNRGIIKTFSNGCDYVSNKGVDTVSVGNTLYYVNKQEGKSLYVKGEP | |
| | IINFYDPLVFPSDEFDASISQVNEKINQSLAFIRKSDELLHNVNAGKRSNGLPGPI | |
| | GPPGPRGRTGDAGPVGPPGPPGPPGPPGPPSAGFDFSFLPQPPQEKAHDGGRYYRA | |
| | NDANVVRDRDLEVDTTLKSLSQQIENIRSPEGSRKNPARTCRDLKMCHSDWKSGEY | |
| | WIDPNQGCNLDAIKVFCNMETGETCVYPTQPSVAQKNWYISKNPKDKRHVWFGESM | |
| | TDGFQFEYGGQGSDPADVAIQLTFLRLMSTEASQNITYHCKNSVAYMDQQTGNLKK | |
| | ALLLQGSNEIEIRAEGNSRFTYSVTVDGCTSHTGAWGKTVIEYKTTKTSRLPIIDV | |
| | APLDVGAPDQEFGFDVGPVCFL | |
| 4 | MELLILKANAITTILTAVTFCFASGQNITEEFYQSTCSAVSKGYLSALRTGWYTSV | RSV fusion |
| | ITIELSNIKENKCNGTDAKVKLIKQELDKYKNAVTELQLLMQSTPATNNRARAELP | glycoprotein F- |
| | RFMNYTLNNAKKTNVTLSKKRKRRFLGFLLGVGSAIASGVAVSKVLHLEGEVNKIK | Trimer fusion |
| | SALLSTNKAVVSLSNGVSVLTSKVLDLKNYIDKQLLPIVNKQSCSISNIATVIEFQ | polypeptide |
| | QKNNRLLEITREFSVNAGVTTPVSTYMLTNSELLSLINDMPITNDQKKLMSNNVQI | with signal |
| | VRQQSYSIMSIIKEEVLAYVVQLPLYGVIDTPCWKLHTSPLCTTNTKEGSNICLTR | peptide, 831 aa |
| | TDRGWYCDNAGSVSFFPQAETCKVQSNRVFCDTMNSLTLPSEVNLCNVDIFNPKYD | (R109A Mutant) |
| | CKIMTSKTDVSSSVITSLGAIVSCYGKTKCTASNKNRGIIKTFSNGCDYVSNKGVD | |
| | TVSVGNTLYYVNKQEGKSLYVKGEPIINFYDPLVFPSDEFDASISQVNEKINQSLA | |
| | FIRKSDELLHNVNAGKRSNGLPGPIGPPGPRGRTGDAGPVGPPGPPGPPGPPGPPS | |
| | AGFDFSFLPQPPQEKAHDGGRYYRANDANVVRDRDLEVDTTLKSLSQQIENIRSPE | |
| | GSRKNPARTCRDLKMCHSDWKSGEYWIDPNQGCNLDAIKVFCNMETGETCVYPTQP | |
| | SVAQKNWYISKNPKDKRHVWFGESMTDGFQFEYGGQGSDPADVAIQLTFLRLMSTE | |
| | ASQNITYHCKNSVAYMDQQTGNLKKALLLQGSNEIEIRAEGNSRFTYSVTVDGCTS | |
| | HTGAWGKTVIEYKTTKTSRLPIIDVAPLDVGAPDQEFGFDVGPVCEL | |
| 5 | QNITEEFYQSTCSAVSKGYLSALRTGWYTSVITIELSNIKENKCNGTDAKVKLIKQ | RSV fusion |
| | ELDKYKNAVTELQLLMQSTPATNNRARRELPREMNYTLNNAKKTNVTLSKKRKRAF | glycoprotein F- |
| | LGFLLGVGSAIASGVAVSKVLHLEGEVNKIKSALLSTNKAVVSLSNGVSVLTSKVL | Trimer fusion |
| | DLKNYIDKQLLPIVNKQSCSISNIATVIEFQQKNNRLLEITREFSVNAGVTTPVST | polypeptide |
| | YMLTNSELLSLINDMPITNDQKKLMSNNVQIVRQQSYSIMSLIKEEVLAYVVQLPL | without signal |
| | YGVIDTPCWKLHTSPLCTTNTKEGSNICLTRTDRGWYCDNAGSVSFFPQAETCKVQ | peptide, 806 aa |
| | SNRVFCDTMNSLTLPSEVNLCNVDIFNPKYDCKIMTSKTDVSSSVITSLGAIVSCY | (R136A Mutant) |
| | GKTKCTASNKNRGIIKTFSNGCDYVSNKGVDTVSVGNTLYYVNKQEGKSLYVKGEP | |
| | IINFYDPLVFPSDEFDASISQVNEKINQSLAFIRKSDELLHNVNAGKRSNGLPGPI | |
| | GPPGPRGRTGDAGPVGPPGPPGPPGPPGPPSAGFDFSFLPQPPQEKAHDGGRYYRA | |
| | NDANVVRDRDLEVDTTLKSLSQQIENIRSPEGSRKNPARTCRDLKMCHSDWKSGEY | |
| | WIDPNQGCNLDAIKVFCNMETGETCVYPTQPSVAQKNWYISKNPKDKRHVWFGESM | |
| | TDGFQFEYGGQGSDPADVAIQLTFLRLMSTEASQNITYHCKNSVAYMDQQTGNLKK | |
| | ALLLQGSNEIEIRAEGNSRFTYSVTVDGCTSHTGAWGKTVIEYKTTKTSRLPIIDV | |
| | APLDVGAPDQEFGFDVGPVCFL | |
| 6 | MELLILKANAITTILTAVTFCFASGQNITEEFYQSTCSAVSKGYLSALRTGWYTSV | RSV fusion |
| | ITIELSNIKENKCNGTDAKVKLIKQELDKYKNAVTELQLLMQSTPATNNRARRELP | glycoprotein F- |
| | RFMNYTLNNAKKTNVTLSKKRKRAFLGFLLGVGSAIASGVAVSKVLHLEGEVNKIK | Trimer fusion |
| | SALLSTNKAVVSLSNGVSVLTSKVLDLKNYIDKQLLPIVNKQSCSISNIATVIEFQ | polypeptide |
| | QKNNRLLEITREFSVNAGVTTPVSTYMLTNSELLSLINDMPITNDQKKLMSNNVQI | with signal |
| | VRQQSYSIMSIIKEEVLAYVVQLPLYGVIDTPCWKLHTSPLCTTNTKEGSNICLTR | peptide, 831 aa |
| | TDRGWYCDNAGSVSFFPQAETCKVQSNRVFCDTMNSLTLPSEVNLCNVDIFNPKYD | (R136A Mutant) |
| | CKIMTSKTDVSSSVITSLGAIVSCYGKTKCTASNKNRGIIKTFSNGCDYVSNKGVD | |
| | TVSVGNTLYYVNKQEGKSLYVKGEPIINFYDPLVFPSDEFDASISQVNEKINQSLA | |
| | FIRKSDELLHNVNAGKRSNGLPGPIGPPGPRGRTGDAGPVGPPGPPGPPGPPGPPS | |
| | AGFDFSFLPQPPQEKAHDGGRYYRANDANVVRDRDLEVDTTLKSLSQQIENIRSPE | |
| | GSRKNPARTCRDLKMCHSDWKSGEYWIDPNQGCNLDAIKVFCNMETGETCVYPTQP | |
| | SVAQKNWYISKNPKDKRHVWEGESMTDGFQFEYGGQGSDPADVAIQLTFLRLMSTE | |
| | ASQNITYHCKNSVAYMDQQTGNLKKALLLQGSNEIEIRAEGNSRFTYSVTVDGCTS | |
| | HTGAWGKTVIEYKTTKTSRLPIIDVAPLDVGAPDQEFGFDVGPVCFL | |
| 7 | QNITEEFYQSTCSAVSKGYLSALRTGWYTSVITIELSNIKENKCNGTDAKVKLIKQ | RSV fusion |
| | ELDKYKNAVTELQLLMQSTPATNNRARAELPREMNYTLNNAKKTNVTLSKKRKRAF | glycoprotein F- |
| | LGFLLGVGSAIASGVAVSKVLHLEGEVNKIKSALLSTNKAVVSLSNGVSVLTSKVL | Trimer fusion |
| | DLKNYIDKQLLPIVNKQSCSISNIATVIEFQQKNNRLLEITREFSVNAGVTTPVST | polypeptide |
| | YMLTNSELLSLINDMPITNDQKKLMSNNVQIVRQQSYSIMSIIKEEVLAYVVQLPL | without signal |
| | YGVIDTPCWKLHTSPLCTTNTKEGSNICLTRTDRGWYCDNAGSVSFFPQAETCKVQ | peptide, 806 aa |
| | SNRVFCDTMNSLTLPSEVNLCNVDIFNPKYDCKIMTSKTDVSSSVITSLGAIVSCY | (R109A/R136A |
| | GKTKCTASNKNRGIIKTFSNGCDYVSNKGVDTVSVGNTLYYVNKQEGKSLYVKGEP | Mutant) |
| | IINFYDPLVFPSDEFDASISQVNEKINQSLAFIRKSDELLHNVNAGKRSNGLPGPI | |
| | GPPGPRGRTGDAGPVGPPGPPGPPGPPGPPSAGFDFSFLPQPPQEKAHDGGRYYRA | |
| | NDANVVRDRDLEVDTTLKSLSQQIENIRSPEGSRKNPARTCRDLKMCHSDWKSGEY | |
| | WIDPNQGCNLDAIKVFCNMETGETCVYPTQPSVAQKNWYISKNPKDKRHVWFGESM | |
| | TDGFQFEYGGQGSDPADVAIQLTFLRLMSTEASQNITYHCKNSVAYMDQQTGNLKK | |
| | ALLLQGSNEIEIRAEGNSRFTYSVTVDGCTSHTGAWGKIVIEYKTTKTSRLPIIDV | |
| | APLDVGAPDQEFGFDVGPVCFL | |
| 8 | MELLILKANAITTILTAVTFCFASGQNITEEFYQSTCSAVSKGYLSALRTGWYTSV | RSV fusion |
| | ITIELSNIKENKCNGTDAKVKLIKQELDKYKNAVTELQLLMQSTPATNNRARAELP | glycoprotein F- |
| | RFMNYTLNNAKKTNVTLSKKRKRAFLGFLLGVGSAIASGVAVSKVLHLEGEVNKIK | Trimer fusion |
| | SALLSTNKAVVSLSNGVSVLTSKVLDLKNYIDKQLLPIVNKQSCSISNIATVIEFQ | polypeptide |
| | QKNNRLLEITREFSVNAGVTTPVSTYMLTNSELLSLINDMPITNDQKKLMSNNVQI | with signal |
| | VRQQSYSIMSIIKEEVLAYVVQLPLYGVIDTPCWKLHTSPLCTTNTKEGSNICLTR | peptide, 831 aa |
| | TDRGWYCDNAGSVSFFPQAETCKVQSNRVFCDTMNSLTLPSEVNLCNVDIFNPKYD | (R109A/R136A |
| | CKIMTSKTDVSSSVITSLGAIVSCYGKTKCTASNKNRGIIKTFSNGCDYVSNKGVD | Mutant) |
| | TVSVGNTLYYVNKQEGKSLYVKGEPIINFYDPLVFPSDEFDASISQVNEKINQSLA | |
| | FIRKSDELLHNVNAGKRSNGLPGPIGPPGPRGRIGDAGPVGPPGPPGPPGPPGPPS | |
| | AGFDFSFLPQPPQEKAHDGGRYYRANDANVVRDRDLEVDTTLKSLSQQIENIRSPE | |
| | GSRKNPARTCRDLKMCHSDWKSGEYWIDPNQGCNLDAIKVFCNMETGETCVYPTQP | |
| | SVAQKNWYISKNPKDKRHVWFGESMTDGFQFEYGGQGSDPADVAIQLTFLRLMSTE | |
| | ASQNITYHCKNSVAYMDQQTGNLKKALLLQGSNEIEIRAEGNSRFTYSVTVDGCTS | |
| | HTGAWGKTVIEYKTTKTSRLPIIDVAPLDVGAPDQEFGFDVGPVCFL | |
| 9 | QNITEEFYQSTCSAVSKGYLSALRTGWYTSVITIELSNIKENKCNGTDAKVKLIKQ | RSV fusion |
| | ELDKYKNAVTELQLLMQSTPATNNRARRELPRFMNYTLNNAKKTNVTLSKKRKRRF | glycoprotein F- |
| | LGFLLGVGSAIASGVAVSKVLHLPGEVNKIKSALLSTNKAVVSLSNGVSVLTSKVL | Trimer fusion |
| | DLKNYIDKQLLPIVNKQSCSIPNIATVIEFQQKNNRLLEITREFSVNAGVTTPVST | polypeptide |
| | YMLTNSELLSLINDMPITNDQKKLMSNNVQIVRQQSYSIMSIIKEEVLAYVVQLPL | without signal |
| | YGVIDTPCWKLHTSPLCTTNTKEGSNICLTRTDRGWYCDNAGSVSFFPQAETCKVQ | peptide, 806 aa |
| | SNRVFCDTMNSLTLPSEVNLCNVDIFNPKYDCKIMTSKTDVSSSVITSLGAIVSCY | (E161P/S215P |
| | GKTKCTASNKNRGIIKTFSNGCDYVSNKGVDTVSVGNTLYYVNKQEGKSLYVKGEP | Mutant) |
| | IINFYDPLVFPSDEFDASISQVNEKINQSLAFIRKSDELLHNVNAGKRSNGLPGPI | |
| | GPPGPRGRIGDAGPVGPPGPPGPPGPPGPPSAGFDFSFLPQPPQEKAHDGGRYYRA | |
| | NDANVVRDRDLEVDTTLKSLSQQIENIRSPEGSRKNPARTCRDLKMCHSDWKSGEY | |
| | WIDPNQGCNLDAIKVFCNMETGETCVYPTQPSVAQKNWYISKNPKDKRHVWFGESM | |
| | TDGFQFEYGGQGSDPADVAIQLTFLRLMSTEASQNITYHCKNSVAYMDQQTGNLKK | |
| | ALLLQGSNEIEIRAEGNSRFTYSVTVDGCTSHTGAWGKTVIEYKTTKTSRLPIIDV | |
| | APLDVGAPDQEFGFDVGPVCFL | |
| 10 | MELLILKANAITTILTAVTFCFASGQNITEEFYQSTCSAVSKGYLSALRTGWYTSV | RSV fusion |
| | ITIELSNIKENKCNGTDAKVKLIKQELDKYKNAVTELQLLMQSTPATNNRARRELP | glycoprotein F- |
| | RFMNYTLNNAKKTNVTLSKKRKRRFLGFLLGVGSAIASGVAVSKVLHLPGEVNKIK | Trimer fusion |
| | SALLSTNKAVVSLSNGVSVLTSKVLDLKNYIDKQLLPIVNKQSCSIPNIATVIEFQ | polypeptide |
| | QKNNRLLEITREFSVNAGVTTPVSTYMLTNSELLSLINDMPITNDQKKLMSNNVQI | with signal |
| | VRQQSYSIMSILKEEVLAYVVQLPLYGVIDTPCWKLHTSPLCTTNTKEGSNICLTR | peptide, 831 aa |
| | TDRGWYCDNAGSVSFFPQAETCKVQSNRVFCDTMNSLTLPSEVNLCNVDIFNPKYD | (E161P/S215P |
| | CKIMTSKTDVSSSVITSLGAIVSCYGKTKCTASNKNRGIIKTFSNGCDYVSNKGVD | Mutant) |
| | TVSVGNTLYYVNKQEGKSLYVKGEPIINFYDPLVFPSDEFDASISQVNEKINQSLA | |
| | FIRKSDELLHNVNAGKRSNGLPGPIGPPGPRGRTGDAGPVGPPGPPGPPGPPGPPS | |
| | AGFDFSFLPQPPQEKAHDGGRYYRANDANVVRDRDLEVDTTLKSLSQQIENIRSPE | |
| | GSRKNPARTCRDLKMCHSDWKSGEYWIDPNQGCNLDAIKVFCNMETGETCVYPTQP | |
| | SVAQKNWYISKNPKDKRHVWFGESMTDGFQFEYGGQGSDPADVAIQLTFLRLMSTE | |
| | ASQNITYHCKNSVAYMDQQTGNLKKALLLQGSNEIEIRAEGNSRFTYSVTVDGCTS | |
| | HTGAWGKTVIEYKTTKISRLPIIDVAPLDVGAPDQEFGFDVGPVCFL | |
| 11 | QNITEEFYQSTCSAVSKGYLSALRTGWYTSVITIELSNIKENKCNGTDAKVKLIKQ | RSV fusion |
| | ELDKYKNAVTELQLLMQSTPATNNRARAELPREMNYTLNNAKKTNVTLSKKRKRAF | glycoprotein F- |
| | LGFLLGVGSAIASGVAVSKVLHLPGEVNKIKSALLSTNKAVVSLSNGVSVLTSKVL | Trimer fusion |
| | DLKNYIDKQLLPIVNKQSCSIPNIATVIEFQQKNNRLLEITREFSVNAGVTTPVST | polypeptide |
| | YMLTNSELLSLINDMPITNDQKKLMSNNVQIVRQQSYSIMSIIKEEVLAYVVQLPL | without signal |
| | YGVIDTPCWKLHTSPLCTTNTKEGSNICLTRTDRGWYCDNAGSVSFFPQAETCKVQ | peptide, 806 aa |
| | SNRVFCDTMNSLTLPSEVNLCNVDIFNPKYDCKIMTSKTDVSSSVITSLGAIVSCY | (R109A/R136A/ |
| | GKTKCTASNKNRGIIKTFSNGCDYVSNKGVDTVSVGNTLYYVNKQEGKSLYVKGEP | E161P/S215P |
| | IINFYDPLVFPSDEFDASISQVNEKINQSLAFIRKSDELLHNVNAGKRSNGLPGPI | Mutant) |
| | GPPGPRGRTGDAGPVGPPGPPGPPGPPGPPSAGFDFSFLPQPPQEKAHDGGRYYRA | |
| | NDANVVRDRDLEVDTTLKSLSQQIENIRSPEGSRKNPARTCRDLKMCHSDWKSGEY | |
| | WIDPNQGCNLDAIKVFCNMETGETCVYPTQPSVAQKNWYISKNPKDKRHVWFGESM | |
| | TDGFQFEYGGQGSDPADVAIQLTFLRLMSTEASQNITYHCKNSVAYMDQQTGNLKK | |
| | ALLLQGSNEIEIRAEGNSRFTYSVTVDGCTSHTGAWGKTVIEYKTTKTSRLPIIDV | |
| | APLDVGAPDQEFGFDVGPVCFL | |
| 12 | MELLILKANAITTILTAVTFCFASGQNITEEFYQSTCSAVSKGYLSALRTGWYTSV | RSV fusion |
| | ITIELSNIKENKCNGTDAKVKLIKQELDKYKNAVTELQLLMQSTPATNNRARAELP | glycoprotein F- |
| | REMNYTLNNAKKTNVTLSKKRKRAFLGFLLGVGSAIASGVAVSKVLHLPGEVNKIK | Trimer fusion |
| | SALLSTNKAVVSLSNGVSVLTSKVLDLKNYIDKQLLPIVNKQSCSIPNIATVIEFQ | polypeptide |
| | QKNNRLLEITREFSVNAGVTTPVSTYMLTNSELLSLINDMPITNDQKKLMSNNVQI | with signal |
| | VRQQSYSIMSIIKEEVLAYVVQLPLYGVIDTPCWKLHTSPLCTTNTKEGSNICLTR | peptide, 831 aa |
| | TDRGWYCDNAGSVSFFPQAETCKVQSNRVFCDTMNSLTLPSEVNLCNVDIFNPKYD | (R109A/R136A/ |
| | CKIMTSKTDVSSSVITSLGAIVSCYGKTKCTASNKNRGIIKTFSNGCDYVSNKGVD | E161P/S215P |
| | TVSVGNTLYYVNKQEGKSLYVKGEPIINFYDPLVFPSDEFDASISQVNEKINQSLA | Mutant) |
| | FIRKSDELLHNVNAGKRSNGLPGPIGPPGPRGRTGDAGPVGPPGPPGPPGPPGPPS | |
| | AGFDFSFLPQPPQEKAHDGGRYYRANDANVVRDRDLEVDTTLKSLSQQIENIRSPE | |
| | GSRKNPARTCRDLKMCHSDWKSGEYWIDPNQGCNLDAIKVFCNMETGETCVYPTQP | |
| | SVAQKNWYISKNPKDKRHVWFGESMTDGFQFEYGGQGSDPADVAIQLTFLRLMSTE | |
| | ASQNITYHCKNSVAYMDQQTGNLKKALLLQGSNEIEIRAEGNSRFTYSVTVDGCTS | |
| | HTGAWGKTVIEYKTTKTSRLPIIDVAPLDVGAPDQEFGFDVGPVCFL | |
| 13 | QNITEEFYQSTCSAVSKGYLSALRTGWYTSVITIELSNIKENKCNGTDAKVKLIKQ | RSV fusion |
| | ELDKYKNAVTELQLLMQSTPATNNRARAELPREMNYTLNNAKKTNVTLSKKRKRAF | glycoprotein F- |
| | LGFLLGVGSAIASGVAVSKVLHLPGEVNKIKSALLSTNKAVVSLSNGVSVLTSKVL | Trimer fusion |
| | DLKNYIDKQLLPIVNKQSCSIPNIATVIEFQQKNNRLLEITREFSVNAGVTTPVST | polypeptide F- |
| | YMLTNSELLSLINDMPITNDQKKLMSNNVQIVRQQSYSIMSIIKEEVLAYVVQLPL | CICP (no |
| | YGVIDTPCWKLHTSPLCTTNTKEGSNICLTRTDRGWYCDNAGSVSFFPQAETCKVQ | glycine repeat |
| | SNRVFCDTMNSLTLPSEVNLCNVDIFNPKYDCKIMTSKTDVSSSVITSLGAIVSCY | and BMP-1 site) |
| | GKTKCTASNKNRGIIKTFSNGCDYVSNKGVDTVSVGNTLYYVNKQEGKSLYVKGEP | without signal |
| | IINFYDPLVFPSDEFDASISQVNEKINQSLAFIRKSDELLHNVNAGKRSANVVRDR | peptide, 741 aa |
| | DLEVDTTLKSLSQQIENIRSPEGSRKNPARTCRDLKMCHSDWKSGEYWIDPNQGCN | (R109A/R136A/ |
| | LDAIKVFCNMETGETCVYPTQPSVAQKNWYISKNPKDKRHVWFGESMTDGFQFEYG | E161P/S215P |
| | GQGSDPADVAIQLTFLRLMSTEASQNITYHCKNSVAYMDQQTGNLKKALLLQGSNE | Mutant) |
| | IEIRAEGNSRFTYSVTVDGCTSHTGAWGKTVIEYKTTKTSRLPIIDVAPLDVGAPD | |
| | QEFGFDVGPVCFL | |
| 14 | MELLILKANAITTILTAVTFCFASGQNITEEFYQSTCSAVSKGYLSALRTGWYTSV | RSV fusion |
| | ITIELSNIKENKCNGTDAKVKLIKQELDKYKNAVTELQLLMQSTPATNNRARAELP | glycoprotein F- |
| | RFMNYTLNNAKKTNVTLSKKRKRAFLGFLLGVGSAIASGVAVSKVLHLPGEVNKIK | Trimer fusion |
| | SALLSTNKAVVSLSNGVSVLTSKVLDLKNYIDKQLLPIVNKQSCSIPNIATVIEFQ | polypeptide F- |
| | QKNNRLLEITREFSVNAGVTTPVSTYMLTNSELLSLINDMPITNDQKKLMSNNVQI | CICP (no |
| | VRQQSYSIMSIIKEEVLAYVVQLPLYGVIDTPCWKLHTSPLCTTNTKEGSNICLTR | glycine repeat |
| | TDRGWYCDNAGSVSFFPQAETCKVQSNRVFCDTMNSLTLPSEVNLCNVDIFNPKYD | and BMP-1 site) |
| | CKIMTSKTDVSSSVITSLGAIVSCYGKTKCTASNKNRGIIKTFSNGCDYVSNKGVD | with signal |
| | TVSVGNTLYYVNKQEGKSLYVKGEPIINFYDPLVFPSDEFDASISQVNEKINQSLA | peptide, 766 aa |
| | FIRKSDELLHNVNAGKRSANVVRDRDLEVDTTLKSLSQQIENIRSPEGSRKNPART | (R109A/R136A/ |
| | CRDLKMCHSDWKSGEYWIDPNQGCNLDAIKVFCNMETGETCVYPTQPSVAQKNWYI | E161P/S215P |
| | SKNPKDKRHVWFGESMTDGFQFEYGGQGSDPADVAIQLTFLRLMSTEASQNITYHC | Mutant) |
| | KNSVAYMDQQTGNLKKALLLQGSNEIEIRAEGNSRFTYSVTVDGCTSHTGAWGKTV | |
| | IEYKTTKTSRLPIIDVAPLDVGAPDQEFGFDVGPVCFL | |
| 15 | QNITEEFYQSTCSAVSKGYLSALRTGWYTSVITIELSNIKENKCNGTDAKVKLIKQ | RSV fusion |
| | ELDKYKNAVTELQLLMQSTPPTNNRARRELPRFMNYTLNNAKKTNVTLSKKRKRRF | glycoprotein F- |
| | LGFLLGVGSAIASGVAVSKVLHLEGEVNKIKSALLSTNKAVVSLSNGVSVLTSKVL | Trimer fusion |
| | DLKNYIDKQLLPIVNKQSCSISNIETVIEFQQKNNRLLEITREFSVNAGVTTPVST | polypeptide |
| | YMLTNSELLSLINDMPITNDQKKLMSNNVQIVRQQSYSIMSIIKEEVLAYVVQLPL | without signal |
| | YGVIDTPCWKLHTSPLCTTNTKEGSNICLTRTDRGWYCDNAGSVSFFPQAETCKVQ | peptide, 806 aa |
| | SNRVFCDTMNSLTLPSEINLCNVDIFNPKYDCKIMTSKTDVSSSVITSLGAIVSCY | (P102, E218, |
| | GKTKCTASNKNRGIIKTFSNGCDYVSNKGMDTVSVGNTLYYVNKQEGKSLYVKGEP | I379, M447) |
| | IINFYDPLVFPSDEFDASISQVNEKINQSLAFIRKSDELLHNVNAGKRSNGLPGPI | |
| | GPPGPRGRTGDAGPVGPPGPPGPPGPPGPPSAGFDFSFLPQPPQEKAHDGGRYYRA | |
| | NDANVVRDRDLEVDTTLKSLSQQIENIRSPEGSRKNPARTCRDLKMCHSDWKSGEY | |
| | WIDPNQGCNLDAIKVFCNMETGETCVYPTQPSVAQKNWYISKNPKDKRHVWFGESM | |
| | TDGFQFEYGGQGSDPADVAIQLTFLRLMSTEASQNITYHCKNSVAYMDQQTGNLKK | |
| | ALLLQGSNEIEIRAEGNSRFTYSVTVDGCTSHTGAWGKIVIEYKTTKTSRLPIIDV | |
| | APLDVGAPDQEFGFDVGPVCFL | |
| 16 | MELLILKANAITTILTAVTFCFASGQNITEEFYQSTCSAVSKGYLSALRTGWYTSV | RSV fusion |
| | ITIELSNIKENKCNGTDAKVKLIKQELDKYKNAVTELQLLMQSTPPTNNRARRELP | glycoprotein F- |
| | RFMNYTLNNAKKTNVTLSKKRKRRFLGFLLGVGSAIASGVAVSKVLHLEGEVNKIK | Trimer fusion |
| | SALLSTNKAVVSLSNGVSVLTSKVLDLKNYIDKQLLPIVNKQSCSISNIETVIEFQ | polypeptide |
| | QKNNRLLEITREFSVNAGVTTPVSTYMLTNSELLSLINDMPITNDQKKLMSNNVQI | with signal |
| | VRQQSYSIMSIIKEEVLAYVVQLPLYGVIDTPCWKLHTSPLCTTNTKEGSNICLTR | peptide, 831 aa |
| | TDRGWYCDNAGSVSFFPQAETCKVQSNRVFCDTMNSLTLPSEINLCNVDIFNPKYD | (P102, E218, |
| | CKIMTSKTDVSSSVITSLGAIVSCYGKTKCTASNKNRGIIKTFSNGCDYVSNKGMD | I379, M447) |
| | TVSVGNTLYYVNKQEGKSLYVKGEPIINFYDPLVFPSDEFDASISQVNEKINQSLA | |
| | FIRKSDELLHNVNAGKRSNGLPGPIGPPGPRGRTGDAGPVGPPGPPGPPGPPGPPS | |
| | AGFDFSFLPQPPQEKAHDGGRYYRANDANVVRDRDLEVDTTLKSLSQQIENIRSPE | |
| | GSRKNPARTCRDLKMCHSDWKSGEYWIDPNQGCNLDAIKVFCNMETGETCVYPTQP | |
| | SVAQKNWYISKNPKDKRHVWFGESMTDGFQFEYGGQGSDPADVAIQLTFLRLMSTE | |
| | ASQNITYHCKNSVAYMDQQTGNLKKALLLQGSNEIEIRAEGNSRFTYSVTVDGCTS | |
| | HTGAWGKTVIEYKTTKTSRLPIIDVAPLDVGAPDQEFGFDVGPVCFL | |
| 17 | QNITEEFYQSTCSAVSKGYLSALRTGWYTSVITIELSNIKENKCNGIDAKVKLIKQ | RSV fusion |
| | ELDKYKNAVTELQLLMQSTPATNNRARRELPRFMNYTLNNAKKTNVTLSKKRKRRF | glycoprotein F |
| | LGFLLGVGSAIASGVAVSKVLHLEGEVNKIKSALLSTNKAVVSLSNGVSVLTSKVL | ectodomain |
| | DLKNYIDKQLLPIVNKQSCSISNIATVIEFQQKNNRLLETTREFSVNAGVTTPVST | without signal |
| | YMLTNSELLSLINDMPITNDQKKLMSNNVQIVRQQSYSIMSIIKEEVLAYVVQLPL | peptide |
| | YGVIDTPCWKLHTSPLCTTNTKEGSNICLTRTDRGWYCDNAGSVSFFPQAETCKVQ | |
| | SNRVFCDTMNSLTLPSEVNLCNVDIFNPKYDCKIMTSKTDVSSSVITSLGAIVSCY | |
| | GKTKCTASNKNRGIIKTFSNGCDYVSNKGVDTVSVGNTLYYVNKQEGKSLYVKGEP | |
| | IINFYDPLVFPSDEFDASISQVNEKINQSLAFIRKSDELLHNVNAGK | |
| 18 | MELLILKANAITTILTAVTFCFASGQNITEEFYQSTCSAVSKGYLSALRTGWYTSV | RSV fusion |
| | ITIELSNIKENKCNGTDAKVKLIKQELDKYKNAVTELQLLMQSTPATNNRARRELP | glycoprotein F |
| | RFMNYTLNNAKKTNVTLSKKRKRRFLGFLLGVGSAIASGVAVSKVLHLEGEVNKIK | ectodomain with |
| | SALLSTNKAVVSLSNGVSVLTSKVLDLKNYIDKQLLPIVNKQSCSISNIATVIEFQ | signal peptide |
| | QKNNRLLEITREFSVNAGVTTPVSTYMLTNSELLSLINDMPITNDQKKLMSNNVQI | |
| | VRQQSYSIMSIIKEEVLAYVVQLPLYGVIDTPCWKLHTSPLCTTNTKEGSNICLTR | |
| | TDRGWYCDNAGSVSFFPQAETCKVQSNRVFCDTMNSLTLPSEVNLCNVDIFNPKYD | |
| | CKIMTSKTDVSSSVITSLGAIVSCYGKTKCTASNKNRGIIKTFSNGCDYVSNKGVD | |
| | TVSVGNTLYYVNKQEGKSLYVKGEPIINFYDPLVFPSDEFDASISQVNEKINQSLA | |
| | FIRKSDELLHNVNAGK | |
| 19 | QNITEEFYQSTCSAVSKGYLSALRTGWYTSVITIELSNIKENKCNGTDAKVKLIKQ | RSV fusion |
| | ELDKYKNAVTELQLLMQSTPATNNRARAELPREMNYTLNNAKKTNVTLSKKRKRRF | glycoprotein F |
| | LGFLLGVGSAIASGVAVSKVLHLEGEVNKIKSALLSTNKAVVSLSNGVSVLTSKVL | ectodomain |
| | DLKNYIDKQLLPIVNKQSCSISNIATVIEFQQKNNRLLEITREFSVNAGVTTPVST | without signal |
| | YMLTNSELLSLINDMPITNDQKKLMSNNVQIVRQQSYSIMSIIKEEVLAYVVQLPL | peptide (R109A |
| | YGVIDTPCWKLHTSPLCTTNTKEGSNICLTRTDRGWYCDNAGSVSFFPQAETCKVQ | Mutant) |
| | SNRVFCDTMNSLTLPSEVNLCNVDIFNPKYDCKIMTSKTDVSSSVITSLGAIVSCY | |
| | GKTKCTASNKNRGIIKTFSNGCDYVSNKGVDTVSVGNTLYYVNKQEGKSLYVKGEP | |
| | IINFYDPLVFPSDEFDASISQVNEKINQSLAFIRKSDELLHNVNAGK | |
| 20 | MELLILKANAITTILTAVTFCFASGQNITEEFYQSTCSAVSKGYLSALRTGWYTSV | RSV fusion |
| | ITIELSNIKENKCNGTDAKVKLIKQELDKYKNAVTELQLLMQSTPATNNRARAELP | glycoprotein F |
| | RFMNYTLNNAKKTNVTLSKKRKRRFLGFLLGVGSAIASGVAVSKVLHLEGEVNKIK | ectodomain with |
| | SALLSTNKAVVSLSNGVSVLTSKVLDLKNYIDKQLLPIVNKQSCSISNIATVIEFQ | signal peptide |
| | QKNNRLLEITREFSVNAGVTTPVSTYMLTNSELLSLINDMPITNDQKKLMSNNVQI | (R109A Mutant) |
| | VRQQSYSIMSIIKEEVLAYVVQLPLYGVIDTPCWKLHTSPLCTTNTKEGSNICLTR | |
| | TDRGWYCDNAGSVSFFPQAETCKVQSNRVFCDTMNSLTLPSEVNLCNVDIFNPKYD | |
| | CKIMTSKTDVSSSVITSLGAIVSCYGKTKCTASNKNRGIIKTFSNGCDYVSNKGVD | |
| | TVSVGNTLYYVNKQEGKSLYVKGEPIINFYDPLVFPSDEFDASISQVNEKINQSLA | |
| | FIRKSDELLHNVNAGK | |
| 21 | QNITEEFYQSTCSAVSKGYLSALRTGWYTSVITIELSNIKENKCNGTDAKVKLIKQ | RSV fusion |
| | ELDKYKNAVTELQLLMQSTPATNNRARRELPRFMNYTLNNAKKTNVTLSKKRKRAF | glycoprotein F |
| | LGFLLGVGSAIASGVAVSKVLHLEGEVNKIKSALLSTNKAVVSLSNGVSVLTSKVL | ectodomain |
| | DLKNYIDKQLLPIVNKQSCSISNIATVIEFQQKNNRLLEITREFSVNAGVTTPVST | without signal |
| | YMLTNSELLSLINDMPITNDQKKLMSNNVQIVRQQSYSIMSIIKEEVLAYVVQLPL | peptide (R136A |
| | YGVIDTPCWKLHTSPLCTTNTKEGSNICLTRTDRGWYCDNAGSVSFFPQAETCKVQ | Mutant) |
| | SNRVFCDTMNSLTLPSEVNLCNVDIFNPKYDCKIMTSKTDVSSSVITSLGAIVSCY | |
| | GKTKCTASNKNRGIIKTFSNGCDYVSNKGVDTVSVGNTLYYVNKQEGKSLYVKGEP | |
| | IINFYDPLVFPSDEFDASISQVNEKINQSLAFIRKSDELLHNVNAGKRSNGLPGPI | |
| | GPPGPRGRTGDAGPVGPPGPPGPPGPPGPPSAGFDFSFLPQPPQEKAHDGGRYYRA | |
| | NDANVVRDRDLEVDTTLKSLSQQIENIRSPEGSRKNPARTCRDLKMCHSDWKSGEY | |
| | WIDPNQGCNLDAIKVFCNMETGETCVYPTQPSVAQKNWYISKNPKDKRHVWEGESM | |
| | TDGFQFEYGGQGSDPADVAIQLTFLRLMSTEASQNITYHCKNSVAYMDQQTGNLKK | |
| | ALLLQGSNEIEIRAEGNSRFTYSVTVDGCTSHTGAWGKIVIEYKTTKTSRLPIIDV | |
| | APLDVGAPDQEFGFDVGPVCFL | |
| 22 | MELLILKANAITTILTAVTFCFASGQNITEEFYQSTCSAVSKGYLSALRTGWYTSV | RSV fusion |
| | ITIELSNIKENKCNGTDAKVKLIKQELDKYKNAVTELQLLMQSTPATNNRARRELP | glycoprotein F |
| | RFMNYTLNNAKKTNVTLSKKRKRAFLGFLLGVGSAIASGVAVSKVLHLEGEVNKIK | ectodomain with |
| | SALLSTNKAVVSLSNGVSVLTSKVLDLKNYIDKQLLPIVNKQSCSISNIATVIEFQ | signal peptide |
| | QKNNRLLEITREFSVNAGVTTPVSTYMLTNSELLSLINDMPITNDQKKLMSNNVQI | (R136A Mutant) |
| | VRQQSYSIMSIIKEEVLAYVVQLPLYGVIDTPCWKLHTSPLCTTNTKEGSNICLTR | |
| | TDRGWYCDNAGSVSFFPQAETCKVQSNRVFCDTMNSLTLPSEVNLCNVDIFNPKYD | |
| | CKIMTSKTDVSSSVITSLGAIVSCYGKTKCTASNKNRGIIKTFSNGCDYVSNKGVD | |
| | TVSVGNTLYYVNKQEGKSLYVKGEPIINFYDPLVFPSDEFDASISQVNEKINQSLA | |
| | FIRKSDELLHNVNAGK | |
| 23 | QNITEEFYQSTCSAVSKGYLSALRTGWYTSVITIELSNIKENKCNGTDAKVKLIKQ | RSV fusion |
| | ELDKYKNAVTELQLLMQSTPATNNRARAELPREMNYTLNNAKKTNVTLSKKRKRAF | glycoprotein F |
| | LGFLLGVGSAIASGVAVSKVLHLEGEVNKIKSALLSTNKAVVSLSNGVSVLTSKVL | ectodomain |
| | DLKNYIDKQLLPIVNKQSCSISNIATVIEFQQKNNRLLEITREFSVNAGVTTPVST | without signal |
| | YMLTNSELLSLINDMPITNDQKKLMSNNVQIVRQQSYSIMSIIKEEVLAYVVQLPL | peptide |
| | YGVIDTPCWKLHTSPLCTTNTKEGSNICLTRTDRGWYCDNAGSVSFFPQAETCKVQ | (R109A/R136A |
| | SNRVFCDTMNSLTLPSEVNLCNVDIFNPKYDCKIMTSKTDVSSSVITSLGAIVSCY | Mutant) |
| | GKTKCTASNKNRGIIKTFSNGCDYVSNKGVDTVSVGNTLYYVNKQEGKSLYVKGEP | |
| | IINFYDPLVFPSDEFDASISQVNEKINQSLAFIRKSDELLHNVNAGK | |
| 24 | MELLILKANAITTILTAVTFCFASGQNITEEFYQSTCSAVSKGYLSALRTGWYTSV | RSV fusion |
| | ITIELSNIKENKCNGTDAKVKLIKQELDKYKNAVTELQLLMQSTPATNNRARAELP | glycoprotein F |
| | RFMNYTLNNAKKTNVTLSKKRKRAFLGFLLGVGSAIASGVAVSKVLHLEGEVNKIK | ectodomain with |
| | SALLSTNKAVVSLSNGVSVLTSKVLDLKNYIDKQLLPIVNKQSCSISNIATVIEFQ | signal peptide |
| | QKNNRLLEITREFSVNAGVTTPVSTYMLTNSELLSLINDMPITNDQKKLMSNNVQI | (R109A/R136A |
| | VRQQSYSIMSIIKEEVLAYVVQLPLYGVIDTPCWKLHTSPLCTTNTKEGSNICLTR | Mutant) |
| | TDRGWYCDNAGSVSFFPQAETCKVQSNRVFCDTMNSLTLPSEVNLCNVDIFNPKYD | |
| | CKIMTSKTDVSSSVITSLGAIVSCYGKTKCTASNKNRGIIKTFSNGCDYVSNKGVD | |
| | TVSVGNTLYYVNKQEGKSLYVKGEPIINFYDPLVFPSDEFDASISQVNEKINQSLA | |
| | FIRKSDELLHNVNAGK | |
| 25 | QNITEEFYQSTCSAVSKGYLSALRTGWYTSVITIELSNIKENKCNGTDAKVKLIKQ | RSV fusion |
| | ELDKYKNAVTELQLLMQSTPATNNRARRELPREMNYTLNNAKKTNVTLSKKRKRRF | glycoprotein F |
| | LGFLLGVGSAIASGVAVSKVLHLPGEVNKIKSALLSTNKAVVSLSNGVSVLTSKVL | ectodomain |
| | DLKNYIDKQLLPIVNKQSCSIPNIATVIEFQQKNNRLLEITREFSVNAGVTTPVST | without signal |
| | YMLTNSELLSLINDMPITNDQKKLMSNNVQIVRQQSYSIMSIIKEEVLAYVVQLPL | peptide |
| | YGVIDTPCWKLHTSPLCTTNTKEGSNICLTRTDRGWYCDNAGSVSFFPQAETCKVQ | (E161P/S215P |
| | SNRVFCDTMNSLTLPSEVNLCNVDIFNPKYDCKIMTSKTDVSSSVITSLGAIVSCY | Mutant) |
| | GKTKCTASNKNRGIIKTFSNGCDYVSNKGVDTVSVGNTLYYVNKQEGKSLYVKGEP | |
| | IINFYDPLVFPSDEFDASISQVNEKINQSLAFIRKSDELLHNVNAGK | |
| 26 | MELLILKANAITTILTAVTFCFASGQNITEEFYQSTCSAVSKGYLSALRTGWYTSV | RSV fusion |
| | ITIELSNIKENKCNGTDAKVKLIKQELDKYKNAVTELQLLMQSTPATNNRARRELP | glycoprotein F |
| | RFMNYTLNNAKKTNVTLSKKRKRRFLGFLLGVGSAIASGVAVSKVLHLPGEVNKIK | ectodomain with |
| | SALLSTNKAVVSLSNGVSVLTSKVLDLKNYIDKQLLPIVNKQSCSIPNIATVIEFQ | signal peptide |
| | QKNNRLLEITREFSVNAGVTTPVSTYMLTNSELLSLINDMPITNDQKKLMSNNVQI | (E161P/S215P |
| | VRQQSYSIMSIIKEEVLAYVVQLPLYGVIDTPCWKLHTSPLCTTNTKEGSNICLTR | Mutant) |
| | TDRGWYCDNAGSVSFFPQAETCKVQSNRVFCDTMNSLTLPSEVNLCNVDIFNPKYD | |
| | CKIMTSKTDVSSSVITSLGAIVSCYGKTKCTASNKNRGIIKTFSNGCDYVSNKGVD | |
| | TVSVGNTLYYVNKQEGKSLYVKGEPIINFYDPLVFPSDEFDASISQVNEKINQSLA | |
| | FIRKSDELLHNVNAGK | |
| 27 | QNITEEFYQSTCSAVSKGYLSALRTGWYTSVITIELSNIKENKCNGTDAKVKLIKQ | RSV fusion |
| | ELDKYKNAVTELQLLMQSTPATNNRARAELPREMNYTLNNAKKTNVTLSKKRKRAF | glycoprotein F |
| | LGFLLGVGSAIASGVAVSKVLHLPGEVNKIKSALLSTNKAVVSLSNGVSVLTSKVL | ectodomain |
| | DLKNYIDKQLLPIVNKQSCSIPNIATVIEFQQKNNRLLEITREFSVNAGVTTPVST | without signal |
| | YMLTNSELLSLINDMPITNDQKKLMSNNVQIVRQQSYSIMSIIKEEVLAYVVQLPL | peptide |
| | YGVIDTPCWKLHTSPLCTTNTKEGSNICLTRTDRGWYCDNAGSVSFFPQAETCKVQ | (R109A/R136A/ |
| | SNRVFCDTMNSLTLPSEVNLCNVDIFNPKYDCKIMTSKTDVSSSVITSLGAIVSCY | E161P/S215P |
| | GKTKCTASNKNRGIIKTFSNGCDYVSNKGVDTVSVGNTLYYVNKQEGKSLYVKGEP | Mutant) |
| | IINFYDPLVFPSDEFDASISQVNEKINQSLAFIRKSDELLHNVNAGK | |
| 28 | MELLILKANAITTILTAVTFCFASGQNITEEFYQSTCSAVSKGYLSALRTGWYTSV | RSV fusion |
| | ITIELSNIKENKCNGTDAKVKLIKQELDKYKNAVTELQLLMQSTPATNNRARAELP | glycoprotein F |
| | RFMNYTLNNAKKTNVTLSKKRKRAFLGFLLGVGSAIASGVAVSKVLHLPGEVNKIK | ectodomain with |
| | SALLSTNKAVVSLSNGVSVLTSKVLDLKNYIDKQLLPIVNKQSCSIPNIATVIEFQ | signal peptide |
| | QKNNRLLEITREFSVNAGVTTPVSTYMLTNSELLSLINDMPITNDQKKLMSNNVQI | (R109A/R136A/ |
| | VRQQSYSIMSIIKEEVLAYVVQLPLYGVIDTPCWKLHTSPLCTTNTKEGSNICLTR | E161P/S215P |
| | TDRGWYCDNAGSVSFFPQAETCKVQSNRVFCDTMNSLTLPSEVNLCNVDIFNPKYD | Mutant) |
| | CKIMTSKTDVSSSVITSLGAIVSCYGKTKCTASNKNRGIIKTFSNGCDYVSNKGVD | |
| | TVSVGNTLYYVNKQEGKSLYVKGEPIINFYDPLVFPSDEFDASISQVNEKINQSLA | |
| | FIRKSDELLHNVNAGK | |
| 29 | QNITEEFYQSTCSAVSKGYLSALRTGWYTSVITIELSNIKENKCNGTDAKVKLIKQ | RSV fusion |
| | ELDKYKNAVTELQLLMQSTPPTNNRARRELPREMNYTLNNAKKTNVTLSKKRKRRF | glycoprotein F |
| | LGFLLGVGSAIASGVAVSKVLHLEGEVNKIKSALLSTNKAVVSLSNGVSVLTSKVL | ectodomain |
| | DLKNYIDKQLLPIVNKQSCSISNIETVIEFQQKNNELLEITREFSVNAGVTTPVST | without signal |
| | YMLTNSELLSLINDMPITNDQKKLMSNNVQIVRQQSYSIMSIIKEEVLAYVVQLPL | peptide (P102, |
| | YGVIDTPCWKLHISPLCTTNTKEGSNICLTRTDRGWYCDNAGSVSFFPQAETCKVQ | E218, I379, |
| | SNRVFCDTMNSLTLPSEINLCNVDIFNPKYDCKIMTSKTDVSSSVITSLGAIVSCY | M447) |
| | GKTKCTASNKNRGIIKTFSNGCDYVSNKGMDTVSVGNTLYYVNKQEGKSLYVKGEP | |
| | IINFYDPLVFPSDEFDASISQVNEKINQSLAFIRKSDELLHNVNAGK | |
| 30 | MELLILKANAITTILTAVTFCFASGQNITEEFYQSTCSAVSKGYLSALRTGWYTSV | RSV fusion |
| | ITIELSNIKENKCNGTDAKVKLIKQELDKYKNAVTELQLLMQSTPPINNRARRELP | glycoprotein F |
| | RFMNYTLNNAKKTNVTLSKKRKRRFLGFLLGVGSAIASGVAVSKVLHLEGEVNKIK | ectodomain with |
| | SALLSTNKAVVSLSNGVSVLTSKVLDLKNYIDKQLLPIVNKQSCSISNIETVIEFQ | signal peptide |
| | QKNNRLLEITREFSVNAGVTTPVSTYMLTNSELLSLINDMPITNDQKKLMSNNVQI | (P102, E218, |
| | VRQQSYSIMSIIKEEVLAYVVQLPLYGVIDTPCWKLHTSPLCTTNTKEGSNICLTR | I379, M447) |
| | TDRGWYCDNAGSVSFFPQAETCKVQSNRVFCDTMNSLTLPSEINLCNVDIFNPKYD | |
| | CKIMTSKTDVSSSVITSLGAIVSCYGKTKCTASNKNRGIIKTFSNGCDYVSNKGMD | |
| | TVSVGNTLYYVNKQEGKSLYVKGEPIINFYDPLVFPSDEFDASISQVNEKINQSLA | |
| | FIRKSDELLHNVNAGK | |
| 31 | MELLILKANAITTILTAVTFCFASGQNITEEFYQSTCSAVSKGYLSALRTGWYTSV | RSV fusion |
| | ITIELSNIKENKCNGTDAKVKLIKQELDKYKNAVTELQLLMQSTPPTNNRARRELP | glycoprotein |
| | RFMNYTLNNAKKTNVTLSKKRKRRFLGFLLGVGSAIASGVAVSKVLHLEGEVNKIK | F0, 574 aa |
| | SALLSTNKAVVSLSNGVSVLTSKVLDLKNYIDKQLLPIVNKQSCSISNIETVIEFQ | (P102, E218, |
| | QKNNRLLEITREFSVNAGVTTPVSTYMLTNSELLSLINDMPITNDQKKLMSNNVQI | I379, M447) |
| | VRQQSYSIMSIIKEEVLAYVVQLPLYGVIDTPCWKLHTSPLCTTNTKEGSNICLTR | |
| | TDRGWYCDNAGSVSFFPQAETCKVQSNRVFCDTMNSLTLPSEINLCNVDIFNPKYD | |
| | CKIMTSKTDVSSSVITSLGAIVSCYGKTKCTASNKNRGIIKTFSNGCDYVSNKGMD | |
| | TVSVGNTLYYVNKQEGKSLYVKGEPIINFYDPLVFPSDEFDASISQVNEKINQSLA | |
| | FIRKSDELLHNVNAGKSTTNIMITTIIIVIIVILLSLIAVGLLLYCKARSTPVTLS | |
| | KDQLSGINNIAFSN | |
| 32 | MELLILKANAITTILTAVTFCFASGQNITEEFYQSTCSAVSKGYLSALRTGWYTSV | RSV fusion |
| | ITIELSNIKENKCNGTDAKVKLIKQELDKYKNAVTELQLLMQSTPATNNRARRELP | glycoprotein |
| | RFMNYTLNNAKKTNVTLSKKRKRRFLGFLLGVGSAIASGVAVSKVLHLEGEVNKIK | F0, 574 aa |
| | SALLSTNKAVVSLSNGVSVLTSKVLDLKNYIDKQLLPIVNKQSCSISNIATVIEFQ | (A102, A218, |
| | QKNNRLLEITREFSVNAGVTTPVSTYMLTNSELLSLINDMPITNDQKKLMSNNVQI | V379, V447) |
| | VRQQSYSIMSIIKEEVLAYVVQLPLYGVIDTPCWKLHTSPLCTTNTKEGSNICLTR | |
| | TDRGWYCDNAGSVSFFPQAETCKVQSNRVFCDTMNSLTLPSEVNLCNVDIFNPKYD | |
| | CKIMTSKTDVSSSVITSLGAIVSCYGKTKCTASNKNRGIIKTFSNGCDYVSNKGVD | |
| | TVSVGNTLYYVNKQEGKSLYVKGEPIINFYDPLVFPSDEFDASISQVNEKINQSLA | |
| | FIRKSDELLHNVNAGKSTINIMITTIIIVIIVILLSLIAVGLLLYCKARSTPVTLS | |
| | KDQLSGINNIAFSN | |
| 33 | MELLILKANAITTILTAVTFCFASG | RSV fusion |
| | | glycoprotein F |
| | | signal peptide, |
| | | 25 aa (1-25 of |
| | | SEQ ID NO: 31 |
| | | or 32) |
| 34 | QNITEEFYQSTCSAVSKGYLSALRTGWYTSVITIELSNIKENKCNGTDAKVKLIKQ | RSV fusion |
| | ELDKYKNAVTELQLLMQSTPPTNNRARR | glycoprotein F2 |
| | | (26-109 of SEQ |
| | | ID NO: 31) |
| 35 | QNITEEFYQSTCSAVSKGYLSALRTGWYTSVITIELSNIKENKCNGTDAKVKLIKQ | RSV fusion |
| | ELDKYKNAVTELQLLMQSTPATNNRARR | glycoprotein F2 |
| | | (26-109 of SEQ |
| | | ID NO: 32) |
| 36 | RARR | Furin site I |
| | | (106-109 of SEQ |
| | | ID NO: 31 or |
| | | 32) |
| 37 | RARA | Furin site I |
| | | mutant (R109A) |
| 38 | ELPREMNYTLNNAKKTNVTLSKKRKRR | RSV fusion |
| | | glycoprotein F |
| | | pep27 (110-136 |
| | | of SEQ ID NO: |
| | | 31 or 32) |
| 39 | KKRKRR | Furin site II |
| | | (131-136 of SEQ |
| | | ID NO: 31 or 32) |
| 40 | KKRKRA | Furin site II |
| | | mutant (R136A) |
| 41 | FLGFLLGVGSAIASGVAVS | RSV fusion |
| | | glycoprotein F |
| | | FP (137-155 of |
| | | SEQ ID NO: 31 |
| | | or 32) |
| 42 | FLGFLLGVGSAIASGVAVSKVLHLEGEVNKIKSALLSTNKAVVSLSNGVSVLTSKV | RSV fusion |
| | LDLKNYIDKQLLPIVNKQSCSISNIETVIEFQQKNNRLLEITREFSVNAGVTTPVS | glycoprotein Fl |
| | TYMLTNSELLSLINDMPITNDQKKLMSNNVQIVRQQSYSIMSIIKEEVLAYVVQLP | (137-524 of SEQ |
| | LYGVIDTPCWKLHTSPLCTTNTKEGSNICLTRTDRGWYCDNAGSVSFFPQAETCKV | ID NO: 31) |
| | QSNRVFCDTMNSLTLPSEINLCNVDIFNPKYDCKIMTSKTDVSSSVITSLGAIVSC | |
| | YGKTKCTASNKNRGIIKTFSNGCDYVSNKGMDTVSVGNTLYYVNKQEGKSLYVKGE | |
| | PIINFYDPLVFPSDEFDASISQVNEKINQSLAFIRKSDELLHNVNAGKSTTN | |
| 43 | FLGFLLGVGSAIASGVAVSKVLHLEGEVNKIKSALLSTNKAVVSLSNGVSVLTSKV | RSV fusion |
| | LDLKNYIDKQLLPIVNKQSCSISNIATVIEFQQKNNRLLEITREFSVNAGVTTPVS | glycoprotein F1 |
| | TYMLTNSELLSLINDMPITNDQKKLMSNNVQIVRQQSYSIMSIIKEEVLAYVVQLP | (137-524 of SEQ |
| | LYGVIDTPCWKLHTSPLCTTNTKEGSNICLTRTDRGWYCDNAGSVSFFPQAEICKV | ID NO: 32) |
| | QSNRVFCDTMNSLTLPSEVNLCNVDIFNPKYDCKIMTSKTDVSSSVITSLGAIVSC | |
| | YGKTKCTASNKNRGIIKTFSNGCDYVSNKGVDTVSVGNTLYYVNKQEGKSLYVKGE | |
| | PIINFYDPLVFPSDEFDASISQVNEKINQSLAFIRKSDELLHNVNAGKSTIN | |
| 44 | IMITTIIIVIIVILLSLIAVGLLLYC | RSV fusion |
| | | glycoprotein F |
| | | transmembrane |
| | | domain (525-550 |
| | | of SEQ ID NO: |
| | | 31 or 32) |
| 45 | KARSTPVTLSKDQLSGINNIAFSN | RSV fusion |
| | | glycoprotein F |
| | | cytoplasmic |
| | | domain (551-574 |
| | | of SEQ ID NO: |
| | | 31 or 32) |
| 46 | KVLHLEGEVNKIKSALLSTNKAVVSLSNGVSVLTSKVLDLKNYIDKQLLPIVNKQS | RSV fusion |
| | CSISNIETVIEFQQKNNRLLEITREFSVNAGVTTPVSTYMLTNSELLSLINDMPIT | glycoprotein F1 |
| | NDQKKLMSNNVQIVRQQSYSIMSIIKEEVLAYVVQLPLYGVIDTPCWKLHTSPLCT | fragment |
| | TNTKEGSNICLTRTDRGWYCDNAGSVSFFPQAETCKVQSNRVFCDIMNSLTLPSEI | (156-520 of SEQ |
| | NLCNVDIFNPKYDCKIMTSKTDVSSSVITSLGAIVSCYGKTKCTASNKNRGIIKTF | ID NO: 31) |
| | SNGCDYVSNKGMDTVSVGNTLYYVNKQEGKSLYVKGEPIINFYDPLVFPSDEFDAS | |
| | ISQVNEKINQSLAFIRKSDELLHNVNAGK | |
| 47 | KVLHLEGEVNKIKSALLSTNKAVVSLSNGVSVLTSKVLDLKNYIDKQLLPIVNKQS | RSV fusion |
| | CSISNIATVIEFQQKNNRLLEITREFSVNAGVTTPVSTYMLTNSELLSLINDMPIT | glycoprotein F1 |
| | NDQKKLMSNNVQIVRQQSYSIMSIIKEEVLAYVVQLPLYGVIDTPCWKLHTSPLCT | fragment |
| | TNTKEGSNICLTRTDRGWYCDNAGSVSFFPQAETCKVQSNRVFCDTMNSLTLPSEV | (156-520 of SEQ |
| | NLCNVDIFNPKYDCKIMTSKTDVSSSVITSLGAIVSCYGKTKCTASNKNRGIIKTF | ID NO: 32) |
| | SNGCDYVSNKGVDTVSVGNTLYYVNKQEGKSLYVKGEPIINFYDPLVFPSDEFDAS | |
| | ISQVNEKINQSLAFIRKSDELLHNVNAGK | |
| 48 | ANVVRDRDLEVDTTLKSLSQQIENIRSPEGSRKNPARTCRDLKMCHSDWKSGEYWI | Trimerization |
| | DPNQGCNLDAIKVFCNMETGETCVYPTQPSVAQKNWYISKNPKDKRHVWFGESMTD | peptide (Type |
| | GFQFEYGGQGSDPADVAIQLTFLRLMSTEASQNITYHCKNSVAYMDQQTGNLKKAL | I), QT version |
| | LLQGSNEIEIRAEGNSRFTYSVIVDGCTSHTGAWGKTVIEYKTTKTSRLPIIDVAP | |
| | LDVGAPDQEFGFDVGPVCFL | |
| 49 | RSANVVRDRDLEVDTTLKSLSQQIENIRSPEGSRKNPARTCRDLKMCHSDWKSGEY | Trimerization |
| | WIDPNQGCNLDAIKVFCNMETGETCVYPTQPSVAQKNWYISKNPKDKRHVWEGESM | peptide (Type |
| | TDGFQFEYGGQGSDPADVAIQLTFLRLMSTEASQNITYHCKNSVAYMDQQTGNLKK | I), QT version |
| | ALLLQGSNEIEIRAEGNSRFTYSVTVDGCTSHTGAWGKTVIEYKTTKTSRLPIIDV | |
| | APLDVGAPDQEFGFDVGPVCFL | |
| 50 | NGLPGPIGPPGPRGRTGDAGPVGPPGPPGPPGPPGPPSAGFDFSFLPQPPQEKAHD | Trimerization |
| | GGRYYRANDANVVRDRDLEVDTTLKSLSQQIENIRSPEGSRKNPARTCRDLKMCHS | peptide (Type |
| | DWKSGEYWIDPNQGCNLDAIKVFCNMETGETCVYPTQPSVAQKNWYISKNPKDKRH | I), with |
| | VWFGESMIDGFQFEYGGQGSDPADVAIQLTFLRLMSTEASQNITYHCKNSVAYMDQ | glycine-X-Y |
| | QTGNLKKALLLQGSNEIEIRAEGNSRFTYSVTVDGCTSHTGAWGKTVIEYKTTKTS | repeats and D→N |
| | RLPIIDVAPLDVGAPDQEFGFDVGPVCFL | mutation at |
| | | BMP-1 site, QT |
| | | version |
| 51 | NGLPGPIGPPGPRGRTGDAGPVGPPGPPGPPGPPGPPSAGFDFSFLPQPPQEKAHD | Trimerization |
| | GGRYYRNDDANVVRDRDLEVDTTLKSLSQQIENIRSPEGSRKNPARTCRDLKMCHS | peptide (Type |
| | DWKSGEYWIDPNQGCNLDAIKVFCNMETGETCVYPTQPSVAQKNWYISKNPKDKRH | I), with |
| | VWFGESMTDGFQFEYGGQGSDPADVAIQLTFLRLMSTEASQNITYHCKNSVAYMDQ | glycine-X-Y |
| | QTGNLKKALLLQGSNEIEIRAEGNSRFTYSVTVDGCTSHTGAWGKTVIEYKTIKTS | repeats and A→N |
| | RLPIIDVAPLDVGAPDQEFGFDVGPVCFL | mutation at |
| | | BMP-1 site, QT |
| | | version |
| 52 | RSNGLPGPIGPPGPRGRTGDAGPVGPPGPPGPPGPPGPPSAGFDFSFLPQPPQEKA | Trimerization |
| | HDGGRYYRANDANVVRDRDLEVDITLKSLSQQIENIRSPEGSRKNPARTCRDIKMC | peptide (Type |
| | HSDWKSGEYWIDPNQGCNLDAIKVFCNMETGETCVYPTQPSVAQKNWYISKNPKDK | I), with |
| | RHVWEGESMTDGFQFEYGGQGSDPADVAIQLTFLRLMSTEASQNITYHCKNSVAYM | glycine-X-Y |
| | DQQTGNLKKALLLQGSNEIEIRAEGNSRFTYSVIVDGCTSHTGAWGKTVIEYKTTK | repeats and D→N |
| | TSRLPIIDVAPLDVGAPDQEFGFDVGPVCFL | mutation at |
| | | BMP-1 site, QT |
| | | version |
| 53 | GSNGLPGPIGPPGPRGRTGDAGPVGPPGPPGPPGPPGPPSAGFDFSFLPQPPQEKA | Trimerization |
| | HDGGRYYRANDANVVRDRDLEVDTTLKSLSQQIENIRSPEGSRKNPARTCRDLKMC | peptide (Type |
| | HSDWKSGEYWIDPNQGCNLDAIKVFCNMETGETCVYPTQPSVAQKNWYISKNPKDK | I), with |
| | RHVWEGESMTDGFQFEYGGQGSDPADVAIQLIFLRLMSTEASQNITYHCKNSVAYM | glycine-X-Y |
| | DQQTGNLKKALLLQGSNEIEIRAEGNSRFTYSVTVDGCTSHTGAWGKTVIEYKTTK | repeats and D→N |
| | TSRLPIIDVAPLDVGAPDQEFGFDVGPVCFL | mutation at |
| | | BMP-1 site, QT |
| | | version |
| 54 | ANVVRDRDLEVDTTLKSLSQQIENIRSPEGSRKNPARTCRDLKMCHSDWKSGEYWI | Trimerization |
| | DPNQGCNLDAIKVFCNMETGETCVYPTQPSVAQKNWYISKNPKDKRHVWFGESMTD | peptide (Type |
| | GFQFEYGGQGSDPADVAIQLTFLRLMSTEASQNITYHCKNSVAYMDQQTGNLKKAL | I), KS version |
| | LLKGSNEIEIRAEGNSRFTYSVTVDGCTSHTGAWGKTVIEYKITKSSRLPIIDVAP | |
| | LDVGAPDQEFGFDVGPVCFL | |
| 55 | RSANVVRDRDLEVDTTLKSLSQQIENIRSPEGSRKNPARTCRDLKMCHSDWKSGEY | Trimerization |
| | WIDPNQGCNLDAIKVFCNMETGETCVYPTQPSVAQKNWYISKNPKDKRHVWEGESM | peptide (Type |
| | TDGFQFEYGGQGSDPADVAIQLTFLRLMSTEASQNITYHCKNSVAYMDQQTGNLKK | I), KS version |
| | ALLLKGSNEIEIRAEGNSRFTYSVIVDGCTSHTGAWGKIVIEYKTTKSSRLPIIDV | |
| | APLDVGAPDQEFGFDVGPVCFL | |
| 56 | NGLPGPIGPPGPRGRTGDAGPVGPPGPPGPPGPPGPPSAGFDFSFLPQPPQEKAHD | Trimerization |
| | GGRYYRANDANVVRDRDLEVDTTLKSLSQQIENIRSPEGSRKNPARTCRDLKMCHS | peptide (Type |
| | DWKSGEYWIDPNQGCNLDAIKVFCNMEIGETCVYPTQPSVAQKNWYISKNPKDKRH | I) with |
| | VWFGESMTDGFQFEYGGQGSDPADVAIQLTFLRLMSTEASQNITYHCKNSVAYMDQ | glycine-X-Y |
| | QTGNLKKALLLKGSNEIEIRAEGNSRFTYSVTVDGCTSHTGAWGKTVIEYKTIKSS | repeats and D→N |
| | RLPIIDVAPLDVGAPDQEFGFDVGPVCFL | mutation at |
| | | BMP-1 site, KS |
| | | version |
| 57 | NGLPGPIGPPGPRGRTGDAGPVGPPGPPGPPGPPGPPSAGFDFSFLPQPPQEKAHD | Trimerization |
| | GGRYYRNDDANVVRDRDLEVDTTLKSLSQQIENIRSPEGSRKNPARTCRDLKMCHS | peptide (Type |
| | DWKSGEYWIDPNQGCNLDAIKVFCNMETGETCVYPTQPSVAQKNWYISKNPKDKRH | I) with |
| | VWEGESMTDGFQFEYGGQGSDPADVAIQLTFLRLMSTEASQNITYHCKNSVAYMDQ | glycine-X-Y |
| | QTGNLKKALLLKGSNEIEIRAEGNSRFTYSVIVDGCTSHTGAWGKTVIEYKTTKSS | repeats and A→N |
| | RLPIIDVAPLDVGAPDQEFGFDVGPVCFL | mutation at |
| | | BMP-1 site, KS |
| | | version |
| 58 | RSNGLPGPIGPPGPRGRTGDAGPVGPPGPPGPPGPPGPPSAGFDFSFLPQPPQEKA | Trimerization |
| | HDGGRYYRANDANVVRDRDLEVDTTLKSLSQQIENIRSPEGSRKNPARTCRDLKMC | peptide (Type |
| | HSDWKSGEYWIDPNQGCNLDAIKVFCNMEIGETCVYPTQPSVAQKNWYISKNPKDK | I) with |
| | RHVWEGESMTDGFQFEYGGQGSDPADVAIQLTFLRLMSTEASQNITYHCKNSVAYM | glycine-X-Y |
| | DQQTGNLKKALLLKGSNEIEIRAEGNSRFTYSVTVDGCISHTGAWGKTVIEYKITK | repeats and D→N |
| | SSRLPIIDVAPLDVGAPDQEFGFDVGPVCFL | mutation at |
| | | BMP-1 site, KS |
| | | version |
| 59 | GSNGLPGPIGPPGPRGRTGDAGPVGPPGPPGPPGPPGPPSAGFDFSFLPQPPQEKA | Trimerization |
| | HDGGRYYRANDANVVRDRDLEVDTTLKSLSQQIENIRSPEGSRKNPARTCRDLKMC | peptide (Type |
| | HSDWKSGEYWIDPNQGCNLDAIKVFCNMETGETCVYPTQPSVAQKNWYISKNPKDK | I) with |
| | RHVWFGESMTDGFQFEYGGQGSDPADVAIQLTFLRLMSTEASQNITYHCKNSVAYM | glycine-X-Y |
| | DQQTGNLKKALLLKGSNEIEIRAEGNSRFTYSVTVDGCTSHTGAWGKTVIEYKTTK | repeats and D→N |
| | SSRLPIIDVAPLDVGAPDQEFGFDVGPVCFL | mutation at |
| | | BMP-1 site, KS |
| | | version |
| 60 | DEIMTSLKSVNGQIESLISPDGSRKNPARNCRDLKFCHPELKSGEYWVDPNQGCKL | Trimerization |
| | DAIKVFCNMETGETCISANPLNVPRKHWWTDSSAEKKHVWFGESMDGGFQFSYGNP | peptide (Type |
| | ELPEDVLDVQLAFLRLLSSRASQNITYHCKNSIAYMDQASGNVKKALKLMGSNEGE | III) |
| | FKAEGNSKFTYTVLEDGCTKHIGEWSKTVFEYRIRKAVRLPIVDIAPYDIGGPDQE | |
| | FGVDVGPVCE | |
| 61 | EPMDFKINTDEIMTSLKSVNGQIESLISPDGSRKNPARNCRDLKFCHPELKSGEYW | Trimerization |
| | VDPNQGCKLDAIKVFCNMETGETCISANPLNVPRKHWWTDSSAEKKHVWFGESMDG | peptide (Type |
| | GFQFSYGNPELPEDVLDVQLAFLRLLSSRASQNITYHCKNSIAYMDQASGNVKKAL | III) |
| | KLMGSNEGEFKAEGNSKFTYTVLEDGCTKHTGEWSKIVFEYRTRKAVRLPIVDIAP | |
| | YDIGGPDQEFGVDVGPVCFL | |
| 62 | SEPMDFKINTDEIMTSLKSVNGQIESLISPDGSRKNPARNCRDLKFCHPELKSGEY | Trimerization |
| | WVDPNQGCKLDAIKVFCNMETGETCISANPLNVPRKHWWTDSSAEKKHVWFGESMD | peptide (Type |
| | GGFQFSYGNPELPEDVLDVQLAFLRLLSSRASQNITYHCKNSIAYMDQASGNVKKA | III) |
| | LKLMGSNEGEFKAEGNSKFTYTVLEDGCTKHTGEWSKTVFEYRTRKAVRLPIVDIA | |
| | PYDIGGPDQEFGVDVGPVCFL | |
| 63 | RSEPMDFKINTDEIMTSLKSVNGQIESLISPDGSRKNPARNCRDLKFCHPELKSGE | Trimerization |
| | YWVDPNQGCKLDAIKVFCNMEIGETCISANPLNVPRKHWWIDSSAEKKHVWFGESM | peptide (Type |
| | DGGFQFSYGNPELPEDVLDVQLAFLRLLSSRASQNITYHCKNSIAYMDQASGNVKK | III) |
| | ALKLMGSNEGEFKAEGNSKFTYTVLEDGCTKHTGEWSKTVFEYRTRKAVRLPIVDI | |
| | APYDIGGPDQEFGVDVGPVCFL | |

1. A method for preventing infection by a respiratory syncytial virus (RSV) in a mammal, comprising immunizing a mammal with an effective amount of a recombinant subunit vaccine comprising a soluble RSV viral surface antigen joined by in-frame fusion to a C-terminal portion of a collagen to form a disulfide bond-linked trimeric fusion protein, wherein the RSV is of subtype A or subtype B.

2. (canceled)

3. The method of claim 1, wherein the RSV viral surface antigen comprises an F protein or a fragment or epitope thereof, and/or an F1 peptide or a fragment or epitope thereof, and/or an F2 peptide and/or a fragment or epitope thereof.

4. (canceled)

5. (canceled)

6. The method of claim 1, wherein the RSV viral surface antigen comprises an F1 peptide or a fragment or epitope thereof and an F2 peptide or a fragment or epitope thereof.

7. The method of claim 1, wherein the RSV viral surface antigen comprises a mutant F protein or a fragment or epitope thereof.

8. The method of claim 1, wherein the RSV viral surface antigen comprises a mutant F1 peptide or a fragment or epitope thereof.

9. The method of claim 1, wherein the RSV viral surface antigen comprises a mutant F2 peptide or a fragment or epitope thereof.

10. The method of any of claim 1, wherein the fusion protein comprises a sequence selected from a sequence set forth in SEQ ID NO: 1, a sequence set forth in SEO ID NO: 3, a sequence set forth in SEO ID NO: 5, a sequence set forth in SEO ID NO: 7, a sequence set forth in SEO ID NO: 9, a sequence set forth in SEO ID NO: 11, a sequence set forth in SEO ID NO: 13, a sequence set forth in SEO ID NO: 15, a sequence set forth in SEO ID NO: 37, a sequence set forth in SEO ID NO: 40, or any combination of the sequences thereof.

11-19. (canceled)

20. The method of claim 1, wherein the fusion protein comprises a first sequence set forth in any of SEQ ID NOs: 17-47 linked to a second sequence set forth in any of SEQ ID NOs: 48-63, wherein the C terminus of the first sequence is directly or indirectly linked to the N terminus of the second sequence.

21. The method of claim 1, wherein the recombinant subunit vaccine is administered via intramuscular injection.

22. The method of claim 1, wherein the recombinant subunit vaccine is administered via intra-nasal spray.

23. The method of claim 1, wherein the recombinant subunit vaccine is administered in a single dose or a series of doses separated by intervals of weeks or months.

24. The method of claim 1, wherein the recombinant subunit vaccine is administered without adjuvant.

25. The method of claim 1, wherein the recombinant subunit vaccine is administered with an adjuvant.

26. The method of claim 1, wherein the recombinant subunit vaccine is administered with more than one adjuvant.

27. A method for detecting antibodies to a respiratory syncytial virus (RSV) from sera of a mammal comprising the step of contacting the sera with a soluble RSV surface antigen joined by in-frame fusion to a collagen to form a disulfide bond-linked trimeric fusion protein.

28. The method of claim 27, wherein the soluble RSV surface antigen is an F protein or peptide.

29. A method of using a recombinant subunit vaccine comprising a soluble surface antigen from a respiratory syncytial virus (RSV), which is joined by in-frame fusion to a collagen to form a disulfide bond-linked trimeric fusion protein, the method comprising: immunizing a mammal, purifying the neutralizing antibody generated, and treating patients infected by the said RSV via passive immunization using said neutralizing antibody.

30. The method of claim 29, wherein the neutralizing antibody comprises polyclonal antibodies.

31. The method of claim 29, wherein the neutralizing antibody is a monoclonal antibody.

32. The method of claim 29, wherein the neutralizing antibody is a monoclonal antibody to an F protein or peptide and/or F1 peptide and/or an F2 peptide.

33-34. (canceled)
